(19) United States
(12) Patent Application Publication
(10) Pub. No.: US 2010/0004292 A1
(43) Pub. Date: Jan. 7, 2010

(54) Iminooxazolidine Derivatives and Their Use

(75) Inventors: Susanne Rohrig, Hilden (DE); Mario Jeske, Solingen (DE); Metin Akbaba, Ratingen (DE); Elisabeth Perzborn, Wuppertal (DE); Christoph Gerdes, Leverkusen (DE); Karl-Heinz Schlemmer, Wuppertal (DE)

Correspondence Address: Barbara A. Shimei;Director, Patents & Licensing, Bayer HealthCare LLC - Pharmaceuticals, 555 White Plains Road, Third Floor, Tarrytown, NY, 10591, US

(73) Assignee: Bayer HealthCare AG, Leverkusen (DE)

(21) Appl. No.: 11/991,670

(22) PCT Filed: Aug. 26, 2006
(86) PCT No.: PCT/EP2006/008390

§ 371 Date: May 5, 2009

(30) Foreign Application Priority Data
Sep. 8, 2005 (DE) 1020005042583.6

(51) Int. Cl. A61K 31/4439; C07D 263/28; C07D 413/14; C07D 277/18; A61K 31/422; A61K 31/427; C12N 5/06; A61P 7/02

(52) U.S. Cl. 514/340; 548/234; 546/271.4; 548/185; 514/377; 514/369; 435/325

(57) ABSTRACT

The present application relates to novel iminooxazolidine derivatives, to processes for their preparation, to their use for the treatment and/or prophylaxis of diseases and also to their use for preparing medicaments for the treatment and/or prophylaxis of diseases, in particular thromboembolic disorders.

The present application relates to novel iminooxazolidine derivatives, to processes for their preparation, to their use for the treatment and/or prophylaxis of diseases and also to their use for preparing medicaments for the treatment and/or prophylaxis of diseases, in particular thromboembolic disorders.

Blood coagulation is a protective mechanism of the organism which helps to “seal” defects in the wall of the blood vessels quickly and reliably. Thus, loss of blood can be avoided or kept to a minimum. Hemostasis after injury of the blood vessels is effected mainly by the coagulation system in which an enzymatic cascade of complex reactions of plasma proteins is triggered. Numerous blood coagulation factors are involved in this process, each of which factors converts, on activation, the respectively next inactive precursor into its active form. At the end of the cascade comes the conversion of soluble fibrinogen into insoluble fibrin, resulting in the formation of a blood clot. In blood coagulation, traditionally the intrinsic and the extrinsic system, which end in a joint reaction path, are distinguished. Here factor Xa, which is formed from the proenzyme factor X, plays a key role, since it connects the two coagulation paths. The activated serine protease Xa cleaves prothrombin to thrombin. The resulting thrombin, in turn, cleaves fibrinogen to fibrin. Subsequent crosslinking of the fibrin monomers causes formation of blood clots and thus hemostasis. In addition, thrombin is a potent effector of platelet aggregation which likewise contributes significantly to hemostasis.

Hemostasis is subject to a complex regulatory mechanism. Uncontrolled activation of the coagulation system or defective inhibition of the activation processes may cause formation of local thrombi or embolisms in vessels (arteries, veins, lymph vessels) or in heart cavities. This may lead to serious thromboembolic disorders. In addition, in the case of consumption coagulopathy, hypercoagulability may—systemically—result in disseminated intravascular coagulation. Thromboembolic complications furthermore occur in microangiopathic hemolytic anaemias, extracorporeal blood circulation, such as hemodialysis, and also in connection with prosthetic heart valves.

Thromboembolic disorders are the most frequent cause of morbidity and mortality in most industrialized countries. [Heart Disease: A Textbook of Cardiovascular Medicine, Eugene Braunwald, 5. edition, 1997, W.B. Saunders Company, Philadelphia].

The anticoagulants, i.e. substances for inhibiting or preventing blood coagulation, which are known from the prior art, have various, often grave disadvantages. Accordingly, in practice, an efficient treatment method or prophylaxis of thromboembolic disorders is very difficult and unsatisfactory.

In the therapy and prophylaxis of thromboembolic disorders, use is firstly made of heparin, which is administered parenterally or subcutaneously. Owing to more favourable pharmacokinetic properties, preference is nowadays more and more given to low-molecular-weight heparin; however, even with low-molecular-weight heparin, it is not possible to avoid the known disadvantages described below, which are involved in heparin therapy. Thus, heparin is ineffective when administered orally and has a relatively short half-life. Since heparin inhibits a plurality of factors of the blood coagulation cascade at the same time, the action is nonselective. Moreover, there is a high risk of bleeding; in particular, brain hemorrhages and gastrointestinal bleeding may occur, which may result in thrombopenia, drug-induced alopecia or osteoporosis [Pschyrembel, Klinisches Wörterbuch, 257th edition, 1994, Walter de Gruyter Verlag, page 610, entry “Heparin”; Römpp Lexikon Chemie, Version 1.5, 1998, Georg Thieme Verlag Stuttgart, entry “Heparin”].

A second class of anticoagulants are the vitamin K antagonists. These include, for example, 1,3-indanediones, and especially compounds such as warfarin, phenprocoumon, dicumarol and other coumarin derivatives which inhibit the synthesis of various products of certain vitamin K-dependent coagulation factors in the liver in a non-selective manner. Owing to the mechanism of action, however, the onset of the action is very slow (latency to the onset of action 36 to 48 hours). It is possible to administer the compounds orally; however, owing to the high risk of bleeding and the narrow therapeutic index, a time-consuming individual adjustment and monitoring of the patient are required [J. Hirsh, J. Dalen, D. R. Anderson et al., “Oral anticoagulants: Mechanism of action, clinical effectiveness, and optimal therapeutic range” Chest 2001, 119, 8S-21S; J. Ansell, J. Hirsh, J. Dalen et al., “Managing oral anticoagulant therapy” Chest 2001, 119, 22S-38S; P. S. Wells, A. M. Holbrook, N. R. Crowther et al., “Interactions of warfarin with drugs and food” Ann. Intern. Med. 1994, 121, 676-683].

Recently, a novel therapeutic approach for the treatment and prophylaxis of thromboembolic disorders has been described. This novel therapeutic approach aims to inhibit factor Xa. Because of the central role which factor Xa plays in the blood coagulation cascade, factor Xa is one of the most important targets for anticoagulants [J. Hauptmann, J. Stürzebecher, Thrombosis Research 1999, 93, 203; S. A. V. Raghavan, M. Dikshit, “Recent advances in the status and targets of antithrombotic agents” Drugs Fut. 2002, 27, 669-683; H. A. Wieland, V. Laux, D. Kozian, M. Lorenz, “Approaches in anticoagulation: Rationales for target positioning” Curr. Opin. Investig. Drugs 2003, 4, 264-271; U. J. Ries, W. Wienen, “Serine proteases as targets for antithrombotic therapy” Drugs Fut. 2003, 28, 355-370; L.-A. Linkins, J. I. Weitz, “New anticoagulant therapy” Annu. Rev. Med. 2005, 56, 63-77 (online publication August 2004)].

It has been shown that, in animal models, various both peptidic and nonpeptidic compounds are effective as factor Xa inhibitors. A large number of direct factor Xa inhibitors is already known [J. M. Walenga, W. P. Jeske, D. Hoppensteadt, J. Fareed, “Factor Xa Inhibitors: Today and beyond” Curr. Opin. Investig. Drugs 2003, 4, 272-281; J. Ruef, H. A. Katus, “New antithrombotic drugs on the horizon” Expert Opin. Investig. Drugs 2003, 12, 781-797; M. L. Quan, J. M. Smallheer, “The race to an orally active Factor Xa inhibitor: Recent advances” Curr. Opin. Drug Discovery & Development 2004, 7, 460-469]. Furthermore, non-peptidic low-molecular weight factor Xa inhibitors are also described, for example, in WO 03/047520, WO 02/079145, WO 02/000651 and WO 02/000647.

It is an object of the present invention to provide novel substances for controlling disorders, in particular thromboembolic disorders.

The present invention provides compounds of the general formula (I)

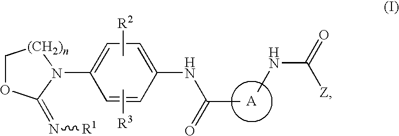

in which
n represents the number 1, 2 or 3,
R¹ represents hydrogen, hydroxyl, (C₁-C₄)-alkyl, (C₁-C₄)-alkanoyl or cyano,
R² and R³ are identical or different and independently of one another represent hydrogen, fluorine, chlorine, cyano, (C₁-C₄)-alkyl, cyclopropyl, trifluoromethyl, hydroxyl, (C₁-C₄)-alkoxy, trifluoromethoxy, amino, mono- or di-(C₁-C₄)-alkylamino,
A represents a phenylene ring or a 5- or 6-membered heteroarylene ring where the two groupings —CO—NH-phenyl and —NH—CO-Z are located at adjacent ring atoms of the phenylene or heteroarylene ring and where phenylene and heteroarylene may additionally be substituted by substituents selected from the group consisting of fluorine, chlorine, cyano, (C₁-C₆)-alkyl, (C₃-C₇)-cycloalkyl, trifluoromethyl, hydroxyl, (C₁-C₆)-alkoxy, trifluoromethoxy, amino, mono- and di-(C₁-C₆)-alkylamino, (C₃-C₇)-cycloalkylamino, (C₁-C₆)-alkanoylamino, (C₁-C₆)-alkoxycarbonylamino, (C₁-C₆)-alkylthio, (C₁-C₆)-alkylsulfonyl, hydroxycarbonyl, (C₁-C₆)-alkoxycarbonyl, aminocarbonyl, mono- and di-(C₁-C₆)-alkylaminocarbonyl,
   where (C₁-C₆)-alkyl, (C₁-C₆)-alkoxy, mono- and di-(C₁-C₆)-alkylamino for their part may each be substituted by hydroxyl, (C₁-C₄)-alkoxy, amino, mono- or di-(C₁-C₄)-alkylamino or (C₃-C₅)-cycloalkylamino,
      and
Z represents phenyl, pyridyl, pyrimidinyl, pyrazinyl or thienyl which may in each case be mono- or disubstituted by identical or different substituents selected from the group consisting of fluorine, chlorine, cyano, methoxy, (C₁-C₄)-alkyl (which for its part may be substituted by amino), ethynyl and amino,
   and salts, solvates and solvates of the salts thereof.

Compounds according to the invention are the compounds of the formula (I) and their salts, solvates and solvates of the salts, the compounds, comprised by formula (I), of the formulae mentioned below and their salts, solvates and solvates of the salts and the compounds, comprised by the formula (I), mentioned below as exemplary embodiments and their salts, solvates and solvates of the salts if the compounds, comprised by formula (I), mentioned below are not already salts, solvates and solvates of the salts.

Depending on their structure, the compounds according to the invention can exist in stereoisomeric forms (enantiomers, diastereomers). Accordingly, the invention comprises the enantiomers or diastereomers and their respective mixtures. From such mixtures of enantiomers and/or diastereomers, it is possible to isolate the stereoisomerically uniform components in a known manner.

If the compounds according to the invention can be present in tautomeric forms, the present invention comprises all tautomeric forms.

In the context of the present invention, preferred salts are physiologically acceptable salts of the compounds according to the invention. The invention also comprises salts which for their part are not suitable for pharmaceutical applications, but which can be used, for example, for isolating or purifying the compounds according to the invention.

Physiologically acceptable salts of the compounds according to the invention include acid addition salts of mineral acids, carboxylic acids and sulfonic acids, for example salts of hydrochloric acid, hydrobromic acid, sulfuric acid, phosphoric acid, methanesulfonic acid, ethanesulfonic acid, toluenesulfonic acid, benzenesulfonic acid, naphthalene disulfonic acid, acetic acid, trifluoroacetic acid, propionic acid, lactic acid, tartaric acid, malic acid, citric acid, fumaric acid, maleic acid and benzoic acid.

Physiologically acceptable salts of the compounds according to the invention also include salts of customary bases, such as, by way of example and by way of preference, alkali metal salts (for example sodium salts and potassium salts), alkaline earth metal salts (for example calcium salts and magnesium salts) and ammonium salts, derived from ammonia or organic amines having 1 to 16 carbon atoms, such as, by way of example and by way of preference, ethylamine, diethylamine, triethylamine, ethyldiisopropylamine, monoethanolamine, diethanolamine, triethanolamine, dicyclohexylamine, dimethylaminoethanol, procaine, dibenzylamine, N-methylmorpholine, arginine, lysine, ethylenediamine and N-methylpiperidine.

In the context of the invention, solvates are those forms of the compounds according to the invention which, in solid or liquid state, form a complex by coordination with solvent molecules. Hydrates are a specific form of the solvates where the coordination is with water. In the context of the present invention, preferred solvates are hydrates.

Moreover, the present invention also comprises prodrugs of the compounds according to the invention. The term “prodrugs” includes compounds which for their part may be biologically active or inactive but which, during the time they spend in the body, are converted into compounds according to the invention (for example metabolically or hydrolytically).

In the context of the present invention, unless specified differently, the substituents have the following meanings:

In the context of the invention, (C₁-C₆)-alkyl and (C₁-C₄)-alkyl represent a straight-chain or branched alkyl radical having 1 to 6 and 1 to 4 carbon atoms, respectively. Preference is given to a straight-chain or branched alkyl radical having 1 to 4 carbon atoms. The following radicals may be mentioned by way of example and by way of preference: methyl, ethyl, n-propyl, isopropyl, n-butyl, isobutyl, sec-butyl, tert-butyl, 1-ethylpropyl, n-pentyl and n-hexyl.

In the context of the invention, (C₃-C₇)-cycloalkyl and (C₃-C₅)-cycloalkyl represent a monocyclic cycloalkyl group having 3 to 7 and 3 to 5 carbon atoms, respectively. Preference is given to a cycloalkyl radical having 3 to 5 carbon atoms. The following radicals may be mentioned by way of example and by way of preference: cyclopropyl, cyclobutyl, cyclopentyl, cyclohexyl and cycloheptyl.

In the context of the invention, (C₁-C₆)-alkoxy and (C₁-C₄)-alkoxy represent a straight-chain or branched alkoxy radical having 1 to 6 and 1 to 4 carbon atoms, respectively. Preference is given to a straight-chain or branched alkoxy radical having 1 to 4 carbon atoms. The following radicals may be mentioned by way of example and by way of preference: methoxy, ethoxy, n-propoxy, isopropoxy, n-butoxy and tert-butoxy.

In the context of the invention, (C₁-C₆)-alkanoyl [(C₁-C₆)-acyl] and (C₁-C₄)-alkanoyl [(C₁-C₄)-acyl] represent a straight-chain or branched alkyl radical having 1 to 6 and 1 to 4 carbon atoms, respectively, which carries a doubly attached oxygen atom in the 1-position and is attached via the 1-position. Preference is given to a straight-chain or branched alkanoyl radical having 1 to 4 carbon atoms. The following radicals may be mentioned by way of example and by way of preference: formyl, acetyl, propionyl, n-butyryl, isobutyryl and pivaloyl.

In the context of the invention, (C₁-C₆)-alkoxycarbonyl represents a straight-chain or branched alkoxy radical having 1 to 6 carbon atoms which is attached via a carbonyl group. Preference is given to a straight-chain or branched alkoxycarbonyl radical having 1 to 4 carbon atoms in the alkoxy group. The following radicals may be mentioned by way of example and by way of preference: methoxycarbonyl, ethoxycarbonyl, n-propoxycarbonyl, isopropoxycarbonyl and tert-butoxycarbonyl.

In the context of the invention, mono-(C₁-C₆)-alkylamino and mono-(C₁-C₄)-alkylamino represent an amino group having a straight-chain or branched alkyl substituent having 1 to 6 and 1 to 4 carbon atoms, respectively. Preference is given to a straight-chain or branched monoalkylamino radical having 1 to 4 carbon atoms. The following radicals may be mentioned by way of example and by way of preference: methylamino, ethylamino, n-propylamino, isopropylamino and tert-butylamino.

In the context of the invention, di-(C₁-C₆)-alkylamino and di-(C₁-C₄)-alkylamino represent an amino group having two identical or different straight-chain or branched alkyl substituents having each 1 to 6 and 1 to 4 carbon atoms, respectively. Preference is given to straight-chain or branched dialkylamino radicals having in each case 1 to 4 carbon atoms. The following radicals may be mentioned by way of example and by way of preference: N,N-dimethylamino, N,N-diethylamino, N-ethyl-N-methylamino, N-methyl-N-n-propylamino, N-isopropyl-N-n-propylamino, N-tert-butyl-N-methylamino, N-ethyl-N-n-pentylamino and N-n-hexyl-N-methylamino.

In the context of the invention, (C₃-C₇)-cycloalkylamino and (C₃-C₅)-cycloalkylamino represent an amino group having a cycloalkyl substituent having 3 to 7 and 3 to 5 carbon atoms, respectively. Preference is given to a cycloalkylamino radical having 3 to 5 carbon atoms. The following radicals may be mentioned by way of example and by way of preference: cyclopropylamino, cyclobutylamino, cyclopentylamino, cyclohexylamino and cycloheptylamino.

In the context of the invention, (C₁-C₆)-alkanoylamino represents an amino group having a straight-chain or branched alkanoyl substituent which has 1 to 6 carbon atoms and is attached via the carbonyl group. Preference is given to an alkanoylamino radical having 1 bis 4 carbon atoms. The following radicals may be mentioned by way of example and by way of preference: form-amido, acetamido, propionamido, n-butyramido and pivaloylamido.

In the context of the invention, (C₁-C₆)-alkoxycarbonylamino represents an amino group having a straight-chain or branched alkoxycarbonyl substituent which has 1 to 6 carbon atoms in the alkoxy radical and is attached via the carbonyl group. Preference is given to an alkoxycarbonylamino radical having 1 bis 4 carbon atoms in the alkoxy group. The following radicals may be mentioned by way of example and by way of preference: methoxycarbonylamino, ethoxycarbonylamino, n-propoxycarbonylamino and tert-butoxycarbonylamino.

In the context of the invention, mono-(C₁-C₆)-alkylaminocarbonyl and mono-(C₁-C₄) alkylaminocarbonyl represent a straight-chain or branched monoalkylamino radical having 1 to 6 and 1 to 4 carbon atoms, respectively, which is attached via a carbonyl group. Preference is given to a straight-chain or branched monoalkylaminocarbonyl radical having 1 to 4 carbon atoms in the alkylamino group. The following radicals may be mentioned by way of example and by way of preference: methylaminocarbonyl, ethylaminocarbonyl, n-propylaminocarbonyl, isopropylamino-carbonyl, and tert-butylaminocarbonyl.

In the context of the invention, di-(C₁-C₆)-alkylaminocarbonyl and di-(C₁-C₄)-alkylaminocarbonyl represent a straight-chain or branched dialkylamino radical having in each case 1 to 6 and 1 to 4 carbon atoms, respectively, which is attached via a carbonyl group. Preference is given to straight-chain or branched dialkylaminocarbonyl radicals having in each case 1 to 4 carbon atoms in the alkylamino group. The following radicals may be mentioned by way of example and by way of preference: N,N-dimethylaminocarbonyl, N,N-diethylaminocarbonyl, N-ethyl-N-methylamino-carbonyl, N-methyl-N-n-propylaminocarbonyl, N-isopropyl-N-n-propylaminocarbonyl, N-tert-butyl-N-methylaminocarbonyl, N-ethyl-N-n-pentylaminocarbonyl and N-n-hexyl-N-methylamino-carbonyl.

In the context of the invention, (C₁-C₆)-alkylthio and (C₁-C₄)-alkylthio represent a straight-chain or branched alkylthio radical having 1 to 6 and 1 to 4 carbon atoms, respectively. Preference is given to a straight-chain or branched alkylthio radical having 1 bis 4 carbon atoms. The following radicals may be mentioned by way of example and by way of preference: methylthio, ethylthio, n-propylthio, isopropylthio, n-butylthio, tert-butylthio, n-pentylthio and n-hexylthio.

In the context of the invention, (C₁-C₆)-alkylsulfonyl represents a straight-chain or branched alkyl-sulfonyl radical having 1 to 6 carbon atoms. Preference is given to a straight-chain or branched alkylsulfonyl radical having 1 to 4 carbon atoms. The following radicals may be mentioned by way of example and by way of preference: methylsulfonyl, ethylsulfonyl, n-propylsulfonyl, isopropylsulfonyl, n-butylsulfonyl, tert-butylsulfonyl, n-pentylsulfonyl and n-hexylsulfonyl.

In the context of the invention, 5- or 6-membered heteroarylene represents a bivalent monocyclic aromatic heterocycle (heteroaromatic) having a total of 5 or 6 ring atoms and up to three identical or different ring heteroatoms from the group consisting of N, O and S which is attached via adjacent ring carbon atoms or, if appropriate, ring nitrogen atoms. The following radicals may be mentioned by way of example: furylene, pyrrolylene, thienylene, pyrazolylene, imidazolylene, thiazolylene, oxazolylene, isoxazolylene, isothiazolylene, triazolylene, oxadiazolylene, thiadiazolylene, pyridylene, pyrimidinylene, pyridazinylene, pyrazinylene. Preference is given to 5- or 6-membered heteroarylene groups having up to two heteroatoms from the group consisting of N, O and S, such as, for example furylene, pyrrolylene, thienylene, thiazolylene, oxazolylene, isoxazolylene, isothiazolylene, imidazolylene, pyrazolylene, pyridylene, pyrimidinylene, pyridazinylene, pyrazinylene.

If radicals in the compounds according to the invention are substituted, the radicals can, unless specified otherwise, be mono- or polysubstituted. In the context of the present invention, the meanings of radicals which occur more than once are independent of one another. Substitution with one, two or three identical or different substituents is preferred. Very particular preference is given to substitution with one substituent.

A particular embodiment of the invention comprises compounds of the formula (I) in which
n represents the number 1, 2 or 3,
R¹ represents hydrogen, hydroxyl, (C₁-C₄)-alkyl, (C₁-C₄)-alkanoyl or cyano,
R² and R³ are identical or different and independently of one another represent hydrogen, fluorine, chlorine, cyano, (C₁-C₄)-alkyl, cyclopropyl, trifluoromethyl, hydroxyl, (C₁-C₄)-alkoxy, trifluoromethoxy, amino, mono- or di-(C₁-C₄)-alkylamino,
A represents a phenylene- or a 5- or 6-membered heteroarylene ring where the two groupings —CO—NH-phenyl and —NH—CO-Z are located at adjacent ring atoms of the phenylene or heteroarylene ring and where phenylene and heteroarylene may additionally be substituted by substituents selected from the group consisting of fluorine, chlorine, cyano, (C₁-C₆)-alkyl, (C₃-C₇)-cycloalkyl, trifluoromethyl, hydroxyl, (C₁-C₆)-alkoxy, trifluoromethoxy, amino, mono- and di-(C₁-C₆)-alkylamino, (C₃-C₇)-cycloalkylamino, (C₁-C₆)-alkanoylamino, (C₁-C₆)-alkoxycarbonylamino, hydroxycarbonyl, (C₁-C₆)-alkoxycarbonyl, aminocarbonyl, mono- and di-(C₁-C₆)-alkylaminocarbonyl,
   where (C₁-C₆)-alkyl, (C₁-C₆)-alkoxy, mono- and di-(C₁-C₆)-alkylamino for their part may each be substituted by hydroxyl, (C₁-C₄)-alkoxy, amino, mono- or di-(C₁-C₄)-alkylamino or (C₃-C₅)-cycloalkylamino,
      and
Z represents phenyl, pyridyl, pyrimidinyl, pyrazinyl or thienyl which may in each case be mono- or disubstituted by identical or different substituents selected from the group consisting of fluorine, chlorine, cyano, methoxy, (C₁-C₄)-alkyl (which for its part may be substituted by amino), ethynyl and amino,
   and salts, solvates and solvates of the salts thereof.

Preference is given to compounds of the formula (I) in which
n represents the number 1 or 2,
R¹ represents hydrogen,
R² represents hydrogen and
R³ represents hydrogen, fluorine or methyl.

Preference is also given to compounds of the formula (I) in which
A represents a group of the formula

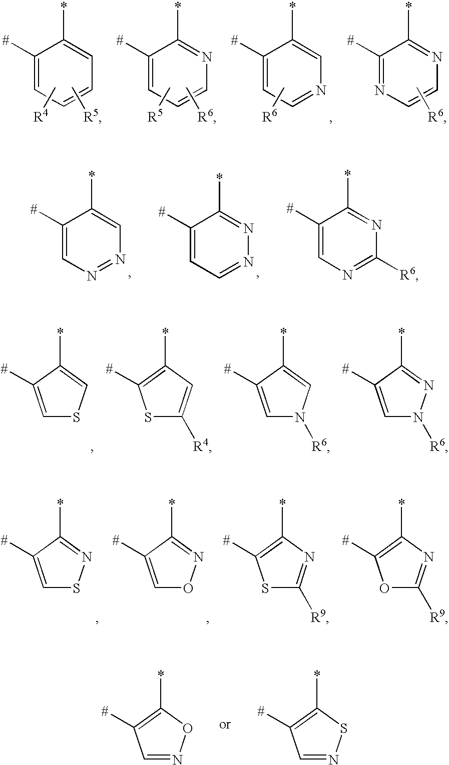

in which
R⁴ represents hydrogen, fluorine, chlorine, cyano, (C₁-C₆)-alkyl, trifluoromethyl, (C₃-C₇)-cycloalkyl, aminocarbonyl, mono- or di-(C₁-C₆)-alkylaminocarbonyl,
R⁵ represents hydrogen, fluorine, chlorine, cyano, (C₁-C₆)-alkyl, (C₃-C₇)-cycloalkyl, (C₁-C₆)-alkoxy, trifluoromethoxy, hydroxyl, amino, mono- or di-(C₁-C₆)-alkylamino, (C₃-C₇)-cycloalkylamino, (C₁-C₆)-alkanoylamino, (C₁-C₆)-alkoxy-carbonylamino, hydroxycarbonyl or aminocarbonyl,
   where (C₁-C₆)-alkyl, (C₁-C₆)-alkoxy, mono- and di-(C₁-C₆)-alkylamino for their part may each be substituted by hydroxyl, (C₁-C₄)-alkoxy, amino, mono- or di-(C₁-C₄)-alkylamino or (C₃-C₅)-cycloalkylamino,
R⁶ represents hydrogen, (C₁-C₆)-alkyl or (C₃-C₇)-cycloalkyl,
R⁹ represents hydrogen, (C₁-C₆)-alkyl, (C₃-C₇)-cycloalkyl, (C₁-C₆)-alkylthio or (C₁-C₆)-alkylsulfonyl
and
# and * represent the points of attachment to the —CO—NH-phenyl- and the —NH—CO-Z grouping.

Here, a particular embodiment of the invention comprises compounds of the formula (I) in which
A represents a group of the formula

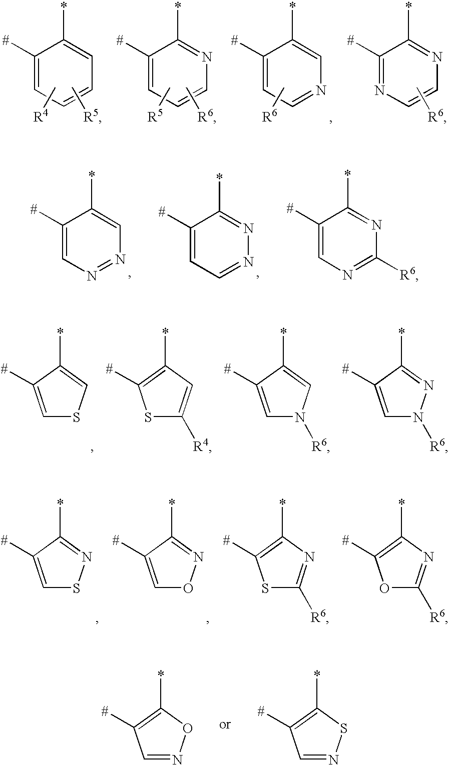

in which
R⁴ represents hydrogen, fluorine, chlorine, cyano, (C₁-C₆)-alkyl, trifluoromethyl, (C₃-C₇)-cycloalkyl, aminocarbonyl, mono- or di-(C₁-C₆)-alkylaminocarbonyl,
R⁵ represents hydrogen, fluorine, chlorine, cyano, (C₁-C₆)-alkyl, (C₃-C₇)-cycloalkyl, (C₁-C₆)-alkoxy, trifluoromethoxy, hydroxyl, amino, mono- or di-(C₁-C₆)-alkylamino, (C₃-C₇)-cycloalkylamino, (C₁-C₆)-alkanoylamino, (C₁-C₆)-alkoxy-carbonylamino, hydroxylcarbonyl or aminocarbonyl,
   where (C₁-C₆)-alkyl, (C₁-C₆)-alkoxy, mono- and di-(C₁-C₆)-alkylamino for their part may each be substituted by hydroxyl, (C₁-C₄)-alkoxy, amino, mono- or di-(C₁-C₄)-alkylamino or (C₃-C₅)-cycloalkylamino,
R⁶ represents hydrogen, (C₁-C₆)-alkyl or (C₃-C₇)-cycloalkyl
and
# and * represent the points of attachment to the —CO—NH-phenyl- and the —NH—CO-Z grouping.

Preference is furthermore given to compounds of the formula (I) in which
Z represents a group of the formula

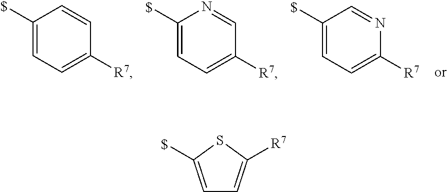

in which
R⁷ represents fluorine, chlorine, methyl or ethynyl
and
$ represents the point of attachment to the carbonyl group.

Particular preference is given to compounds of the formula (I) in which
n represents the number 1 or 2,
R¹ represents hydrogen,
R² represents hydrogen,
R³ represents hydrogen, fluorine or methyl,
A represents a group of the formula

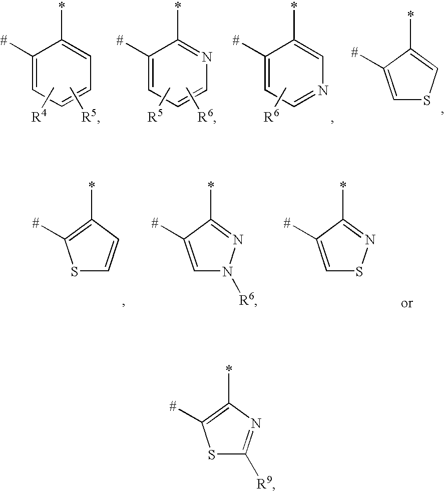

in which
R⁴ represents hydrogen, fluorine, chlorine, cyano, (C₁-C₄)-alkyl, trifluoromethyl, aminocarbonyl or di-(C₁-C₄)-alkylaminocarbonyl,
R⁵ represents hydrogen, fluorine, cyano, (C₁-C₄)-alkyl, (C₁-C₄)-alkoxy or mono- or di-(C₁-C₄)-alkylamino,
R⁶ represents hydrogen, (C₁-C₄)-alkyl or (C₃-C₅)-cycloalkyl,
R⁹ represents hydrogen, (C₁-C₄)-alkyl, (C₃-C₅)-cycloalkyl, (C₁-C₄)-alkylthio or (C₁-C₄)-alkylsulfonyl
and
# and * represent the points of attachment to the —CO—NH-phenyl- and the —NH—CO-Z grouping,
   and
Z represents a group of the formula

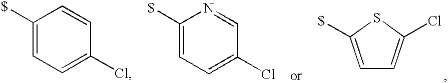

in which $ represents the point of attachment to the carbonyl group, and salts, solvates and solvates of the salts thereof.

Here, a particular embodiment of the invention comprises compounds of the formula (I) in which
n represents the number 1 or 2,
R¹ represents hydrogen,
R² represents hydrogen,
R³ represents hydrogen, fluorine or methyl,
A represents a group of the formula

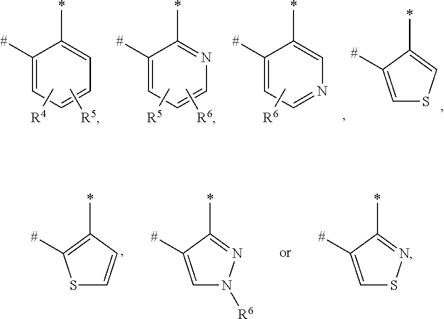

in which
R⁴ represents hydrogen, fluorine, chlorine, cyano, (C₁-C₄)-alkyl, trifluoromethyl, aminocarbonyl or di-(C₁-C₄)-alkylaminocarbonyl,
R⁵ represents hydrogen, fluorine, cyano, (C₁-C₄)-alkyl, (C₁-C₄)-alkoxy or mono- or di-(C₁-C₄)-alkylamino,
R⁶ represents hydrogen, (C₁-C₄)-alkyl or (C₃-C₅)-cycloalkyl
and
# and * represent the points of attachment to the —CO—NH-phenyl- and the —NH—CO-Z grouping,
   and
Z represents a group of the formula

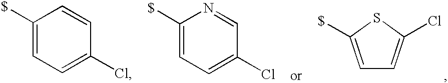

in which $ represents the point of attachment to the carbonyl group, and salts, solvates and solvates of the salts thereof.

Very particular preference is given to the compounds of the formula (I) in which
n represents the number 1 or 2,
R¹ represents hydrogen,
R² represents hydrogen,
R³ represents hydrogen, fluorine or methyl,
A represents a group of the formula

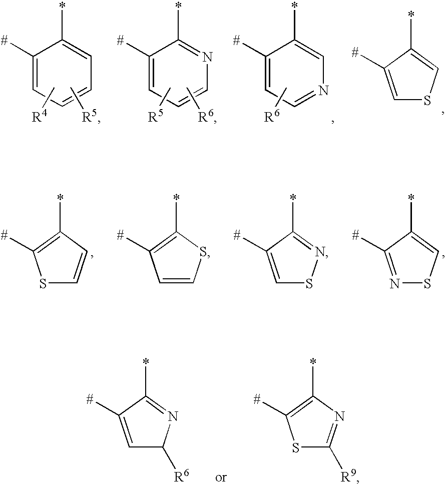

in which
R⁴ represents hydrogen, fluorine, chlorine, cyano, (C₁-C₄)-alkyl, trifluoromethyl, aminocarbonyl or di-(C₁-C₄)-alkylaminocarbonyl,
R⁵ represents hydrogen, fluorine, cyano, (C₁-C₄)-alkyl, (C₁-C₄)-alkoxy or mono- or di-(C₁-C₄)-alkylamino,
R⁶ represents hydrogen, (C₁-C₄)-alkyl or (C₃-C₅)-cycloalkyl,
R⁹ represents hydrogen, (C₁-C₄)-alkyl, (C₃-C₅)-cycloalkyl, (C₁-C₄)-alkylthio or (C₁-C₄)-alkylsulfonyl,
# represents the point of attachment to the —CO—NH-phenyl grouping
and
* represents the point of attachment to the —NH—CO-Z grouping,
   and
Z represents a group of the formula

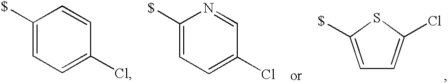

in which $ represents the point of attachment to the carbonyl group,
   and salts, solvates and solvates of the salts thereof.

The particular radical definitions given in the respective combinations or preferred combinations of radicals can be replaced by any radical definitions of other combinations, independently of the respective given combinations of the radicals.

Very particular preference is given to combinations of two or more of the preferred ranges mentioned above.

The invention furthermore provides a process for preparing the compounds of the formula (I) according to the invention in which R¹ represents hydrogen, characterized in that compounds of the formula (II)

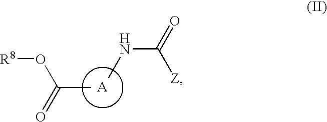

in which A and Z have the meanings given above and
R⁸ represents hydrogen, methyl or ethyl,
   are initially converted in an inert solvent with activation of the ester or the carboxylic acid function with a compound of the formula (III)

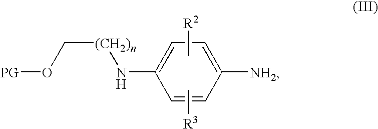

in which n, R² and R³ have the meanings given above
and
PG represents a hydroxyl protective group, preferably trimethylsilyl or tert-butyldimethylsilyl, into compounds of the formula (IV)

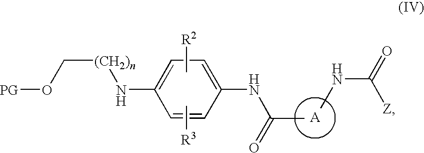

in which n, A, PG, Z, R² and R³ have the meanings given above, then either
[A] by removal of the protective group PG under customary conditions converted into compounds of the formula (V)

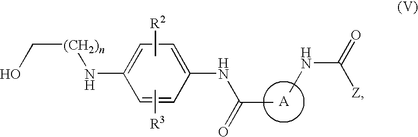

in which n, A, Z, R² and R³ have the meanings given above,
and the compounds of the formula (V) are then in an inert solvent in the presence of an acid converted with cyanogen bromide into compounds of the formula (I-A)

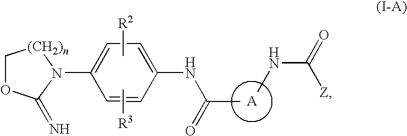

in which n, A, Z, R² and R³ have the meanings given above, or
[B] initially in an inert solvent converted with cyanogen bromide, preferably in the presence of a base, into compounds of the formula (VI)

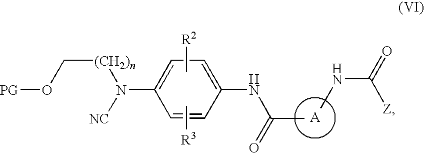

in which n, A, PG, Z, R² and R³ have the meanings given above,
then by removal of the protective group PG converted into compounds of the formula (VII)

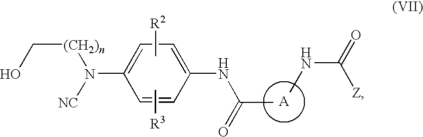

in which n, A, Z, R² and R³ have the meanings given above,
and the compounds of the formula (VII) are then cyclized in an inert solvent in the presence of an acid to compounds of the formula (I-A)
   and the compounds of the formula (I-A) are, if appropriate, converted with the appropriate (i) solvents and/or (ii) bases or acids into their solvates, salts and/or solvates of the salts.

The compounds of the formula (I) according to the invention in which R¹ does not represent hydrogen can be prepared from the compounds of the formula (V) analogously to processes known from the literature [cf., for example, for R¹=alkanoyl: D. Douglass, J. Amer. Chem. Soc. 1934, 56, 719 and T. Shibanuma, M. Shiono, T. Mukaiyama, Chem. Lett. 1977, 575-576; for R¹=cyano: a) R. Evers, M. Michalik, J. Prakt. Chem. 1991, 333, 699-710; N. Maezaki, A. Furusawa, S. Uchida, T. Tanaka, Tetrahedron 2001, 57, 9309-9316; G. Berecz, J. Reiter, G. Argay, A. Kalman, J. Heterocycl. Chem. 2002, 39, 319-326; b) R. Mohr, A. Buschauer, W. Schunack, Arch. Pharm. (Weinheim Ger.) 1988, 321, 221-227; for R¹=alkyl: a) V. A. Vaillancourt et al., J. Med. Chem. 2001, 44, 1231-1248; b) F. B. Dains et al., J. Amer. Chem. Soc. 1925, 47, 1981-1989; J. Amer. Chem. Soc. 1922, 44, 2637-2643 and T. Shibanuma, M. Shiono, T. Mukaiyama, Chem. Lett. 1977, 575-576].

Inert solvents for the process step (II)+(III)→(IV) are, for example, ethers, such as diethylether, dioxane, tetrahydrofuran, glycol dimethyl ether or diethylene glycol dimethyl ether, hydrocarbons, such as benzene, toluene, xylene, hexane, cyclohexane or mineral oil fractions, halogenated hydrocarbons, such as dichloromethane, trichloromethane, carbon tetrachloride, 1,2-dichloro-ethane, trichloroethylene or chlorobenzene, or other solvents, such as ethyl acetat, pyridine, dimethyl sulfoxide, dimethylformamide, N,N′-dimethylpropyleneurea (DMPU), N-methyl-pyrrolidone (NMP), acetonitrile or acetone. It is also possible to use mixtures of these solvents. Preference is given to dichloromethane, tetrahydrofuran, dimethylformamide or mixtures of these solvents.

The preferred activating agent for the amide formation from carboxylic esters in process step (II)+(III)→(IV) [R⁸ in (II)=methyl or ethyl] is trimethylaluminum. The reaction is preferably carried out in a temperature range of from 0° C. to +40° C. The reaction can be carried out under atmospheric, elevated or reduced pressure (for example from 0.5 to 5 bar). In general, the reaction is carried out under atmospheric pressure.

Suitable condensing agents for the amide formation from carboxylic acids in process step (II)+(III)→(IV) [R⁸ in (II)=hydrogen] are, for example carbodiimides, such as N,N′-diethylcarbodiimide, N,N′-dipropylcarbodiimide, N,N′-diisopropylcarbodiimide, N,N′-dicyclohexylcarbodiimide (DCC), N-(3-dimethylaminoisopropyl)-N′-ethylcarbodiimide hydrochloride (EDC), or phosgene derivatives, such as N,N′-carbonyldiimidazole, or 1,2-oxazolium compounds, such as 2-ethyl-5-phenyl-1,2-oxazolium 3-sulfate or 2-tert-butyl-5-methylisoxazolium perchlorate, or acylamino compounds, such as 2-ethoxy-1-ethoxycarbonyl-1,2-dihydroquinoline, or isobutyl chloroformate, propanephosphonic anhydride, diethyl cyanophosphonate, bis-(2-oxo-3-oxazolidinyl)phosphoryl chloride, benzotriazol-1-yloxy-tris(dimethylamino)phosphonium hexafluorophosphate, benzo-triazol-1-yloxy-tris(pyrrolidino)phosphonium hexafluorophosphate (PyBOP), O-(benzotriazol-1-yl)-N,N,N′N′-tetramethyluronium tetrafluoroborate (TBTU), O-(benzotriazol-1-yl)-N,N,N′,N′-tetramethyluronium hexafluorophosphate (HBTU), 2-(2-oxo-1-(2H)-pyridyl)-1,1,3,3-tetramethyl-uronium tetrafluoroborate (TPTU), O-(7-azabenzotriazol-1-yl)-N,N,N′,N′-tetramethyluronium hexafluorophosphate (HATU) or O-(1H-6-chlorobenzotriazol-1-yl)-1,1,3,3-tetramethyluronium tetrafluoroborate (TCTU), if appropriate in combination with further auxiliaries, such as 1-hydroxybenzotriazole (HOBt) or N-hydroxysuccinimide (HOSu), and also as bases alkali metal carbonates, for example sodium carbonate or potassium carbonate or sodium bicarbonate or potassium bicarbonate, or organic bases, such as trialkylamines, for example triethylamine, N-methylmorpholine, N-methylpiperidine or N,N-diisopropylethylamine. Preference is given to using TBTU in combination with N,N-diisopropylethylamine.

The condensation (II)+(III)→(IV) [R⁸ in (II)=hydrogen] is generally carried out in a tempera-ture range of from −20° C. to +60° C., preferably from 0° C. to +40° C. The reaction can be carried out under atmospheric, elevated or reduced pressure (for example from 0.5 to 5 bar). In general, the reaction is carried out under atmospheric pressure.

In process steps (IV)→(V) and (VI)→(VII), the removal of trimethylsilyl or tert-butyldimethylsilyl as preferred hydroxylprotective groups (PG) is preferably carried out with the aid of tetra-n-butylammonium fluoride (TBAF) or, in the case of the reaction (IV)→(V), also with hydrogen fluoride. The reactions are generally carried out in tetrahydrofuran as solvent in a temperature range of from 0° C. to +40° C.

The reaction sequence (VI)→(VII)→(I-A) is altogether particularly preferably carried out using an acid-labile hydroxylprotective group, such as, for example, trimethylsilyl or tert-butyldimethyl-silyl, in the presence of an excess of an acid as a one-pot reaction, without isolation of the intermediate (VII).

Suitable inert solvents for the process steps (V)→(I-A), (IV)→(VI) and (VII)→(I-A) are in particular tetrahydrofuran, dichloromethane or acetonitrile or mixtures of these solvents. These process steps are generally carried out in a temperature range of from −20° C. to +50° C., preferably from 0° C. to +40° C. The reactions can be carried out under atmospheric, elevated or reduced pressure (for example from 0.5 to 5 bar). In general, the reactions are carried out under atmospheric pressure.

Suitable acids for the process steps (V)→(I-A) and (VII)→(I-A) and the reaction sequence (VI)→(VII)→(I-A) are in particular strong inorganic or organic acids, such as, for example, hydrogen fluoride, hydrogen chloride, hydrogen bromide, methanesulfonic acid, trifluoro-methanesulfonic acid or trifluoroacetic acid.

The process step (IV)→(VI) is preferably carried out in the presence of a base. Suitable for this purpose are in particular inorganic bases, such as, for example, alkali metal or alkaline earth metal carbonates or bicarbonates, such as lithium carbonate, sodium carbonate, potassium carbonate, calcium carbonate or cesium carbonate or sodium bicarbonate or potassium bicarbonate, or alkali metal hydrides, such as sodium hydride.

The compounds of the formula (II) can be obtained by processes customary in the literature, for example by reacting a compound of the formula (VIII)

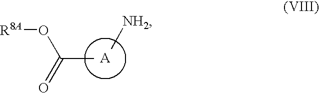

in which A has the meaning given above and
R^{8A} represents methyl or ethyl,
with a compound of the formula (IX)

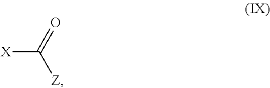

in which Z has the meaning given above and
X represents hydroxyl or a leaving group, such as, for example, chlorine or bromine, and—if R⁸ in (II) represents hydrogen—subsequent hydrolysis of the ester grouping —COOR^{8A}.

The compounds of the formula (III) can be obtained analogously to processes known from the literature, for example by reacting compounds of the formula (X)

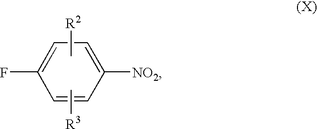

in which R² and R³ have the meanings given above,
with compounds of the formula (XI)

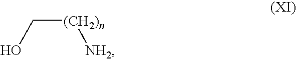

in which n has the meaning given above,
to give compounds of the formula (XII)

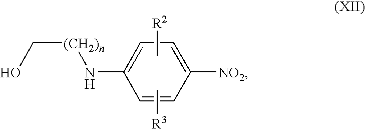

in which n, R² and R³ have the meanings given above,
subsequent introduction of the hydroxyl protective group PG and subsequent reduction of the nitro group to the amine.

The compounds of the formulae (VIII), (IX), (X) and (XI) are commercially available, known from the literature or can be prepared analogously to processes known from the literature.

The preparation of the compounds according to the invention can be illustrated by the synthesis scheme below:

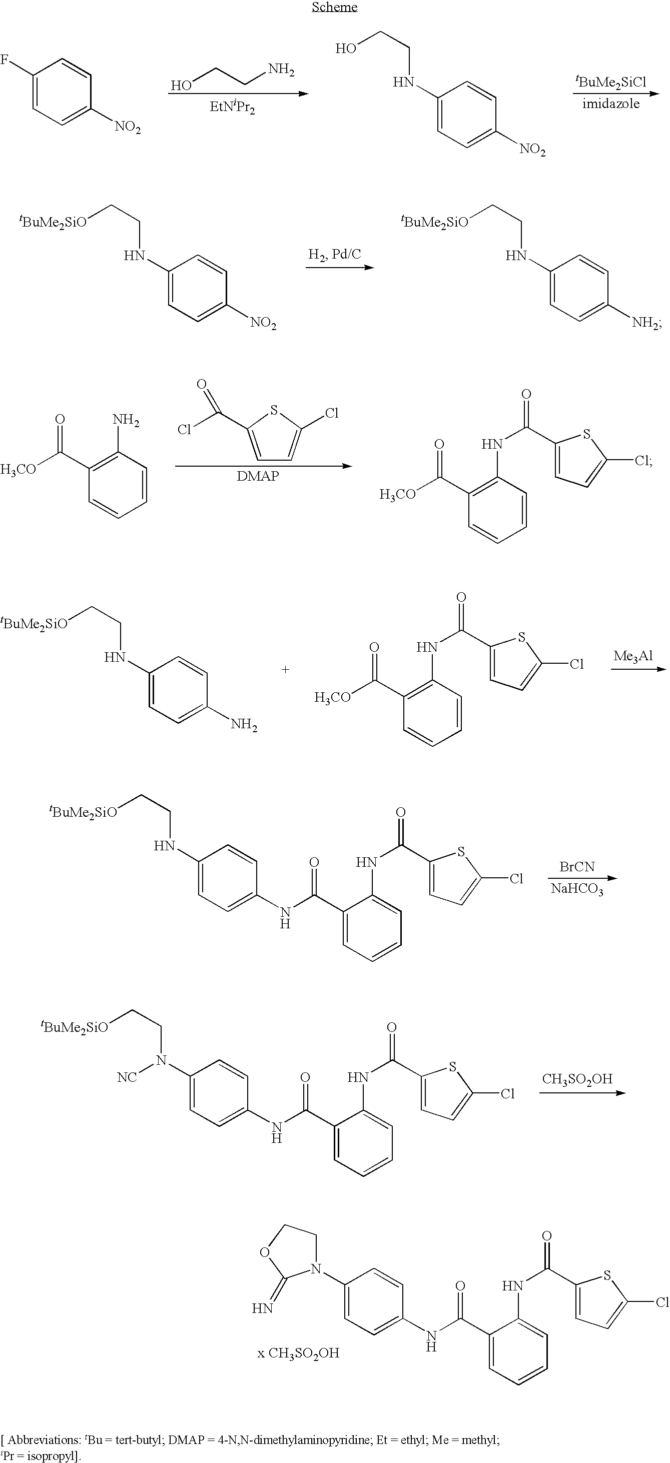

The compounds according to the invention have an unforeseeable useful pharmacological activity spectrum. Accordingly, they are suitable for use as medicaments for the treatment and/or prophylaxis of diseases in humans and animals.

The compounds according to the invention are selective inhibitors of blood coagulation factor Xa which act in particular as anticoagulants.

In addition, the compounds according to the invention have favorable physicochemical properties, such as, for example, good solubility in water and physiological media, which is advantageous for their therapeutic application.

The present invention furthermore provides the use of the compounds according to the invention for the treatment and/or prophylaxis of disorders, preferably thromboembolic disorders and/or thromboembolic complications.

For the purposes of the present invention, “thromboembolic disorders” include in particular disorders such as ST-elevation myocardial infarction (STEMI) or non-STelevation myocardial infarction (non-STEMI), stable angina pectoris, unstable angina pectoris, reocclusions and restenoses after coronary interventions such as angioplasty or aortocoronary bypass, peripheral areterial occlusive diseases, pulmonary embolisms, deep vein thromboses and kidney vein thromboses, transitory ischemic attacks and also thrombotic and thromboembolic stroke.

Accordingly, the substances are also suitable for preventing and treating cardiogenic thrombo-embolisms, such as, for example, brain ischemias, stroke and systemic thromboembolisms and ischemias, in patients having acute, intermittent or persistent cardioarrhythmias, such as, for example, atrial fibrillation, and those undergoing cardioversion, furthermore patients having heart valve disorders or having artificial heart valves. In addition, the compounds according to the invention are suitable for treating disseminated intravascular coagulation (DIC).

Thromboembolic complications furthermore occur during microangiopathic hemolytic anemias, extracorporeal blood circulation, such as hemodialysis, and in connection with heart valve prostheses.

Moreover, the compounds according to the invention are also suitable for the prophylaxis and/or treatment of atherosclerotic vascular disorders and inflammatory disorders, such as rheumatic disorders of the locomotor apparatus, and in addition also for the prophylaxis and/or treatment of Alzheimer's disease. Moreover, the compounds according to the invention can be used for inhibiting tumour growth and formation of metastases, for microangiopathies, age-related macular degeneration, diabetic retinopathy, diabetic nephropathy and other microvascular disorders, and also for the prevention and treatment of thromboembolic complications, such as, for example, venous thromboembolisms, in tumour patients, in particular patients undergoing major surgical interventions or chemo- or radiotherapy.

The compounds according to the invention can additionally also be used for preventing coagulation ex vivo, for example for preserving blood and plasma products, for cleaning/pretreating catheters and other medical tools and instruments, for coating synthetic surfaces of medical tools and instruments used in vivo or ex vivo or for biological samples comprising factor Xa.

The present invention furthermore provides the use of the compounds according to the invention for the treatment and/or prophylaxis of disorders, in particular the disorders mentioned above.

The present invention furthermore provides the use of the compounds according to the invention for preparing a medicament for the treatment and/or prophylaxis of disorders, in particular the disorders mentioned above.

The present invention furthermore provides a method for the treatment and/or prophylaxis of disorders, in particular the disorders mentioned above, using an anticoagulatory effective amount of the compound according to the invention.

The present invention furthermore provides a method for preventing blood coagulation in vitro, in particular in banked blood or biological samples comprising factor Xa, which method is characterized in that an anticoagulatory effective amount of the compound according to the invention is added.

The present invention furthermore provides medicaments comprising a compound according to the invention and one or more further active compounds, in particular for the treatment and/or prophylaxis of the disorders mentioned above. The following compounds may be mentioned by way of example and by way of preference as active compounds suitable for combinations:
lipid-lowering agents, in particular HMG-CoA (3-hydroxy-3-methylglutaryl-coenzyme A) reductase inhibitors;
coronary therapeutics/vasodilators, in particular ACE (angiotensin converting enzyme) inhibitors; AII (angiotensin II) receptor antagonists; β-adrenoceptor antagonists; alpha-1-adrenoceptor antagonists; diuretics; calcium channel blockers; substances which cause an increase in the cyclic guanosine monophosphate (cGMP) concentration such as, for example, stimulators of soluble guanylate cyclase;
plasminogen activators (thrombolytics/fibrinolytics) and compounds enhancing thrombolysis/fibrinolysis, such as inhibitors of the plasminogen activator inhibitor (PAI inhibitors) or inhibitors of the thrombin-activated fibrinolysis inhibitor (TAFI inhibitors);
anticoagulants;
platelet aggregation inhibiting substances (platelet aggregation inhibitors, thrombocyte aggregation inhibitors);
fibrinogen receptor antagonists (glycoprotein-IIb/IIIa antagonists);
and also antiarrhythmics.

The present invention furthermore provides medicaments comprising at least one compound according to the invention, usually together with one or more inert nontoxic pharmaceutically acceptable auxiliaries, and their use for the purposes mentioned above.

The compounds according to the invention can act systemically and/or locally. For this purpose, they can be administered in a suitable way, such as, for example, by the oral, parenteral, pulmonary, nasal, sublingual, lingual, buccal, rectal, dermal, transdermal, conjunctival or otic route, or as implant or stent.

For these administration routes, it is possible to administer the compounds according to the invention in suitable administration forms.

Suitable for oral administration are administration forms which work as described in the prior art and deliver the compounds according to the invention rapidly and/or in modified form, which comprise the compounds according to the invention in crystalline and/or amorphous and/or dissolved form, such as, for example, tablets (uncoated and coated tablets, for example tablets provided with enteric coatings or coatings whose dissolution is delayed or which are insoluble and which control the release of the compound according to the invention), tablets which rapidly decompose in the oral cavity, or films/wafers, films/lyophilizates, capsules (for example hard or soft gelatin capsules), sugar-coated tablets, granules, pellets, powders, emulsions, suspensions, aerosols or solutions.

Parenteral administration can take place with avoidance of an absorption step (for example intravenously, intraarterially, intracardially, intraspinally or intralumbarly) or with inclusion of absorption (for example intramuscularly, subcutaneously, intracutaneously, percutaneously or intraperitoneally). Administration forms suitable for parenteral administration are, inter alia, preparations for injection and infusion in the form of solutions, suspensions, emulsions, lyophilizates or sterile powders.

Examples suitable for other administration routes are pharmaceutical forms for inhalation (inter alia powder inhalers, nebulizers), nasal drops/solutions/sprays; tablets to be administered lingually, sublingually or buccally, films/wafers or capsules, suppositories, preparations for the eyes or ears, vaginal capsules, aqueous suspensions (lotions, shaking mixtures), lipophilic suspensions, ointments, creams, transdermal therapeutic systems, (e.g. patches), milk, pastes, foams, dusting powders, implants or stents.

Preference is given to oral or parenteral administration, in particular oral administration.

The compounds according to the invention can be converted into the stated administration forms. This can take place in a manner known per se by mixing with inert, nontoxic, pharmaceutically suitable auxiliaries. These auxiliaries include, inter alia, carriers (for example microcrystalline cellulose, lactose, mannitol), solvents (for example liquid polyethylene glycols), emulsifiers and dispersants or wetting agents (for example sodium dodecyl sulfate, polyoxysorbitan oleate), binders (for example polyvinylpyrrolidone), synthetic and natural polymers (for example albumin), stabilizers (for example antioxidants, such as, for example, ascorbic acid), colorants (for example inorganic pigments, such as, for example, iron oxides) and flavor- and/or odor-masking agents.

In general, it has proved advantageous to administer on parenteral administration amounts of from about 0.001 to 1 mg/kg, preferably from about 0.01 to 0.5 mg/kg, of body weight to achieve effective results. The dosage on oral administration is from about 0.01 to 100 mg/kg, preferably about 0.01 to 20 mg/kg, and very particularly preferably 0.1 to 10 mg/kg, of body weight.

It may nevertheless be necessary, where appropriate, to deviate from the amounts mentioned, depending on the body weight, the administration route, the individual response to the active compound, the mode of preparation and the time or interval over which administration takes place. Thus, in some cases it may be sufficient to make do with less than the aforementioned minimal amount, whereas in other cases the upper limit mentioned must be exceeded. In the event of administration of larger amounts, it may be advisable to divide these into a plurality of individual doses over the day.

The invention is illustrated by the working examples below. The invention is not limited to the examples.

The percentage data in the following tests and examples are percentages by weight unless otherwise indicated; parts are parts by weight. Solvent ratios, dilution ratios and concentration data of liquid/liquid solutions are in each case based on volume.

### A. EXAMPLES

### Abbreviations and Acronyms

TLC thin-layer chromatography
DCI direct chemical ionisation (in MS)
DMF N,N-dimethylformamide
DMSO dimethyl sulfoxide
d day(s)
ee enantiomeric excess
eq. equivalent(s)
ESI electrospray ionisation (in MS)
h hour(s)
HPLC high pressure, high performance liquid chromatography
LC-MS liquid chromatography-coupled mass spectroscopy
min minute(s)
MS mass spectroscopy
NMR nuclear magnetic resonance spectroscopy
RP reverse phase (in HPLC)
RT room temperature
Rₜ retention time (in HPLC)
TBTU O-(benzotriazol-1-yl)-N,N,N,N′-tetramethyluronium tetrafluoroborate
THF tetrahydrofuran

### LC-MS and HPLC Methods:

### Method 1:

MS instrument type: Micromass ZQ; HPLC instrument type: Waters Alliance 2795; column: Phenomenex Synergi 2μ Hydro-RP Mercury 20 mm×4 mm; mobile phase A: 1 l of water+0.5 ml of 50% strength formic acid, mobile phase B: 1 l of acetonitrile+0.5 ml of 50% strength formic acid; gradient: 0.0 min 90% A→2.5 min 30% A→3.0 min 5% A→4.5 min 5% A; flow rate: 0.0 min 1 ml/min, 2.5 min/3.0 min/4.5 min 2 ml/min; oven: 50° C.; UV detection: 210 nm.

### Method 2:

MS instrument type: Micromass ZQ; HPLC instrument type: HP 1100 Series; UV DAD; column: Phenomenex Synergi 2μ Hydro-RP Mercury 20 mm×4 mm; mobile phase A: 1 l of water+0.5 ml of 50% strength formic acid, mobile phase B: 1 l of acetonitrile+0.5 ml of 50% strength formic acid; gradient: 0.0 min 90% A→2.5 min 30% A 3.0 min 5% A→4.5 min 5% A; flow rate: 0.0 min 1 ml/min, 2.5 mini/3.0 min/4.5 min 2 ml/min; oven: 50° C.; UV detection: 210 nm.

### Method 3:

Instrument: Micromass Quattro LCZ with HPLC Agilent Series 1100; column: Phenomenex Synergi 2μ Hydro-RP Mercury 20 mm×4 mm; mobile phase A: 1 l of water+0.5 ml of 50% strength formic acid, mobile phase B: 1 l of acetonitrile+0.5 ml of 50% strength formic acid; gradient: 0.0 min 90% A→2.5 min 30% A→3.0 min 5% A→4.5 min 5% A; flow rate: 0.0 min 1 ml/min, 2.5 min/3.0 min/4.5 min 2 ml/min; oven: 50° C.; UV detection: 208-400 nm.

### Method 4:

Instrument: Micromass Platform LCZ with HPLC Agilent Series 1100; column: Phenomenex Synergi 2μ Hydro-RP Mercury 20 mm×4 mm; mobile phase A: 1 l of water+0.5 ml of 50% strength formic acid, mobile phase B: 1 l of acetonitrile+0.5 ml of 50% strength formic acid; gradient: 0.0 min 90% A→2.5 min 30% A→3.0 min 5% A→4.5 min 5% A; flow rate: 0.0 min 1 ml/min, 2.5 min/3.0 min/4.5 min 2 ml/min; oven: 50° C.; UV detection: 210 nm.

### Method 5:

Instrument: Micromass Platform LCZ with HPLC Agilent Series 1100; column: Thermo HyPURITY Aquastar 3μ 50 mm×2.1 mm; mobile phase A: 1 l of water+0.5 ml of 50% strength formic acid, mobile phase B: 1 l of acetonitrile+0.5 ml of 50% strength formic acid; gradient: 0.0 min 100% A→0.2 min 100% A→2.9 min 30% A→3.1 min 10% A→5.5 min 10% A; oven: 50° C.; flow rate: 0.8 ml/min; UV detection: 210 nm.

### Method 6:

MS instrument type: Micromass ZQ; HPLC instrument type: Waters Alliance 2795; column: Merck Chromolith SpeedROD RP-18e 50 mm×4.6 mm; mobile phase A: 1 l of water+0.5 ml of 50% strength formic acid, mobile phase B: 1 l of acetonitrile+0.5 ml of 50% strength formic acid; gradient: 0.0 min 10% B→3.0 min 95% B→4.0 min 95% B; oven: 35° C.; flow rate: 0.0 min 1.0 ml/min→3.0 min 3.0 ml/min→4.0 min 3.0 ml/min; UV detection: 210 nm.

### Method 7:

Instrument: HP 1100 with DAD detection; column: Kromasil 100 RP-18, 60 mm×2.1 mm, 3.5 μm; mobile phase A: 5 ml of HClO₄ (70% strength)/l of water, mobile phase B: acetonitrile; gradient: 0 min 2% B→0.5 min 2% B→4.5 min 90% B→9 min 90% B→9.2 min 2% B→10 min 2% B; flow rate: 0.75 ml/min; column temperature: 30° C.; UV detection: 210 nm.

### Method 8:

Instrument: HP 1100 with DAD detection; column: Kromasil 100 RP-18, 60 mm×2.1 mm, 3.5 μm; mobile phase A: 5 ml of HClO₄ (70% strength)/l of water, mobile phase B: acetonitrile; gradient: 0 min 2% B→0.5 min 2% B→4.5 min 90% B→15 min 90% B→15.2 min 2% B→16 min 2% B; flow rate: 0.75 ml/min; column temperature: 30° C.; UV detection: 210 nm.

### Method 9:

Instrument: HP 1100 with DAD detection; column: Kromasil 100 RP-18, 60 mm×2.1 mm, 3.5 μm; mobile phase A: 5 ml of HClO₄ (70% strength)/l of water, mobile phase B: acetonitrile; gradient: 0 min 2% B→0.5 min 2% B→4.5 min 90% B→6.5 min 90% B→6.7 min 2% B 7.5 min 2% B; flow rate: 0.75 ml/min; column temperature: 30° C.; UV detection: 210 nm.

### Starting Materials and Intermediates:

### General Method I: Esterification of Carboxylic Acids

5 mmol of the carboxylic acid are dissolved in 50 ml of methanol, and 5 ml of concentrated sulfuric acid are added. The mixture is heated under reflux for 14-24 h. After cooling to room temperature, the solution is poured onto ice and adjusted to pH 6 using sodium bicarbonate. After addition of 100 ml of dichloromethane, the aqueous phase is separated off and reextracted twice with in each case 50 ml of dichloromethane. The combined organic extracts are dried over sodium sulfate, filtered and concentrated under reduced pressure.

### General Method II: Acylation of Amino Compounds of the Formula (VIII)

At 55° C., pyridine (2 eq.), 4-N,N-dimethylaminopyridine (0.1 mmol) and a solution of the acid chloride in question (1.5 eq.) in THF (about 1 ml/mmol) are added to a solution of the amino compound in THF (about 2 ml/mmol). The reaction mixture is stirred under reflux for 16 h and then cooled to RT. After addition of water/dichloromethane and phase separation, the aqueous phase is reextracted with dichloromethane. The combined organic phases are washed with water, saturated aqueous sodium bicarbonate solution and saturated aqueous sodium chloride solution, dried over sodium sulfate, filtered and concentrated under reduced pressure. The crude product is either triturated with acetonitrile or purified by flash chromatography on silica gel.

### General Method III: Basic Cleavage of Carboxylic Esters

10 ml of a water/THF mixture (4:1) are added to 1 mmol of the carboxylic ester. 3 mmol of lithium hydroxide monohydrate are added, and the solution is stirred at room temperature for 30 min. After addition of 3 ml (3 mmol) of 1 N hydrochloric acid, the THF is removed under reduced pressure, and the precipitate formed is filtered off and washed with water and then with diethyl ether. The solid is dried under high vacuum.

### Example 1A

### 5-Chlorothiophene-2-carbonyl chloride

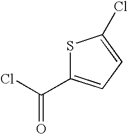

The title compound is prepared by reacting 5-chlorothiophene-2-carboxylic acid with thionyl chloride, see R. Aitken et al., Arch. Pharm. (Weinheim Ger.) 1998, 331, 405-411.

### Example 2A

### 5-Chloropyridine-2-carbonyl chloride

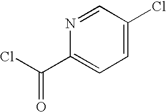

The title compound is prepared by reacting 5-chloropyridine-2-carboxylic acid with thionyl chloride, see Graf et al., J. Prakt. Chem. 1932, 133, 3649.

### Example 3A

### N-(2-{[tert-Butyl(dimethyl)silyl]oxy}ethyl)benzene-1,4-diamine

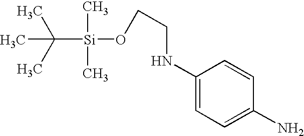

### Step a): 2-[(4-Nitrophenyl)amino]ethanol

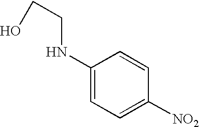

130 ml (2.15 mol, 3 eq.) of 2-aminoethanol and 274 ml (1.57 mol, 2.2 eq.) of N,N-diisopropylethylamine are added to a solution of 101 g (716 mmol) of 4-fluoronitrophenol in 500 ml of ethanol. The reaction mixture is stirred at 50° C. overnight, a further 86 ml (1.43 mol, 2.0 eq.) of 2-aminoethanol and 249 ml (1.43 mol, 2.0 eq.) of N,N-diisopropylethylamine are then added and the mixture is again stirred at 50° C. for 12 h. The reaction solution is then concentrated under reduced pressure and the residue is triturated with 600 ml of water. The precipitate formed is filtered off, washed repeatedly with water and dried.

Yield: 127 g (97% of theory)

LC-MS (Method 5): Rₜ=2.32 min;

MS (ESIpos): m/z=183 [M+H]⁺;

¹H-NMR (300 MHz, DMSO-d₆): δ=7.99 (d, 2H), 7.30 (t, 1H), 6.68 (d, 2H), 4.82 (t, 1H), 3.63-3.52 (m, 2H), 3.30-3.19 (m, 2H).

### Step b): N-(2-{[tert-Butyl(dimethyl)silyl]oxy}ethyl)-4-nitroaniline

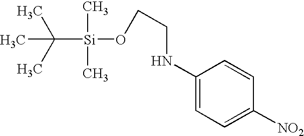

At RT, 30.6 g (203 mmol, 1.2 eq.) of tert-butyldimethylchlorosilane and 17.3 g (254 mmol, 1.5 eq.) of imidazole are added to a solution of 30.8 g (169 mmol) of 2-[(4-nitrophenyl)amino]ethanol in 300 ml of DMF, and the mixture is stirred at RT for 2.5 h. The reaction mixture is concentrated under reduced pressure, and the residue is dissolved in 200 ml of dichloromethane and 100 ml of water. After phase separation, the aqueous phase is extracted three times with in each case 80 ml of dichloromethane. The combined organic phases are washed with 100 ml of saturated aqueous sodium chloride solution, dried over sodium sulfate, filtered and concentrated under reduced pressure.

Yield: 49.7 g (quant.)

LC-MS (Method 3): Rₜ=3.09 min;

MS (ESIpos): m/z=297 [M+H]⁺;

¹H-NMR (300 MHz, DMSO-d₆): δ=7.98 (d, 2H), 7.29 (t, 1H), 6.68 (d, 2H), 3.77-3.66 (m, 2H), 3.35-3.24 (m, 2H), 0.81 (s, 9H), 0.0 (s, 6H).

### Step c): N-(2-{[tert-Butyl(dimethyl)silyl]oxy}ethyl)benzene-1,4-diamine

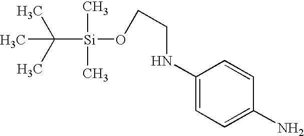

Under argon, 4 g of palladium on activated carbon (10%) are added to a solution of 59.5 g (201 mmol) of N-(2-{[tert-butyl(dimethyl)silyl]oxy}ethyl)-4-nitroaniline in 500 ml of ethanol, and the mixture is hydrogenated in an atmosphere of hydrogen at RT and under atmospheric pressure. The catalyst is separated off over a filter layer and washed with ethanol, and the filtrate is concentrated under reduced pressure.

Yield: 53 g (quant.)

LC-MS (Method 2): Rₜ=1.83 min;

MS (ESIpos): m/z=267 [M+H]⁺;

¹H-NMR (300 MHz, DMSO-d₆): δ=6.42-6.30 (m, 4H), 4.48 (t, 1H), 4.21 (br. s, 2H), 3.68-3.58 (m, 2H), 3.04-2.93 (m, 2H), 0.82 (s, 9H), 0.0 (s, 6H).

### Example 4A

### N-(3-{[tert-Butyl(dimethyl)silyl]oxy}propyl)benzene-1,4-diamine

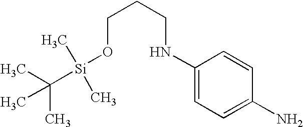

The title compound is prepared by a reaction sequence analogous to the sequence described in Example 3A.

LC-MS (Method 6): Rₜ=1.73 min;

MS (ESIpos): m/z=281 [M+H]⁺;

¹H-NMR (400 MHz, DMSO-d₆): δ=6.39 (d, 2H), 6.30 (d, 2H), 4.56 (br. s, 1H), 4.19 (br. s, 2H), 3.69-3.60 (m, 2H), 2.97-2.88 (m, 2H), 1.70-1.60 (m, 2H), 0.83 (s, 9H), 0.0 (s, 6H).

### Example 5A

### Methyl 2-{[(5-chloro-2-thienyl)carbonyl]amino}benzoate

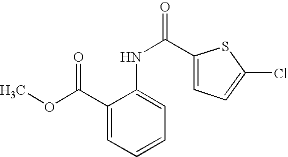

According to General Method II, 723 mg (4.78 mmol) of methyl anthranilate are reacted with 1.30 g (7.17 mmol, 1.5 eq.) of 5-chlorothiophene-2-carbonyl chloride. Trituration of the crude products with acetonitrile gives the title compound.

Yield: 650 mg (46% of theory)

LC-MS (Method 1): Rₜ=2.76 min;

MS (ESIpos): m/z=296 [M+H]⁺;

¹H-NMR (400 MHz, DMSO-d₆): δ=11.40 (s, 1H), 8.29 (d, 1H), 7.98 (d, 1H), 7.70 (d, 1H), 7.67 (t, 1H), 7.32 (d, 1H), 7.27 (t, 1H), 3.89 (s, 3H).

### Example 6A

### Methyl 2-{[(5-chloro-2-thienyl)carbonyl]amino}-4-methylbenzoate

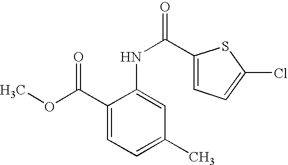

According to General Method II, 1.83 g (11.05 mmol) of methyl 2-amino-4-methylbenzoate [for the preparation, see G. Mayer, Chem. Ber. 1930, 63, 1455] are reacted with 3.00 g (16.57 mmol, 1.5 eq.) of 5-chlorothiophene-2-carbonyl chloride. Trituration of the crude products with acetonitrile gives the title compound.

Yield: 2.86 g (83% of theory)

HPLC (Method 7): Rₜ=5.41 min;

MS (DCI, NH₃): m/z=327 [M+NH₄]⁺;

¹H-NMR (400 MHz, DMSO-d₆): δ=11.49 (s, 1H), 8.20 (s, 1H), 7.90 (d, 1H), 7.69 (d, 1H), 7.32 (d, 1H), 7.09 (d, 1H), 3.88 (s, 3H), 2.39 (s, 3H).

### Example 7A

### Methyl 2-{[(5-chloro-2-thienyl)carbonyl]amino}-4-(trifluoromethyl)benzoate

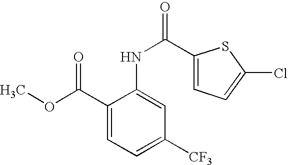

According to General Method II, 1.79 g (8.17 mmol) of methyl 2-amino-4-(trifluoromethyl)-benzoate [for the preparation, see F. T. Hill et al., J. Med. Chem. 1983, 26, 865-869] are reacted with 2.22 g (12.25 mmol, 1.5 eq.) of 5-chlorothiophene-2-carbonyl chloride. Trituration of the crude product with acetonitrile gives the title compound.

Yield: 1.95 g (66% of theory)

HPLC (Method 7): Rₜ=5.52 min;

MS (DCI, NH₃): m/z=381 [M+NH₄]⁺;

¹H-NMR (400 MHz, DMSO-d₆): δ=11.39 (s, 1H), 8.58 (s, 1H), 8.15 (d, 1H), 7.75 (d, 1H), 7.64 (d, 1H), 7.35 (d, 1H), 3.90 (s, 3H).

### Example 8A

### Methyl 4-chloro-2-{[(5-chloro-2-thienyl)carbonyl]amino}benzoate

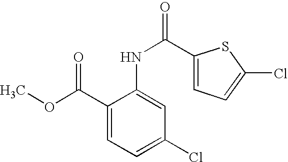

According to General Method II, 800 mg (4.31 mmol) of methyl 2-amino-4-chlorobenzoate are reacted with 1.17 g (6.47 mmol, 1.5 eq.) of 5-chlorothiophene-2-carbonyl chloride. The title compound is isolated by precipitation by adding water to the reaction mixture.

Yield: 1.37 g (96% of theory)

LC-MS (Method 3): Rₜ=3.24 min;

MS (ESIpos): m/z=330 [M+H]⁺.

### Example 9A

### Methyl 4-chloro-2-[(4-chlorobenzoyl)amino]benzoate

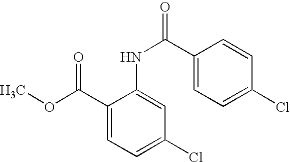

According to General Method II, 2.07 g (11.13 mmol) of methyl 2-amino-4-chlorobenzoate are reacted with 2.92 g (16.69 mmol, 1.5 eq.) of 4-chlorobenzoyl chloride. The title compound is isolated by flash chromatography on silica gel (mobile phase: dichloromethane/methanol 200:1).

Yield: 2.71 g (75% of theory)

LC-MS (Method 1): Rₜ=2.99 min;

MS (ESIpos): m/z=324 [M+H]⁺.

### Example 10A

### Methyl 2-{[(5-chloro-2-thienyl)carbonyl]amino}-4-fluorobenzoate

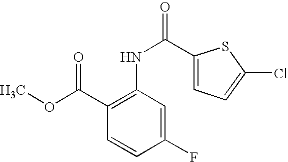

According to General Method II, 300 mg (1.46 mmol) of methyl 2-amino-4-fluorobenzoate [A. Cagir et al., Bioorg. Med. Chem. Lett. 2004, 14, 2051-2054] are reacted with 396 mg (2.19 mmol, 1.5 eq.) of 5-chlorothiophene-2-carbonyl chloride. The title compound is isolated by flash chromatography on silica gel (mobile phase: dichloromethane/methanol 98:2).

Yield: 307 mg (89% purity, 60% of theory)

LC-MS (Method 1): Rₜ=2.88 min;

MS (ESIpos): m/z=314 [M+H]⁺.

### Example 11A

### Methyl 4-methoxy-2-{[(5-chloro-2-thienyl)carbonyl]amino}benzoate

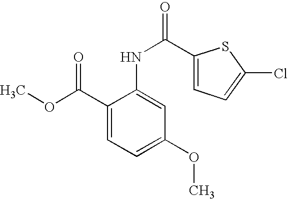

### Step a): Methyl 2-amino-4-methoxybenzoate

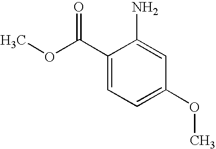

1000 mg (6.0 mmol) of 2-amino-4-methoxybenzoic acid are reacted according to General Method I.

Yield: 400 mg (37% of theory)

LC-MS (Method 3): Rₜ=1.96 min;

MS (ESIpos): m/z=182 [M+H]⁺.

### Step b): Methyl 4-methoxy-2-{[(5-chloro-2-thienyl)carbonyl]amino}benzoate

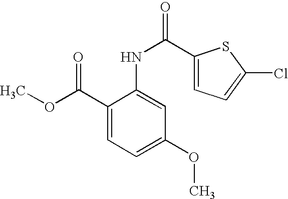

According to General Method II, 650 mg (3.6 mmol) of methyl 2-amino-4-methoxybenzoate are reacted with 974 mg (5.4 mmol, 1.5 eq.) of 5-chlorothiophene-2-carbonyl chloride. The title compound is purified by column chromatography on silica gel (mobile phase: cyclohexane/ethyl acetate 4:1).

Yield: 377 mg (31% of theory)

HPLC (Method 7): Rₜ=5.30 min;

MS (ESIpos): m/z=326 [M+H]⁺.

### Example 12A

### Methyl 2-{[(5-chloro-2-thienyl)carbonyl]amino}-4-cyanobenzoate

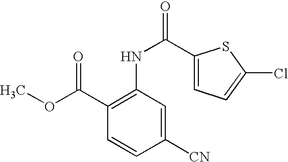

### Step a): 2-Amino-4-cyanobenzoic acid

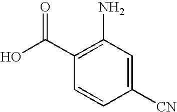

20 mg of palladium (5% on activated carbon) are added to a solution of 4000 mg (20.82 mmol) of 4-cyano-2-nitrobenzoic acid [Chan, R. L., Bruice, T. C., J. Am. Chem. Soc. 1977, 99, 6721-6730] in 50 ml of ethanol. After dropwise addition of 2.5 ml of hydrazine monohydrate, the reaction mixture is stirred under reflux for 18 h. The catalyst is filtered off over kieselguhr and the filtrate is concentrated under reduced pressure. The residue is dissolved in 20 ml 5% strength sodium carbonate solution and adjusted to pH 5 with 1 N hydrochloric acid. The resulting precipitate is separated off, washed with water and dried under reduced pressure.

Yield: 2220 mg (80% purity, 53% of theory)

LC-MS (Method 1): Rₜ=1.19 min;

MS (ESIpos): m/z=163 [M+H]⁺.

### Step b):Methyl 2-amino-4-cyanobenzoate

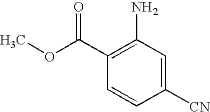

2000 mg (9.87 mmol) of 2-amino-4-cyanobenzoic acid are reacted according to General Method I.

Yield: 750 mg (84% purity, 43% of theory)

LC-MS (Method 5): Rₜ=3.38 min;

MS (ESIpos): m/z=177 [M+H]⁺.

### Step c): Methyl 2-{[(5-chloro-2-thienyl)carbonyl]amino}-4-cyanobenzoate

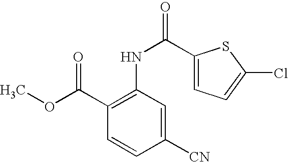

According to General Method II, 818 mg (4.64 mmol) of methyl 2-amino-4-cyanobenzoate are reacted with 1261 mg (6.97 mmol, 1.5 eq.) of 5-chlorothiophene-2-carbonyl chloride. The title compound precipitates from the reaction mixture.

Yield: 970 mg (85% purity, 55% of theory)

LC-MS (Method 3): Rₜ=2.87 min;

MS (ESIpos): m/z=321 [M+H]⁺.

### Example 13A

### 3-{[(5-Chloro-2-thienyl)carbonyl]amino}pyridine-2-carboxylic acid

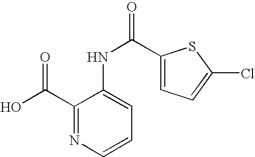

### Step a): Methyl 3-aminopyridine-2-carboxylate

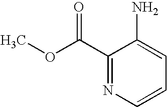

300 mg (6.0 mmol) of 3-aminopyridine-2-carboxylic acid are reacted according to General Method I.

Yield: 154 mg (45% of theory)

LC-MS (Method 5): Rₜ=1.62 min;

MS (ESIpos): m/z=153 [M+H]⁺.

### Step b): Methyl 3-{[(5-chloro-2-thienyl)carbonyl]amino}pyridine-2-carboxylate

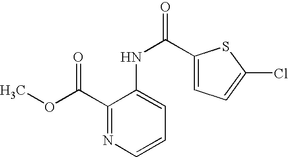

According to General Method II, 150 mg (1.0 mmol) of methyl 2-aminopyridine-3-carboxylate are reacted with 268 mg (1.5 mmol, 1.5 eq.) of 5-chlorothiophene-2-carbonyl chloride. The crude product is taken up in dichloromethane and washed with 25 ml of 0.5 N aqueous sodium hydroxide solution. The organic phase is dried over sodium sulfate, filtered and concentrated.

Yield: 233 mg (80% of theory)

LC-MS (Method 2): Rₜ=2.37 min;

MS (ESIpos): m/z=297 [M+H]⁺.

### Step c): 3-{[(5-Chloro-2-thienyl)carbonyl]amino}pyridine-2-carboxylic acid

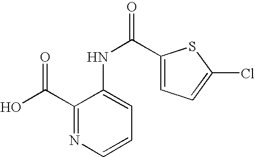

According to General Method III, 212 mg (0.71 mmol) of methyl 3-{[(5-chloro-2-thienyl)-carbonyl]amino}pyridine-2-carboxylate are reacted with 90 mg of lithium hydroxide monohydrate (2.14 mmol, 3 eq.).

Yield: 161 mg (77% of theory)

LC-MS (Method 3): Rₜ=1.62 min;

MS (ESIpos): m/z=283 [M+H]⁺.

### Example 14A

### 4-{[(5-Chloro-2-thienyl)carbonyl]amino}pyridine-3-carboxylic acid

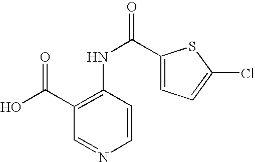

### Step a): Methyl 4-aminopyridine-3-carboxylate

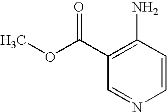

A little at a time, 4400 mg (31.86 mmol) of 4-aminopyridine-3-carboxylic acid are added to 12 ml of ice-cooled concentrated sulfuric acid. 70 ml of methanol are then added slowly. The reaction mixture is stirred under reflux (oil bath temperature 75° C.) for 20 h. The reaction solution is poured onto about 120 g of ice and neutralized with sodium carbonate. After extraction with dichloromethane, the organic phase is dried over magnesium sulfate and filtered. After 24 h of standing, the title compound crystallizes from the filtrate.

Yield: 3310 mg (67% of theory)

LC-MS (Method 9): Rₜ=1.59 min;

MS (ESIpos): m/z=153 [M+H]⁺.

### Step b): Methyl 4-{[(5-chloro-2-thienyl)carbonyl]amino}pyridine-3-carboxylate

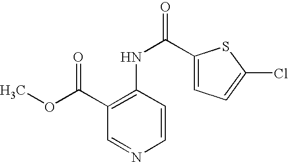

According to General Method II, 2000 mg (13.15 mmol) of methyl 4-aminopyridine-3-carboxylate are reacted with 3570 mg (19.72 mmol, 1.5 eq.) of 5-chlorothiophene-2-carbonyl chloride. After trituration with diethyl ether, the title compound crystallizes from the concentrated filtrate.

Yield: 1370 mg (35% of theory)

LC-MS (Method 2): Rₜ=2.45 min;

MS (ESIpos): m/z=297 [M+H]⁺.

### Step c): 4-{[(5-Chloro-2-thienyl)carbonyl]amino}pyridine-3-carboxylic acid

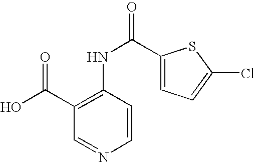

According to General Method III, 2.04 g (6.88 mmol) of methyl 4-{[(5-chloro-2-thienyl)carbonyl]-amino}pyridine-3-carboxylate are reacted with 0.87 g of lithium hydroxide monohydrate (20.63 mmol, 3 eq.).

Yield: 1.94 g (99% of theory)

LC-MS (Method 1): Rₜ=1.30 min;

MS (ESIpos): m/z=283 [M+H]⁺.

### Example 15A

### 3-{[(5-Chloro-2-thienyl)carbonyl]amino}pyridine-4-carboxylic acid

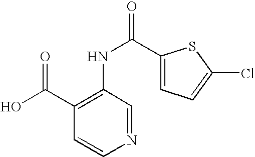

### Step a): Methyl 3-aminopyridine-4-carboxylate

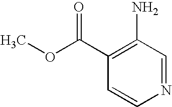

1030 mg (7.46 mmol) of 3-aminopyridine-4-carboxylic acid are reacted according to General Method I.

Yield: 1040 mg (89% of theory)

LC-MS (Method 5): Rₜ=1.90 min;

MS (ESIpos): m/z=153 [M+H]⁺.

### Step b): Methyl 3-{[(5-chloro-2-thienyl)carbonyl]amino}pyridine-4-carboxylate

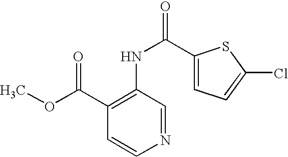

According to General Method II, 3.00 g (19.72 mmol) of methyl 3-aminopyridine-4-carboxylate are reacted with 5.35 g (29.58 mmol, 1.5 eq.) of 5-chlorothiophene-2-carbonyl chloride.

Yield: 5.91 g (98% of theory)

LC-MS (Method 2): Rₜ=2.52 min;

MS (ESIpos): m/z=297 [M+H]⁺.

### Step c): 3-{[(5-Chloro-2-thienyl)carbonyl]amino}pyridine-4-carboxylic acid

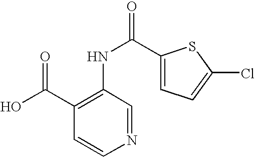

According to General Method III, 6.0 g (20 mmol) of methyl 3-{[(5-chloro-2-thienyl)carbonyl]-amino}pyridine-4-carboxylate are reacted with 2.5 g of lithium hydroxide monohydrate (60 mmol, 3 eq.).

Yield: 4.4 g (74% of theory)

LC-MS (Method 3): Rₜ=1.62 min;

MS (ESIpos): m/z=283 [M+H]⁺.

### Example 16A

### Methyl 3-[(4-chlorobenzoyl)amino]isonicotinate

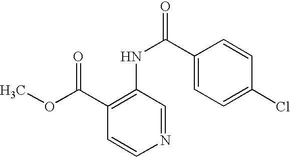

According to General Method II, 445 mg (2.93 mmol) of methyl 3-aminoisonicotinate [for the preparation, see S. L. Gwaltney et al., Bioorg. Med. Chem. Lett. 2003, 13, 1359-1362] are reacted with 768 mg (4.39 mmol, 1.5 eq.) of 4-chlorobenzoyl chloride. The title compound is isolated by flash chromatography on silica gel (mobile phase: dichloromethane).

Yield: 365 mg (43% of theory)

LC-MS (Method 1): Rₜ=2.09 min;

MS (ESIpos): m/z=291 [M+H]⁺.

### Example 17A

### 2-{[(5-Chloro-2-thienyl)carbonyl]amino}nicotinic acid

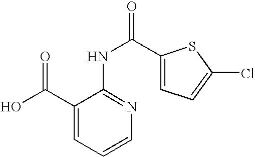

### Step a): Methyl 2-{[(5-chloro-2-thienyl)carbonyl]amino}nicotinate

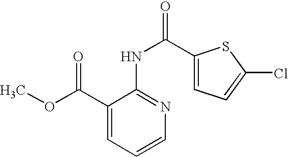

According to General Method II, 1000 mg (6.6 mmol) of methyl 2-aminonicotinate are reacted with 1785 mg (9.9 mmol, 1.5 eq.) of 5-chlorothiophene-2-carbonyl chloride. The crude product is taken up in dichloromethane and washed with 25 ml of 0.5 N aqueous sodium hydroxide solution. The organic phase is dried over sodium sulfate, filtered and concentrated. The residue is taken up in 10 ml of acetonitrile, and the solid that remains is filtered off. The solid is then washed with diethyl ether and dried under high vacuum.

Yield: 630 mg (32% of theory)

LC-MS (Method 1): Rₜ=2.97 min;

MS (ESIpos): m/z=297 [M+H]⁺.

### Step b): 2-{[(5-Chloro-2-thienyl)carbonyl]amino}nicotinic acid

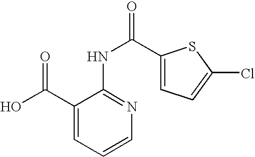

According to General Method III, 352 mg (1.2 mmol) of methyl 2-{[(5-chloro-2-thienyl)carbonyl]-amino}nicotinate are reacted with 149 mg of lithium hydroxide monohydrate (3.6 mmol, 3 eq.).

Yield: 320 mg (90% purity, 86% of theory)

LC-MS (Method 3): Rₜ=1.38 min;

MS (ESIpos): m/z=283 [M+H]⁺.

### Example 18A

### 3-[(4-Chlorobenzoyl)amino]pyrazine-2-carboxylic acid

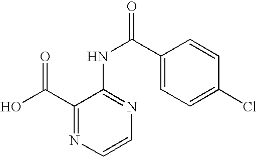

### Step a): Methyl 3-[(4-chlorobenzoyl)amino]pyrazine-2-carboxylate

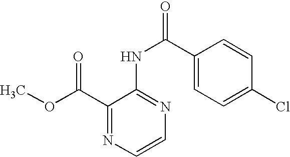

2.0 g (13.0 mmol) of methyl 3-aminopyrazine-2-carboxylate and 11.4 g (65.3 mmol, 5.0 eq.) of 4-chlorobenzoyl chloride in 20 ml of acetonitrile are heated under reflux. After 24 hours, the mixture is cooled to room temperature, and 25 ml of diethyl ether are added to the suspension. The solid is separated off. The title compound precipitates from the filtrate and is isolated by filtration.

Yield: 2.9 g (71% of theory)

LC-MS (Method 1): Rₜ=1.69 min;

MS (ESIpos): m/z=292 [M+H]⁺.

### Step b): 3-[(4-Chlorobenzoyl)amino]pyrazine-2-carboxylic acid

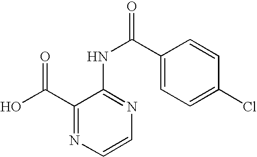

According to General Method III, 583 mg (2.0 mmol) of methyl 3-[(4-chlorobenzoyl)amino]-pyrazine-2-carboxylate are reacted with 252 mg of lithium hydroxide monohydrate (6.0 mmol, 3 eq.).

Yield: 61 mg (11% of theory)

LC-MS (Method 1): Rₜ=1.18 min;

MS (ESIpos): m/z=278 [M+H]⁺.

### Example 19A

### Methyl 4-{[(5-chloro-2-thienyl)carbonyl]amino}-2-methylpyrimidine-5-carboxylate

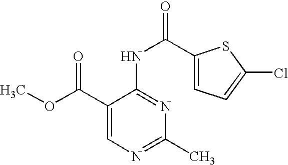

According to General Method II, 1500 mg (9.0 mmol) of methyl 4-amino-2-methylpyrimidine-5-carboxylate are reacted with 2437 mg (13.5 mmol, 1.5 eq.) of 5-chlorothiophene-2-carbonyl chloride. The crude product is taken up in dichloromethane and washed twice with 50 ml of 0.5 N aqueous sodium hydroxide solution. The organic phase is dried over sodium sulfate, filtered and concentrated. The crude product is purified by column chromatography on silica gel (mobile phase: dichloromethane/methanol 95:5).

Yield: 294 mg (10% of theory)

LC-MS (Method 2): Rₜ=2.23 min;

MS (ESIpos): m/z=312 [M+H]⁺.

### Example 20A

### 4-{[(5-Chloro-2-thienyl)carbonyl]amino}thiophene-3-carboxylic acid

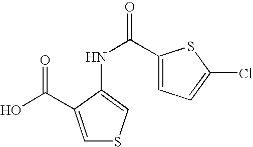

### Step a): Methyl 4-{[(5-chloro-2-thienyl)carbonyl]amino}thiophene-3-carboxylate

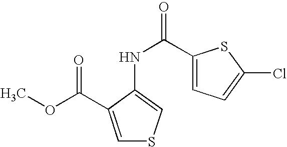

According to General Method II, 1000 mg (5.2 mmol) of methyl 3-aminothiophene-2-carboxylate are reacted with 1402 mg (7.7 mmol, 1.5 eq.) of 5-chlorothiophene-2-carbonyl chloride. The crude product is taken up in dichloromethane and washed twice with 50 ml of 0.5 N aqueous sodium hydroxide solution. The organic phase is dried over sodium sulfate, filtered and concentrated.

Yield: 1770 mg (74% purity, 86% of theory)

LC-MS (Method 2): Rₜ=2.48 min;

MS (ESIpos): m/z=288 [M+H]⁺.

### Step b): 4-{[(5-Chloro-2-thienyl)carbonyl]amino}thiophene-3-carboxylic acid

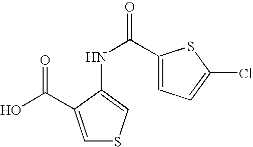

According to General Method III, 1539 mg (5.1 mmol) of methyl 4-{[(5-chloro-2-thienyl)-carbonyl]amino}thiophene-3-carboxylate are reacted with 642 mg of lithium hydroxide monohydrate (15.3 mmol, 3 eq.).

Yield: 1350 mg (88% of theory)

LC-MS (Method 3): Rₜ=2.84 min;

MS (ESIpos): m/z=302 [M+H]⁺.

### Example 21A

### 4-{[(5-Chloro-2-thienyl)carbonyl]amino}isothiazole-3-carboxylic acid

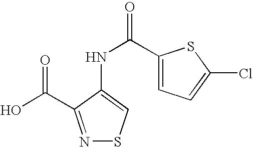

### Step a): Methyl 4-aminoisothiazole-3-carboxylate

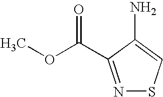

1000 mg (5.5 mmol) of 4-aminoisothiazole-3-carboxylic acid are reacted according to General Method I.

Yield: 690 mg (79% of theory)

LC-MS (Method 5): Rₜ=2.11 min;

MS (ESIpos): m/z=159 [M+H]⁺.

### Step b): Methyl 4-{[(5-chloro-2-thienyl)carbonyl]amino}isothiazole-3-carboxylate

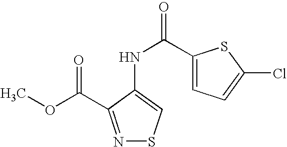

According to General Method II, 680 mg (4.3 mmol) of methyl 4-aminoisothiazole-3-carboxylate are reacted with 1167 mg (6.4 mmol, 1.5 eq.) of 5-chlorothiophene-2-carbonyl chloride. The residue that remains is taken up in dichloromethane and washed with 25 ml of 0.5 N aqueous sodium hydroxide solution. The organic phase is dried over sodium sulfate, filtered and concentrated. The crude product is used without purification for the next step.

LC-MS (Method 3): Rₜ=2.50 min;

MS (ESIpos): m/z=303 [M+H]⁺.

### Step c): 4-{[(5-Chloro-2-thienyl)carbonyl]amino}isothiazole-3-carboxylic acid

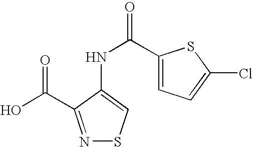

According to General Method III, 1150 g (3.8 mmol) of methyl 4-{[(5-chloro-2-thienyl)carbonyl]-amino}isothiazole-3-carboxylate are reacted with 478 mg of lithium hydroxide monohydrate (11.4 mmol, 3 eq.).

Yield: 1020 mg (93% of theory)

LC-MS (Method 2): Rₜ=2.19 min;

MS (ESIpos): m/z=289 [M+H]⁺.

### Example 22A

### Ethyl 3-{[(5-chloro-2-thienyl)carbonyl]amino}-1-methyl-1H-pyrazole-4-carboxylate

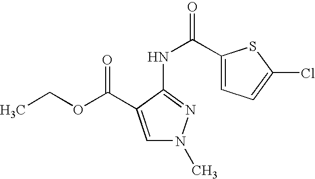

According to General Method II, 554 mg (3.28 mmol) of ethyl 3-amino-1-methyl-1H-pyrazole-4-carboxylate [K. Morimoto et al., J. Heterocycl. Chem. 1997, 34, 537-540] are reacted with 889 mg (4.91 mmol, 1.5 eq.) of 5-chlorothiophene-2-carbonyl chloride. The title compound is isolated by flash chromatography on silica gel (mobile phase: dichloromethane/methanol 100:1).

Yield: 623 mg (61% of theory)

LC-MS (Method 2): Rₜ=2.31 min;

MS (ESIpos): m/z=314 [M+H]⁺;

¹H-NMR (400 MHz, DMSO-d₆): δ=10.35 (s, 1H), 8.28 (s, 1H), 7.81 (d, 1H), 7.36 (d, 1H), 4.10 (qd, 2H), 3.84 (s, 3H), 1.13 (t, 3H).

### Example 23A

### 3-{[(5-Chloro-2-thienyl)carbonyl]amino}thiophene-2-carboxylic acid

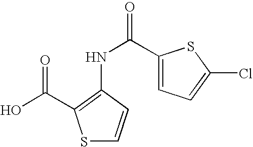

### Step a): Methyl 3-{[(5-chloro-2-thienyl)carbonyl]amino}thiophene-2-carboxylate

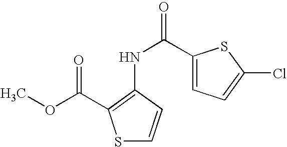

According to General Method II, 500 mg (3.18 mmol) of methyl 3-aminothiophene-2-carboxylate are reacted with 864 mg (4.77 mmol, 1.5 eq.) of 5-chlorothiophene-2-carbonyl chloride. Trituration of the crude product with acetonitrile gives the title compound.

Yield: 818 mg (74% of theory)

LC-MS (Method 3): Rₜ=2.89 min;

MS (ESIpos): m/z=302 [M+H]⁺.

### Step b): 3-{[(5-Chloro-2-thienyl)carbonyl]amino}thiophene-2-carboxylic acid

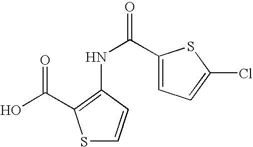

A solution of 412 mg (1.37 mmol) of methyl 3-{[(5-chloro-2-thienyl)carbonyl]amino}thiophene-2-carboxylate and 65.4 mg (2.73 mmol, 2 eq.) of lithium hydroxide in 20 ml of THF/water (3:1) is stirred at RT for 4 h. The reaction mixture is freed from the THF and adjusted to pH 1 using 1 N hydrochloric acid. The resulting precipitate is filtered off, washed with water and dried under reduced pressure.

Yield: 368 mg (94% of theory)

LC-MS (Method 2): Rₜ=2.47 min;

MS (ESIpos): m/z=288 [M+H]⁺.

### Example 24A

### Methyl 2-{[(5-chloro-2-thienyl)carbonyl]amino}-5-(dimethylamino)-4-methylbenzoate

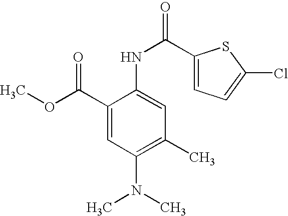

### Step a): Methyl 2-amino-4-methyl-5-nitrobenzoate

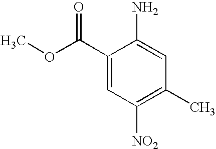

5.7 g (29.1 mmol) of 2-amino-4-methyl-5-nitrobenzoic acid are reacted according to General Method I.

Yield: 5.3 g (91% purity, 79% of theory)

LC-MS (Method 2): Rₜ=2.29 min;

MS (ESIpos): m/z=211 [M+H]⁺.

### Step b): Methyl 2-{[(5-chloro-2-thienyl)carbonyl]amino}-4-methyl-5-nitrobenzoate

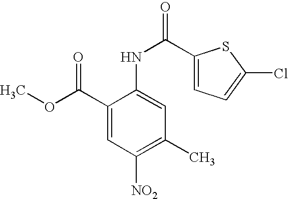

According to General Method II, 5.3 g (22.7 mmol) of methyl 2-amino-4-methyl-5-nitrobenzoate are reacted with 6.2 g (34.0 mmol, 1.5 eq.) of 5-chlorothiophene-2-carbonyl chloride. Water is added to the reaction mixture. The resulting precipitate is filtered off, stirred with acetonitrile, filtered again, washed with cyclohexane and dried under reduced pressure.

Yield: 7.8 g (76% purity, 74% of theory)

LC-MS (Method 1): Rₜ=2.94 min;

MS (ESIpos): m/z=355 [M+H]⁺.

### Step c): Methyl 5-amino-2-{[(5-chloro-2-thienyl)carbonyl]amino)}-4-methylbenzoate

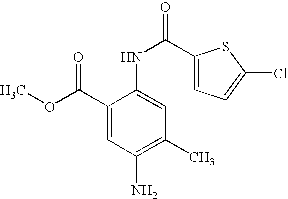

At room temperature, a catalytic amount of Raney nickel is added to a solution of 100 mg (0.28 mmol) of methyl 2-{[(5-chloro-2-thienyl)carbonyl]amino}-4-methyl-5-nitrobenzoate in 2 ml of THF. With vigorous stirring, 21 μl (0.42 mmol, 1.5 eq.) of hydrazine hydrate are added dropwise, and two more catalytic amounts of Raney nickel are then added with TLC monitoring until the reaction has gone to completion. Sodium sulfate is added to the reaction mixture, the mixture is filtered over kieselguhr and the kieselguhr is washed thoroughly with dichloromethane. The combined filtrates are concentrated under reduced pressure.

Yield: 45 mg (73% purity, 36% of theory)

LC-MS (Method 3): Rₜ=2.53 min;

MS (ESIpos): m/z=325 [M+H]⁺;

¹H-NMR (400 MHz, DMSO-d₆): δ=10.88 (s, 1H), 7.78 (s, 1H), 7.63 (s, 1H), 7.33-7.19 (m, 2H), 5.11 (s, 2H), 3.81 (s, 3H), 2.12 (s, 3H).

### Step d): Methyl 2-{[(5-chloro-2-thienyl)carbonyl]amino}-5-(dimethylamino)-4-methylbenzoate

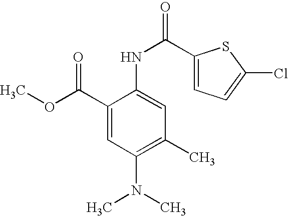

At room temperature, 231 mg (6.10 mmol, 6 eq.) of sodium borohydride are added to a solution of 330 mg (1.02 mmol) of methyl 5-amino-2-{[(5-chloro-2-thienyl)carbonyl]amino}-4-methyl-benzoate in 8 ml of THF. With vigorous stirring, this mixture is added dropwise to a mixture of 322 μl (4.06 mmol, 4 eq.) of a 35% strength formaldehyde solution and 0.7 eq. of 3 N sulfuric acid in 8 ml of THF, and after half is added, a further 0.7 eq. of 3 N sulfuric acid are added dropwise in parallel. 5 min after the addition has finished, the reaction mixture is made alkaline with 1.4 ml of 1 N aqueous sodium hydroxide solution, and water and ethyl acetate are added. After phase separation, the organic phase is dried over sodium sulfate, filtered and concentrated under reduced pressure.

Yield: 341 mg (92% purity, 88% of theory)

LC-MS (Method 1): Rₜ=2.77 min;

MS (ESIpos): m/z=353 [M+H]⁺;

¹H-NMR (400 MHz, DMSO-d₆): δ=11.19 (s, 1H), 8.07 (s, 1H), 7.67 (d, 1H), 7.56 (s, 1H), 7.31 (d, 1H), 3.86 (s, 3H), 2.64 (s, 6H), 2.33 (s, 3H).

### Example 25A

### Methyl 2-{[(5-chloro-2-thienyl)carbonyl]amino}-5-fluoro-4-methylbenzoate

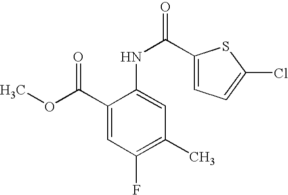

### Step a) Methyl 2-amino-5-fluoro-4-methylbenzoate

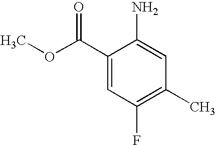

1.28 g (7.57 mmol) of 2-amino-5-fluoro-4-methylbenzoic acid are reacted according to General Method I.

Yield: 1.04 g (96% purity, 72% of theory)

LC-MS (Method 2): Rₜ=2.29 min;

MS (ESIpos): m/z=184 [M+H]⁺;

¹H-NMR (400 MHz, DMSO-d₆): δ=7.32 (d, 1H), 6.65 (d, 1H), 6.47 (s, 2H), 3.77 (s, 3H), 2.16 (s, 3H).

### Step b): Methyl 2-{[(5-chloro-2-thienyl)carbonyl]amino}-5-fluoro-4-methylbenzoate

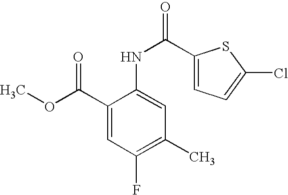

According to General Method II, 500 mg (2.73 mmol) of methyl 2-amino-5-fluoro-4-methyl-benzoate are reacted with 741 mg (4.09 mmol, 1.5 eq.) of 5-chlorothiophene-2-carbonyl chloride.

The reaction mixture is concentrated under reduced pressure and the title compound is isolated by trituration of the crude product with acetonitrile.

Yield: 560 mg (63% of theory)

LC-MS (Method 2): Rₜ=3.32 min;

MS (ESIpos): m/z=328 [M+H]⁺;

¹H-NMR (400 MHz, DMSO-d₆): δ=11.15 (s, 1H), 8.13 (d, 1H), 7.70 (d, 1H), 7.65 (d, 1H), 7.32 (d, 1H), 3.86 (s, 3H), 2.32 (s, 3H).

### Example 26A

### Ethyl 3-{[(5-chloropyridin-2-yl)carbonyl]amino}-1-methyl-1H-pyrazole-4-carboxylate

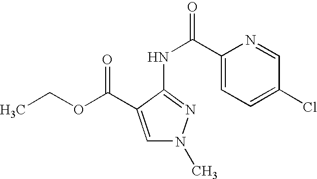

According to General Method II, 63 mg (0.37 mmol) of ethyl 3-amino-1-methyl-1H-pyrazole-4-carboxylate [K. Morimoto et al., J. Heterocycl. Chem. 1997, 34, 537-540] are reacted with 98 mg (0.56 mmol, 1.5 eq.) of 5-chloropyridine-2-carbonyl chloride. The title compound is isolated by flash chromatography on silica gel (mobile phase: dichloromethane/methanol 50:1).

Yield: 95 mg (83% of theory)

LC-MS (Method 1): Rₜ=1.87 min;

MS (ESIpos): m/z=309 [M+H]⁺;

¹H-NMR (400 MHz, DMSO-d₆): δ=11.15 (s, 1H), 8.80 (d, 1H), 8.30 (s, 1H), 8.21 (dd, 1H), 8.16 (d, 1H), 4.27 (qd, 2H), 3.85 (s, 3H), 1.30 (t, 3H).

### Example 27A

### Methyl 2{-[(5-chloro-2-thienyl)carbonyl]amino}-5-cyano-4-methylbenzoate

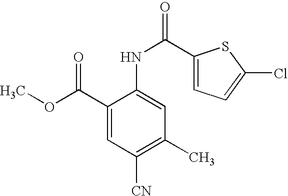

### Step a): Methyl 2-amino-4-methyl-5-nitrobenzoate

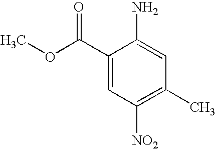

5.7 g (29.1 mmol) of 2-amino-4-methyl-5-nitrobenzoic acid [F. W. Lichtenthaler et al., Tetrahedron Lett. 1981, 22, 4397-4400] are initially charged in 250 ml of methanol, and 28.5 ml of concentrated sulfuric acid are added. The mixture is heated under reflux for 16 hours and then, after cooling to room temperature, poured onto ice. The pH is adjusted to 6 using solid sodium bicarbonate, the methanol is removed under reduced pressure and the precipitate is separated off. The solid is dried under high vacuum.

Yield: 5.3 g (87% of theory)

LC-MS (Method 2): Rₜ=2.29 min;

MS (ESIpos): m/z=211 [M+H]⁺.

### Step b): Methyl 2-{[(5-chloro-2-thienyl)carbonyl]amino}-4-methyl-5-nitrobenzoate

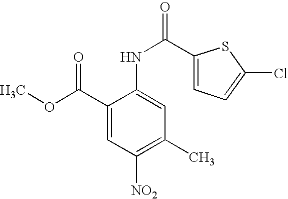

A solution of 5.3 g (22.7 mmol) of methyl 2-amino-5-cyano-4-methylbenzoate in 25 ml of THF is warmed to 55° C., and 3.7 ml (3.6 g, 2.0 eq.) of pyridine and 277 mg (2.3 mmol, 0.1 eq.) of 4-N,N-dimethylaminopyridine are added. 6.2 g (34.0 mmol, 1.5 eq.) of the compound of Example 1A, dissolved in 25 ml of THF, are then added dropwise. The mixture is heated under reflux for 18 hours. After cooling to room temperature, 250 ml of water are added. The precipitate is separated off, washed with water, triturated with acetonitrile, filtered again, washed with cyclohexane and dried under reduced pressure.

Yield: 7.8 g (97% of theory)

HPLC (Method 9): Rₜ=5.07 min;

MS (ESIneg): m/z=353 [M−H]⁻.

### Step c): Methyl 5-amino-2-{[(5-chloro-2-thienyl)carbonyl]amino}-4-methylbenzoate

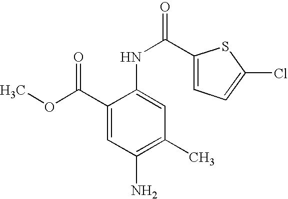

3.7 g (10.4 mmol) of methyl 2-{[(5-chloro-2-thienyl)carbonyl]amino}-4-methyl-5-nitrobenzoate are dissolved in 74 ml of THF, and a spatula tip of Raney nickel is added. With vigorous stirring, 0.8 ml (15.6 mmol, 1.5 eq.) of hydrazine hydrate is added dropwise. Three times, in each case after a reaction time of one hour, a further spatula tip of Raney nickel is added. After a total of four hours, sodium sulfate is added, the mixture is filtered over kieselguhr, the filter cake is washed with dichloromethane and the filtrate is concentrated under reduced pressure.

Yield: 3.3 g (96% of theory)

LC-MS (Method 1): Rₜ=2.32 min;

MS (ESIpos): m/z=325 [M+H]⁺;

¹H-NMR (400 MHz, DMSO-d₆): δ=10.89 (s, 1H), 7.79 (s, 1H), 7.63 (d, 1H), 7.27 (d, 1H), 7.24 (s, 1H), 5.11 (br. s, 2H), 3.81 (s, 3H), 2.13 (s, 3H).

### Step d): Methyl 2-{[(5-chloro-2-thienyl)carbonyl]amino}-5-cyano-4-methylbenzoate

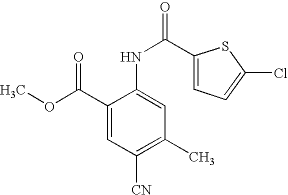

358 mg (4.0 mmol, 1.3 eq.) of copper(I) cyanide and 1.1 ml (952 mg, 3.0 eq.) of tert-butyl nitrite are initially charged in 3 ml of DMSO. At 50° C., 1.0 g (3.1 mmol) of methyl 5-amino-2-{[(5-chloro-2-thienyl)carbonyl]amino}-4-methylbenzoate is added a little at a time. The mixture is stirred at 60° C. for one hour, 6 ml of acetonitrile are then added and the solid residue is separated off. The filtrate is purified by RP-HPLC.

Yield: 58 mg (5% of theory)

LC-MS (Method 1): Rₜ=2.82 min;

MS (ESIneg): m/z=333 [M−H]⁻;

¹H-NMR (400 MHz, DMSO-d₆): δ=11.62 (s, 1H), 8.43 (s, 1H), 8.31 (s, 1H), 7.70 (d, 1H), 7.33 (d, 1H), 3.90 (s, 3H), 2.54 (s, 3H).

### Example 28A

### Methyl 4-{[(5-chloro-2-thienyl)carbonyl]amino}-2-(methylthio)-1,3-thiazole-5-carboxylate

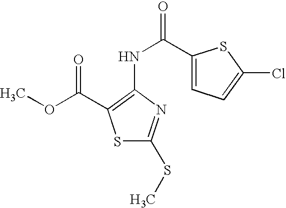

4.0 g (19.6 mmol) of methyl 4-amino-2-(methylthio)-1,3-thiazole-5-carboxylate are initially charged in 25 ml of THF and warmed to 55° C. 3.2 ml (3.1 g, 2.0 eq.) of pyridine and 239 mg (2.0 mmol, 0.1 eq.) of 4-N,N-dimethylaminopyridine are added to the solution. 5.3 g (29.4 mmol, 1.5 eq.) of the compound of Example 1A, dissolved in 25 ml of THF, are then added dropwise. The mixture is heated under reflux for 18 hours. After cooling to room temperature, 250 ml of water are added. The precipitate is separated off, washed with water, triturated with acetonitrile, filtered again, washed with cyclohexane and dried under reduced pressure.

Yield: 5.7 g (72% purity, 60% of theory)

LC-MS (Method 3): Rₜ=2.60 min;

MS (ESIpos): m/z=349 [M+H]⁺.

### Example 29A

### Methyl 2-{[(5-chloropyridin-2-yl)carbonyl]amino}-4-methyl benzoate

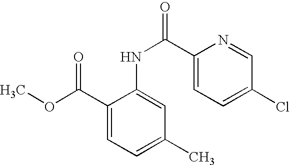

234 mg (1.3 mmol, 1.1 eq.) of the compound of Example 2A are added to a solution of 200 mg (1.2 mmol) of methyl 2-amino-4-methylbenzoate [G. Reissenweber et al., Angew. Chem. 1981, 93, 914-915] in 6 ml of dioxane and 0.25 ml of pyridine. The reaction mixture is stirred at room temperature for 12 h. After addition of water, the precipitate formed is filtered off, washed with diethyl ether and dried under reduced pressure.

Yield: 163 mg (44% of theory)

LC-MS (Method 3): Rₜ=2.90 min;

MS (ESIpos): m/z=305 [M+H]⁺.

### Working Examples

### General Method 1: Amide Coupling with Carboxylic Acids

A solution of the carboxylic acid in question and N,N-diisopropylethylamine (1.05 eq.) in dichloromethane (5 ml/mmol) is stirred at RT for 15 min, and a solution of the aniline derivative (1.0 eq.) in dichloromethane (5 ml/mmol) and O-(benzotriazol-1-yl)-N,N,N′-tetramethyluronium tetrafluoroborate (TBTU, 1.05 eq.) are then added. The reaction mixture is stirred at room temperature overnight. The reaction mixture is then washed with water, with saturated aqueous sodium bicarbonate solution and again with water. The organic phase is concentrated under reduced pressure, and ethyl acetate is added to the residue. The precipitated solid is filtered off and washed with pentane. The filtrate is concentrated under reduced pressure and the residue is purified by column chromatography on silica gel or by preparative RP-HPLC.

### General Method 2: Amide Coupling with Carboxylic Esters

Under argon and at 0° C., trimethylaluminum solution (2 M in hexane, 5 eq.) is added dropwise to a solution of the aniline (2 eq.) in dichloromethane (4 ml/mmol). The reaction mixture is allowed to warm to RT, stirred at RT for 15 min and again cooled to 0° C., and a solution of the carboxylic ester in question in dichloromethane (8 ml/mmol) is then added. The mixture is stirred further at RT, and a 20% strength potassium tartrate solution is then added dropwise (careful: vigorous foaming!). After addition of dichloromethane and phase separation, the organic phase is washed with water, dried over sodium sulfate, filtered and concentrated under reduced pressure. The crude product is purified by preparative RP-HPLC or by column chromatography on silica gel.

### General Method 3: Reaction with Cyanogen Bromide

Under argon, sodium bicarbonate (3 eq.) and cyanogen bromide solution (3 M in dichloromethane, about 1.2 eq.) are added to a solution of the coupling product in THF (10 ml/mmol), and the mixture is stirred at 40° C. After addition of water/dichloromethane and phase separation, the aqueous phase is reextracted with dichloromethane. The combined organic phases are washed with saturated aqueous sodium carbonate solution, dried over sodium sulfate, filtered and concentrated under reduced pressure. The particular purification of the crude product is described in the examples.

### General Method 4: Cyclization to the Iminooxazolidine Derivative in the Presence of Methanesulfonic Acid

At RT, 2.1-2.2 eq. of methanesulfonic acid are added to a solution of the N-cyano compound in acetonitrile (about 100 ml/mmol), and the mixture is stirred at RT until complete conversion of the starting material is achieved. The reaction mixture is then either concentrated and the crude product is purified by flash chromatography on silica gel or, if a precipitate has formed in the reaction mixture, this precipitate is filtered off, washed with acetonitrile and dried under high vacuum.

### Example 1

### 5-Chloro-N-[2-({[4-(2-imino-1,3-oxazolidin-3-yl)phenyl]amino}carbonyl)phenyl]thiophene-2-carboxamide methanesulfonate

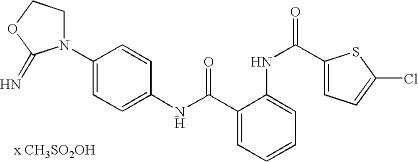

### Step a): N-{2-[({4-[(2-{[tert-Butyl(dimethyl)silyl]oxy}ethyl)amino]phenyl}amino)-carbonyl]phenyl}-5-chlorothiophene-2-carboxamide

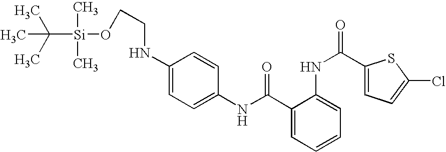

According to General Method 2, 638 mg (2.16 mmol) of the compound of Example 5A and 1.15 g (4.32 mmol, 2 eq.) of the compound of Example 3A are reacted in the presence of 5.4 ml of tri-methylaluminum solution (2 M in hexane, 10.8 mmol, 5 eq.). The title compound is isolated by flash chromatography on silica gel (mobile phase: dichloromethane/methanol 400:1).

Yield: 476 mg (40% of theory)

LC-MS (Method 1): Rₜ=3.34 min;

MS (ESIpos): m/z=530 [M+H]⁺;

¹H-NMR (400 MHz, DMSO-d₆): δ=12.07 (s, 1H), 10.20 (s, 1H), 8.31 (d, 1H), 7.89 (d, 1H), 7.57-7.49 (d, 1H and t, 1H), 7.33 (d, 2H), 7.24 (d, 1H), 7.21 (t, 1H), 6.55 (d, 2H), 5.42 (t, 1H), 3.68 (t, 22H), 3.10 (qd, 2H), 0.82 (s, 9H), 0.00 (s, 6H).

### Step b): N-{2-[({4-[(2-{[tert-Butyl(dimethyl)silyl]oxy}ethyl)(cyano)amino]phenyl}-amino)carbonyl]phenyl}-5-chlorothiophene-2-carboxamide

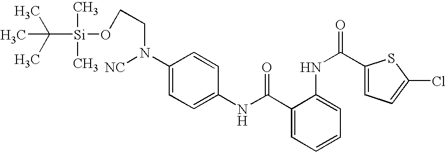

According to General Method 3, 458 mg (0.86 mmol) of N-{2-[({4-[(2-{[tert-butyl(dimethyl)-silyl]oxy}ethyl)amino]phenyl}amino)carbonyl]phenyl}-5-chlorothiophene-2-carboxamide are reacted with a total of 519 μl of cyanogen bromide solution (3 M in dichloromethane, 1.56 mmol, 1.8 eq.) in the presence of 218 mg (2.59 mmol, 3 eq.) of sodium bicarbonate. The title compound is isolated by flash chromatography on silica gel (mobile phase: dichloromethane/methanol 300:1).

Yield: 338 mg (87% purity, 61% of theory)

HPLC (Method 8): Rₜ=5.42 min;

MS (ESIpos): m/z=555 [M+H]⁺;

¹H-NMR (400 MHz, DMSO-d₆): δ=11.64 (s, 1H), 10.55 (s, 1H), 8.25 (d, 1H), 7.91 (d, 1H), 7.77 (d, 2H), 7.63 (d, 1H), 7.61 (t, 1H), 7.31 (t, 1H), 7.30 (d, 1H), 7.21 (d, 2H), 3.79-3.90 (m, 4H), 0.86 (s, 9H), 0.0 (s, 6H).

### Step c): 5-Chloro-N-[2-({[4-(2-imino-1,3-oxazolidin-3-yl)phenyl]amino}carbonyl)phenyl]-thiophene-2-carboxamide methanesulfonate

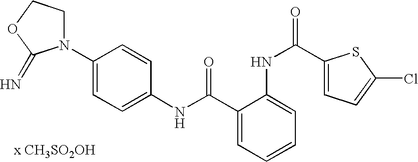

According to General Method 4, 216 mg (0.39 mmol) of N-{2-[({4-[(2-{[tert-butyl(dimethyl)silyl]-oxy}ethyl)(cyano)amino]phenyl}amino)carbonyl]phenyl}-5-chlorothiophene-2-carboxamide are reacted with 53 μl (0.82 mmol, 2.1 eq.) of methanesulfonic acid. The title compound is isolated by filtration of the precipitate formed, washing with acetonitrile and drying under reduced pressure.

Yield: 100 mg (48% of theory)

HPLC (Method 9): Rₜ=4.31 min;

MS (ESIpos): m/z=441 [M+H]⁺ (free base);

¹H-NMR (400 MHz, DMSO-d₆): δ=11.38 (s, 1H), 10.72 (s, 1H), 9.59 (br. s, 1H), 8.82 (br. s, 1H), 8.11 (d, 1H), 7.91-7.83 (2d, 3H), 7.68 (d, 1H), 7.62 (t, 1H), 7.51 (d, 2H), 7.35 (t, 1H), 7.30 (d, 1H), 4.87 (t, 2H), 4.24 (t, 2H), 2.30 (s, 3H).

### Example 2

### 5-Chloro-N-[2-({[4-(2-imino-1,3-oxazolidin-3-yl)phenyl]amino}carbonyl)-5-methylphenyl]-thiophene-2-carboxamide methanesulfonate

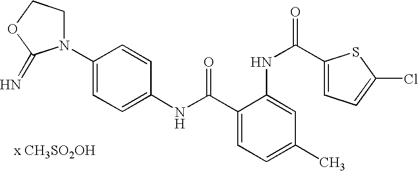

### Step a): N-{2-[({4-[(2-{[tert-Butyl(dimethyl)silyl]oxy}ethyl)amino]phenyl}amino)-carbonyl]-5-methylphenyl}-5-chlorothiophene-2-carboxamide

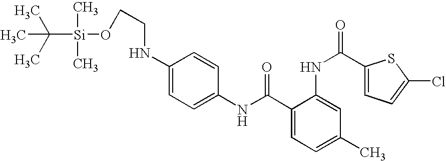

According to General Method 2, 800 mg (2.58 mmol) of the compound of Example 6A and 1.38 g (5.17 mmol, 2 eq.) of the compound of Example 3A are reacted in the presence of 6.5 ml of tri-methylaluminum solution (2 M in hexane, 12.9 mmol, 5 eq.). The title compound is isolated by flash chromatography on silica gel (mobile phase: dichloromethane/methanol 400:1).

Yield: 560 mg (40% of theory)

HPLC (Method 8): Rₜ=4.95 min;

MS (ESIpos): m/z=544 [M+H]⁺;

¹H-NMR (400 MHz, DMSO-d₆): δ=12.32 (s, 1H), 10.18 (s, 1H), 8.28 (s, 1H), 7.85 (d, 1H), 7.54 (d, 1H), 7.36 (d, 2H), 7.28 (d, 1H), 7.07 (d, 1H), 6.59 (d, 2H), 5.46 (t, 1H), 3.71 (t, 2H), 3.13 (qd, 2H), 2.38 (s, 3H), 0.87 (s, 9H), 0.03 (s, 6H).

### Step b): N-{2-[({4-[(2-{[tert-Butyl(dimethyl)silyl]oxy}ethyl)(cyano)amino]phenyl}-amino)carbonyl]-5-methylphenyl}-5-chlorothiophene-2-carboxamide

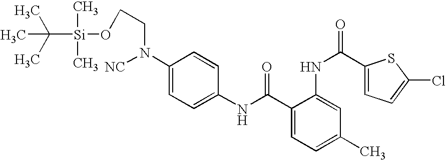

According to General Method 3, 560 mg (1.03 mmol) of N-{2-[({4-[(2-{[tert-butyl(dimethyl)-silyl]oxy}ethyl)amino]phenyl}amino)carbonyl]-5-methylphenyl}-5-chlorothiophene-2-carboxamide are reacted with a total of 481 μl of cyanogen bromide solution (3 M in dichloromethane, 1.44 mmol, 1.4 eq.) in the presence of 260 mg (3.09 mmol, 3 eq.) of sodium bicarbonate. The title compound is isolated by trituration of the crude product with diisopropyl ether.

Yield: 499 mg (85% of theory)

LC-MS (Method 3): Rₜ=3.52 min;

MS (ESIpos): m/z=569 [M+H]⁺;

¹H-NMR (400 MHz, DMSO-d₆): δ=11.89 (s, 1H), 10.49 (s, 1H), 8.19 (s, 1H), 7.87 (d, 1H), 7.75 (d, 2H), 7.61 (d, 1H), 7.29 (d, 1H), 7.22 (d, 2H), 7.13 (d, 1H), 3.89-3.79 (m, 4H), 2.40 (s, 3H), 0.85 (s, 9H), 0.0 (s, 6H).

### Step c): 5-Chloro-N-[2-({[4-(2-imino-1,3-oxazolidin-3-yl)phenyl]amino}carbonyl)-5-methylphenyl]thiophene-2-carboxamide methanesulfonate

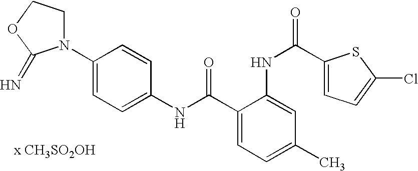

According to General Method 4, 499 mg (0.88 mmol) of N-{2-[({4-[(2-{[tert-butyl(di-methyl)silyl]oxy}ethyl)(cyano)amino]phenyl}amino)carbonyl]-5-methylphenyl}-5-chlorothiophene-2-carboxamide are reacted with 120 μl (1.84 mmol, 2.1 eq.) of methanesulfonic acid. The title compound is isolated by filtration of the precipitate formed, washing with acetonitrile and drying under reduced pressure.

Yield: 366 mg (74% of theory)

HPLC (Method 9): Rₜ=4.35 mm;

MS (ESIpos): m/z=455 [M+H]⁺ (free base);

¹H-NMR (400 MHz, DMSO-d₆): δ=11.62 (s, 1H), 10.68 (s, 1H), 9.60 (br. s, 1H), 8.84 (br. s, 1H), 8.08 (s, 1H), 7.89 (d, 2H), 7.85 (d, 1H), 7.64 (d, 1H), 7.53 (d, 2H), 7.31 (d, 1H), 7.17 (d, 1H), 4.89 (t, 2H), 4.25 (t, 2H), 2.41 (s, 3H), 2.32 (s, 3H).

### Example 3

### 5-Chloro-N-[2-({[4-(2-imino-1,3-oxazolidin-3-yl)phenyl]amino}carbonyl)-5-(trifluoromethyl)-phenyl]thiophene-2-carboxamide methanesulfonate

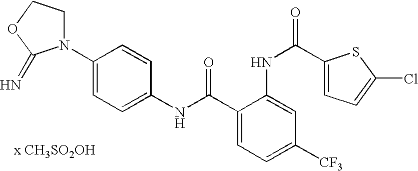

### Step a): N-[2-[({4-[(2-{[tert-Butyl(dimethyl)silyl]oxy}ethyl)amino]phenyl}amino)-carbonyl]-5-(trifluoromethyl)phenyl]-5-chlorothiophene-2-carboxamide

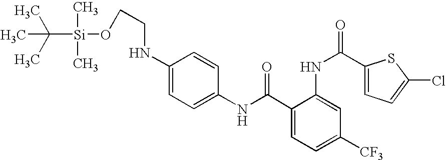

According to General Method 2, 800 mg (2.20 mmol) of the compound of Example 7A and 1.17 g (4.40 mmol, 2 eq.) of the compound of Example 3A are reacted in the presence of 5.5 ml of tri-methylaluminum solution (2 M in hexane, 11.0 mmol, 5 eq.). The title compound is isolated by flash chromatography on silica gel (mobile phase: dichloromethane).

Yield: 612 mg (44% of theory)

HPLC (Method 8): Rₜ=5.05 min;

MS (ESIpos): m/z=598 [M+H]⁺;

¹H-NMR (400 MHz, DMSO-d₆): δ=12.00 (s, 1H), 10.41 (s, 1H), 8.67 (s, 1H), 8.10 (d, 1H), 7.64 (d, 1H), 7.61 (d, 1H), 7.39 (d, 2H), 7.30 (d, 1H), 6.59 (d, 2H), 5.50 (t, 1H), 3.70 (t, 2H), 3.13 (qd, 2H), 0.86 (s, 9H), 0.03 (s, 6H).

### Step b): N-[2-[({4-[(2-{[tert-Butyl(dimethyl)silyl]oxy}ethyl)(cyano)amino]phenyl}amino)-carbonyl]-5-(trifluoromethyl)phenyl]-5-chlorothiophene-2-carboxamide

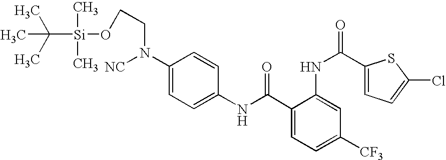

According to General Method 3, 612 mg (1.02 mmol) of N-[2-[({4-[(2-{[tert-butyl(dimethyl)-silyl]oxy}ethyl)amino]phenyl}amino)carbonyl]-5-(trifluoromethyl)phenyl]-5-chlorothiophene-2-carboxamide are reacted with a total of 545 μl of cyanogen bromide solution (3 M in dichloromethane, 1.64 mmol, 1.6 eq.) in the presence of 258 mg (3.07 mmol, 3 eq.) of sodium bicarbonate. The title compound is isolated by trituration of the crude product with diisopropyl ether.

Yield: 436 mg (66% of theory)

HPLC (Method 8): Rₜ=3.56 min;

MS (ESIpos): m/z=623 [M+H]⁺;

¹H-NMR (400 MHz, DMSO-d₆): δ=11.53 (s, 1H), 10.71 (s, 1H), 8.54 (s, 1H), 8.09 (d, 1H), 7.77 (d, 2H), 7.71 (d, 1H), 7.69 (d, 1H), 7.31 (d, 1H), 7.22 (d, 2H), 3.89-3.80 (m, 4H), 0.86 (s, 9H), 0.0 (s, 6H).

### Step c): 5-Chloro-N-[2-({[4-(2-imino-1,3-oxazolidin-3-yl)phenyl]amino}carbonyl)-5-(tri-fluoromethyl)phenyl]thiophene-2-carboxamide methanesulfonate

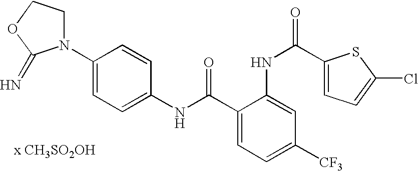

According to General Method 4, 436 mg (0.70 mmol) of N-[2-[({4-[(2-{[tert-butyl(di-methyl)silyl]oxy}ethyl)(cyano)amino]phenyl}amino)carbonyl]-5-(trifluoromethyl)phenyl]-5-chlorothiophene-2-carboxamide are reacted with 95 μl (1.47 mmol, 2.1 eq.) of methanesulfonic acid. The title compound is isolated by filtration of the precipitate formed, washing with acetonitrile and drying under reduced pressure.

Yield: 181 mg (43% of theory)

HPLC (Method 9): Rₜ=4.48 min;

MS (ESIpos): m/z=509 [M+H]⁺ (free base);

¹H-NMR (400 MHz, DMSO-d₆): δ=11.31 (s, 1H), 10.81 (s, 1H), 9.56 (br. s, 1H), 8.84 (br. s, 1H), 8.39 (s, 1H), 8.04 (d, 1H), 7.88 (d, 2H), 7.73 (d, 1H), 7.70 (d, 1H), 7.52 (d, 2H), 7.31 (d, 1H), 4.86 (t, 2H), 4.25 (t, 2H), 2.30 (s, 3H).

### Example 4

### 5-Chloro-N-[5-chloro-2-({[4-(2-imino-1,3-oxazolidin-3-yl)phenyl]amino}carbonyl)phenyl]thiophene-2-carboxamide methanesulfonate

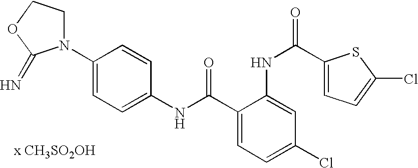

### Step a): N-{2-[({4-[(2-{[tert-Butyl(dimethyl)silyl]oxy}ethyl)amino]phenyl}amino)-carbonyl]-5-chlorophenyl}-5-chlorothiophene-2-carboxamide

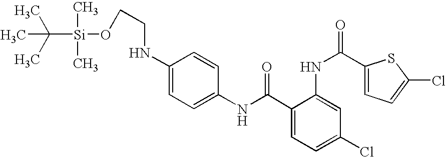

According to General Method 2, 700 mg (2.12 mmol) of the compound of Example 8A and 1.13 g (4.24 mmol, 2 eq.) of the compound of Example 3A are reacted in the presence of 5.3 ml of tri-methylaluminum solution (2 M in hexane, 10.6 mmol, 5 eq.). The title compound is isolated by preparative RP-HPLC of the crude product.

Yield: 541 mg (45% of theory)

LC-MS (Method 1): Rₜ=3.44 min;

MS (ESIpos): m/z=564 [M+H]⁺.

### Step b): N-{2-[({4-[(2-{[tert-Butyl(dimethyl)silyl]oxy}ethyl)(cyano)amino]phenyl}-amino)carbonyl]-5-chlorophenyl}-5-chlorothiophene-2-carboxamide

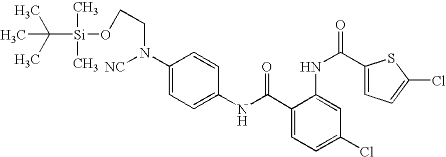

According to General Method 3, 140 mg (0.25 mmol) of N-{2-[({4-[(2-{[tert-butyl(di-methyl)silyl]oxy}ethyl)amino]phenyl}amino)carbonyl]-5-chlorophenyl}-5-chlorothiophene-2-carboxamide are reacted with a total of 124 μl of cyanogen bromide solution (3 M in dichloromethane, 0.37 mmol, 1.5 eq.) in the presence of 62 mg (0.74 mmol, 3 eq.) of sodium bicarbonate. The title compound is isolated by trituration of the crude product with diisopropyl ether.

Yield: 116 mg (79% of theory)

LC-MS (Method 3): Rₜ=3.56 min;

MS (ESIpos): m/z=589 [M+H]⁺.

Step c): 5-Chloro-N-[5-chloro-2-({[4-(2-imino-1,3-oxazolidin-3-yl)phenyl]amino}carbonyl)phenyl]thiophene-2-carboxamide methanesulfonate

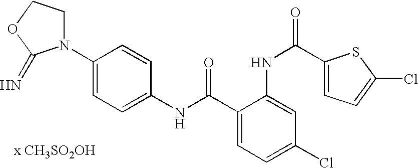

According to General Method 4, 116 mg (0.20 mmol) of N-{2-[({4-[(2-{[tert-butyl(dimethyl)silyl]-oxy}ethyl)(cyano)amino]phenyl}amino)carbonyl]-5-chlorophenyl}-5-chlorothiophene-2-carboxamide are reacted with 27 μl (0.41 mmol, 2.1 eq.) of methanesulfonic acid. The reaction mixture is concentrated under reduced pressure and the title compound is isolated by trituration of the crude product with diethyl ether.

Yield: 94 mg (84% of theory)

LC-MS (Method 1): Rₜ=1.71 min;

MS (ESIpos): m/z=475 [M+H]⁺ (free base);

¹H-NMR (400 MHz, DMSO-d₆): δ=11.58 (s, 1H), 10.80 (s, 1H), 9.60 (br. s, 1H), 8.83 (br. s, 1H), 8.28 (s, 1H), 7.92 (d, 1H), 7.86 (d, 2H), 7.65 (d, 1H), 7.52 (d, 2H), 7.42 (d, 1H), 7.31 (d, 1H), 4.86 (t, 2H), 4.24 (t, 2H), 2.36 (s, 3H).

### Example 5

### 4-Chloro-2-[(4-chlorobenzoyl)amino]-N-[4-(2-imino-1,3-oxazolidin-3-yl)phenyl]benzamide methanesulfonate

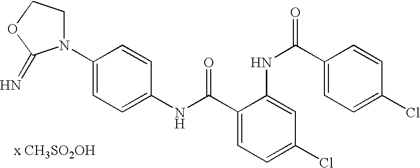

### Step a): N-{4-[(2-{[tert-Butyl(dimethyl)silyl]oxy}ethyl)amino]phenyl}-4-chloro-2-[(4-chlorobenzoyl)amino]benzamide

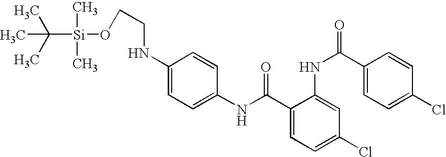

According to General Method 2, 300 mg (0.93 mmol) of the compound of Example 9A and 493 mg (1.85 mmol, 2 eq.) of the compound of Example 3A are reacted in the presence of 2.3 ml of trimethylaluminum solution (2 M in hexane, 4.6 mmol, 5 eq.). The title compound is isolated by preparative RP-HPLC of the crude product.

Yield: 291 mg (55% of theory)

LC-MS (Method 2): Rₜ=3.66 min;

MS (ESIpos): m/z=558 [M+H]⁺;

¹H-NMR (400 MHz, DMSO-d₆): δ=12.28 (s, 1H), 10.28 (s, 1H), 8.62 (s, 1H), 7.93 (d, 1H), 7.88 (d, 2H), 7.62 (d, 2H), 7.31 (d, 3H), 6.55 (d, 2H), 5.47 (t, 1H), 3.68 (t, 2H), 3.11 (qd, 2H), 0.82 (s, 9H), 0.0 (s, 6H).

### Step b): N-{4-[(2-{[tert-Butyl(dimethyl)silyl]oxy}ethyl) (cyano)amino]phenyl}-4-chloro-2-[(4-chlorobenzoyl)amino]benzamide

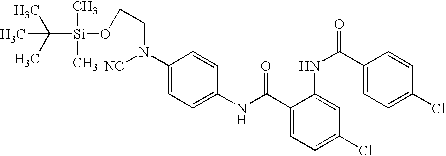

According to General Method 3, 291 mg (0.52 mmol) of N-{4-[(2-{[tert-butyl(dimethyl)-silyl]oxy}ethyl)amino]phenyl}-4-chloro-2-[(4-chlorobenzoyl)amino]benzamide are reacted with a total of 208 μl of cyanogen bromide solution (3 M in dichloromethane, 0.63 mmol, 1.2 eq.) in the presence of 131 mg (1.56 mmol, 3 eq.) of sodium bicarbonate. The title compound is isolated by trituration of the crude product with diethyl ether.

Yield: 167 mg (50% of theory)

LC-MS (Method 1): Rₜ=3.34 min;

MS (ESIpos): m/z=583 [M+H]⁺.

### Step c): 4-Chloro-2-[(4-chlorobenzoyl)amino]-N-[4-(2-imino-1,3-oxazolidin-3-yl)phenyl]-benzamide methanesulfonate

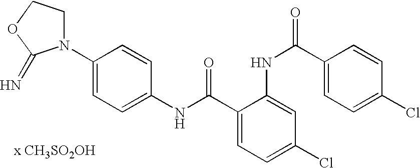

According to General Method 4, 135 mg (0.23 mmol) of N-{4-[(2-{[tert-butyl(dimethyl)silyl]-oxy}ethyl)(cyano)amino]phenyl}-4-chloro-2-[(4-chlorobenzoyl)amino]benzamide are reacted with 32 μl (0.49 mmol, 2.1 eq.) of methanesulfonic acid. The title compound is isolated by filtration of the precipitate formed, washing with acetonitrile and drying under reduced pressure.

Yield: 131 mg (93% of theory)

LC-MS (Method 1): Rₜ=1.71 min;

MS (ESIpos): m/z=469 [M+H]⁺ (free base);

¹H-NMR (400 MHz, DMSO-d₆): δ=11.61 (s, 1H), 10.80 (s, 1H), 9.59 (br. s, 1H), 8.82 (br. s, 1H), 8.43 (s, 1H), 7.98-7.88 (m, 3H), 7.86 (d, 2H), 7.68 (d, 2H), 7.51 (d, 2H), 7.43 (d, 1H), 4.86 (t, 2H), 4.23 (t, 2H), 2.32 (s, 3H).

### Example 6

### 5-Chloro-N-[5-fluoro-2-({[4-(2-imino-1,3-oxazolidin-3-yl)phenyl]amino}carbonyl)phenyl]thiophene-2-carboxamide methanesulfonate

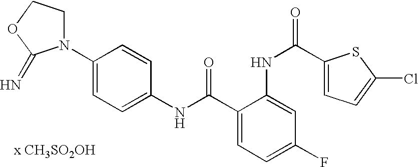

### Step a): N-{2-[({4-[(2-{[tert-Butyl(dimethyl)silyl]oxy}ethyl)amino]phenyl}amino)-carbonyl]-5-fluorophenyl)}-5-chlorothiophene-2-carboxamide

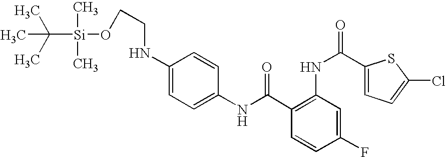

According to General Method 2, 302 mg (0.96 mmol) of the compound of Example 10A and 513 mg (1.93 mmol, 2 eq.) of the compound of Example 3A are reacted in the presence of 2.4 ml of trimethylaluminum solution (2 M in hexane, 4.81 mmol, 5 eq.).

Yield: 344 mg (87% purity, 65% of theory)

LC-MS (Method 1): Rₜ=3.38 min;

MS (ESIpos): m/z=548 [M+H]⁺;

¹H-NMR (400 MHz, DMSO-d₆): δ=12.53 (s, 1H), 10.28 (s, 1H), 8.29 (dd, 1H), 8.07 (dd, 1H), 7.58 (d, 1H), 7.38 (d, 2H), 7.30 (d, 1H), 7.18-7.10 (m, 1H), 6.61 (d, 2H), 5.50 (t, 1H), 3.72 (t, 2H), 3.16 (q, 2H), 0.88 (s, 9H), 0.05 (s, 6H).

### Step b): N-{2-[({4-[(2-{[tert-Butyl(dimethyl)silyl]oxy}ethyl)(cyano)amino]phenyl}amino)-carbonyl]-5-fluorophenyl}-5-chlorothiophene-2-carboxamide

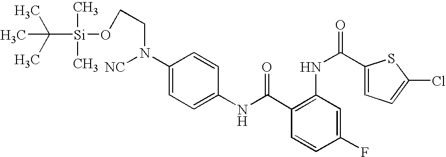

According to General Method 3, 344 mg (0.63 mmol) of N-{2-[({4-[(2-{[tert-butyl(dimethyl)-silyl]oxy}ethyl)amino]phenyl}amino)carbonyl]-5-fluorophenyl}-5-chlorothiophene-2-carboxamide are reacted with a total of 0.25 ml of cyanogen bromide solution (3 M in dichloromethane, 0.75 mmol, 1.2 eq.) in the presence of 158 mg (1.88 mmol, 3 eq.) of sodium bicarbonate. The title compound is isolated by trituration of the crude product with diethyl ether.

Yield: 330 mg (77% purity, 71% of theory)

LC-MS (Method 1): Rₜ=3.50 min;

MS (ESIpos): m/z=573 [M+H]⁺.

### Step c): 5-Chloro-N-[5-fluoro-2-({[4-(2-imino-1,3-oxazolidin-3-yl)phenyl]amino}carbonyl)phenyl]thiophene-2-carboxamide methanesulfonate

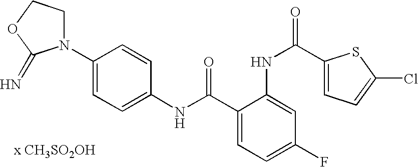

According to General Method 4, 328 mg (0.44 mmol) of N-{2-[({4-[(2-{[tert-butyl(dimethyl)-silyl]oxy}ethyl)(cyano)amino]phenyl}amino)carbonyl]-5-fluorophenyl}-5-chlorothiophene-2-carboxamide (77% pure) are reacted with 63 μl (0.97 mmol, 2.2 eq.) of methanesulfonic acid. The title compound is isolated by filtration of the precipitate formed, washing with acetonitrile and drying under reduced pressure.

Yield: 10 mg (4% of theory)

LC-MS (Method 1): Rₜ=1.61 min;

MS (ESIpos): m/z=459 [M+H]⁺ (free base);

¹H-NMR (400 MHz, DMSO-d₆): δ=11.83 (s, 1H), 10.76 (s, 1H), 9.60 (br. s, 1H), 8.85 (br. s, 1H), 8.12 (d, 1H), 8.02 (t, 3H), 7.87 (d, 2H), 7.63 (d, 1H), 7.53 (d, 2H), 7.32 (d, 1H), 7.21 (t, 1H), 4.86 (t, 2H), 4.24 (t, 2H), 2.30 (s, 3H).

### Example 7

### 5-Chloro-N-[2-({[4-(2-imino-1,3-oxazolidin-3-yl)phenyl]amino}carbonyl)-5-methoxyphenyl]thiophene-2-carboxamide methanesulfonate

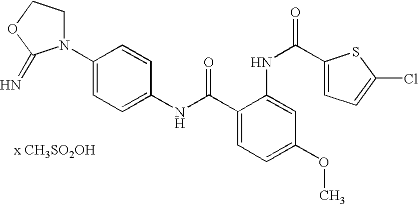

### Step a): N-{2-[({4-[(2-{[tert-Butyl(dimethyl)silyl]oxy}ethyl)amino]phenyl}amino)-carbonyl]-5-methoxyphenyl}-5-chlorothiophene-2-carboxamide

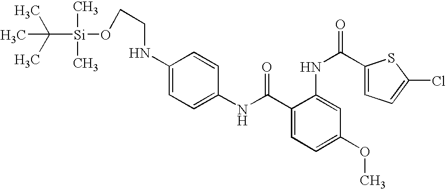

According to General Method 2, 300 mg (0.9 mmol) of the compound of Example 11A and 491 mg (1.8 mmol, 2 eq.) of the compound of Example 3A are reacted in the presence of 2.3 ml of trimethylaluminum solution (2 M in hexane, 4.62 mmol, 5 eq.). The title compound is isolated by preparative RP-HPLC of the crude product.

Yield: 185 mg (38% of theory)

LC-MS (Method 1): Rₜ=3.36 min;

MS (ESIpos): m/z=560 [M+H]⁺.

### Step b): N-{2-[({4-[(2-{[tert-Butyl(dimethyl)silyl]oxy}ethyl)(cyano)amino]phenyl}amino)-carbonyl]-5-methoxyphenyl}-5-chlorothiophene-2-carboxamide

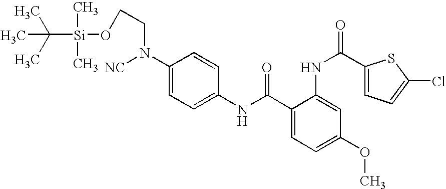

According to General Method 3, 190 mg (0.3 mmol) of N-{2-[({4-[(2-{[tert-butyl(dimethyl)-silyl]oxy}ethyl)amino]phenyl}amino)carbonyl]-5-methoxyphenyl}-5-chlorothiophene-2-carboxamide are reacted with a total of 170 μl of cyanogen bromide solution (3 M in dichloromethane, 0.51 mmol, 1.5 eq.) in the presence of 85 mg (1.02 mmol, 3 eq.) of sodium bicarbonate. The title compound is isolated by preparative RP-HPLC of the crude product.

Yield: 38 mg (88% purity, 17% of theory)

LC-MS (Method 2): Rₜ=3.53 min;

MS (ESIpos): m/z=585 [M+H]⁺;

¹H-NMR (400 MHz, DMSO-d₆): δ=12.43 (s, 1H), 10.43 (s, 1H), 8.11 (d, 1H), 8.00 (d, 1H), 7.73 (d, 2H), 7.58 (d, 1H), 7.31 (d, 1H), 7.22 (d, 2H), 6.88 (dd, 1H), 3.94-3.83 (m, 4H), 3.86 (s, 3H), 0.85 (s, 9H), 0.0 (s, 6H).

### Step c): 5-Chloro-N-[2-({[4-(2-imino-1,3-oxazolidin-3-yl)phenyl]amino}carbonyl)-5-methoxyphenyl]thiophene-2-carboxamide methanesulfonate

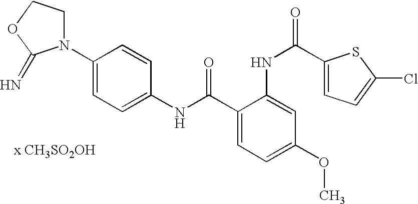

According to General Method 4, 35 mg (0.06 mmol) of N-{2-[({4-[(2-{[tert-butyl(dimethyl)-silyl]oxy}ethyl) (cyano)amino]phenyl}amino)carbonyl]-5-methoxyphenyl}-5-chlorothiophene-2-carboxamide are reacted with 9 μl (0.13 mmol, 2.2 eq.) of methanesulfonic acid. The title compound is isolated by filtration of the precipitate formed, washing with acetonitrile and diethyl ether and drying under reduced pressure.

Yield: 27 mg (76% of theory)

LC-MS (Method 1): Rₜ=1.61 min;

MS (ESIpos): m/z=471 [M+H]⁺ (free base);

¹H-NMR (400 MHz, DMSO-d₆): δ=12.18 (s, 1H), 10.61 (s, 1H), 9.58 (br. s, 1H), 8.84 (br. s, 1H), 8.04 (s, 1H), 7.97 (s, 1H), 7.87 (d, 2H), 7.59 (s, 1H), 7.52 (d, 2H), 7.32 (d, 1H), 6.89 (d, 1H), 4.86 (t, 2H), 4.25 (t, 2H), 3.86 (s, 3H), 2.36 (s, 3H).

### Example 8

### 5-Chloro-N-[5-cyano-2-({[4-(2-imino-1,3-oxazolidin-3-yl)phenyl]amino}carbonyl)phenyl]thiophene-2-carboxamide methanesulfonate

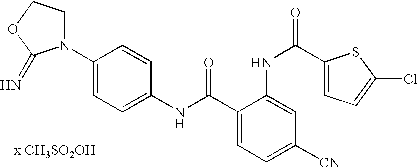

### Step a): N-{2-[({4-[(2-{[tert-Butyl(dimethyl)silyl]oxy}ethyl)amino]phenyl}amino)-carbonyl]-5-cyanophenyl}-5-chlorothiophene-2-carboxamide

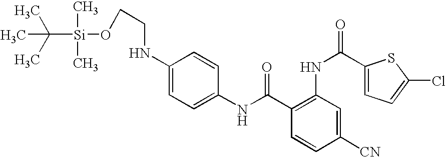

According to General Method 2, 321 mg (1.00 mmol) of the compound of Example 12A and 533 mg (2.00 mmol, 2 eq.) of the compound of Example 3A are reacted in the presence of 2.5 ml of trimethylaluminum solution (2 M in hexane, 5.00 mmol, 5 eq.). The title compound is isolated by preparative RP-HPLC of the crude product.

Yield: 323 mg (58% of theory)

LC-MS (Method 2): Rₜ=3.58 min;

MS (ESIpos): m/z=555 [M+H]⁺.

### Step b): N-{2-[({4-[(2-{[tert-Butyl(dimethyl)silyl]oxy}ethyl)(cyano)amino]phenyl}-amino)carbonyl]-5-cyanophenyl}-5-chlorothiophene-2-carboxamide

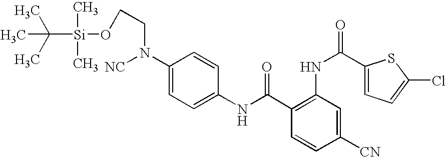

According to General Method 3, 320 mg (0.58 mmol) of N-{2-[({4-[(2-{[tert-butyl(dimethyl)-silyl]oxy}ethyl)amino]phenyl}amino)carbonyl]-5-cyanophenyl}-5-chlorothiophene-2-carboxamide are reacted with a total of 231 μl of cyanogen bromide solution (3 M in dichloromethane, 0.70 mmol, 1.2 eq.) in the presence of 145 mg (1.73 mmol, 3 eq.) of sodium bicarbonate. The title compound is isolated by silica gel chromatography (mobile phase: cyclohexane/ethyl acetate 1:1).

Yield: 330 mg (95% of theory)

LC-MS (Method 3): Rₜ=3.42 min;

MS (ESIpos): m/z=580 [M+H]⁺;

¹H-NMR (400 MHz, DMSO-d₆): δ=11.42 (s, 1H), 10.72 (s, 1H), 8.51 (d, 1H), 8.04 (d, 1H), 7.82 (d, 1H), 7.75 (d, 2H), 7.70 (d, 1H), 7.31 (d, 1H), 7.22 (d, 2H), 3.85-3.84 (m, 4H), 0.86 (s, 9H), 0.01 (s, 6H).

### Step c): 5-Chloro-N-[5-cyano-2-({[4-(2-imino-1,3-oxazolidin-3-yl)phenyl]amino}carbonyl)phenyl]thiophene-2-carboxamide methanesulfonate

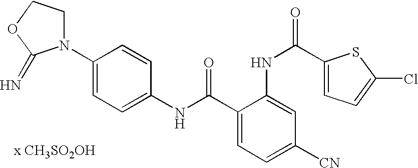

According to General Method 4, 330 mg (0.48 mmol) of N-{2-[({4-[(2-{[tert-butyl(dimethyl)-silyl]oxy}ethyl)(cyano)amino]phenyl}amino)carbonyl]-5-cyanophenyl}-5-chlorothiophene-2-carboxamide (85% pure) are reacted with 69 μl (1.06 mmol, 2.2 eq.) of methanesulfonic acid. The title compound is isolated by filtration of the precipitate formed, washing with acetonitrile/diethyl ether and drying under reduced pressure.

Yield: 260 mg (91% of theory)

LC-MS (Method 1): Rₜ=1.62 min;

MS (ESIpos): m/z=466 [M+H]⁺ (free base);

¹H-NMR (400 MHz, DMSO-d₆): δ=11.22 (s, 1H), 10.90 (s, 1H), 9.59 (br. s, 1H), 8.85 (br. s, 1H), 8.36 (br. s, 1H), 7.97 (s, 1H), 7.87-7.84 (2d, 3H), 7.72 (d, 1H), 7.51 (d, 2H), 7.31 (d, 1H), 4.86 (t, 2H), 4.24 (t, 2H), 2.31 (br. s, 3H).

### Example 9

### 3-{[(5-Chloro-2-thienyl)carbonyl]amino}-N-[4-(2-imino-1,3-oxazolidin-3-yl)phenyl]pyridine-2-carboxamide methanesulfonate

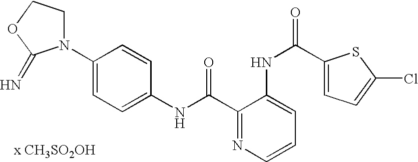

### Step a): N-{4-[(2-{[tert-Butyl(dimethyl)silyl]oxy}ethyl)amino]phenyl}-3-{[(5-chloro-2-thienyl)carbonyl]amino}pyridine-2-carboxamide

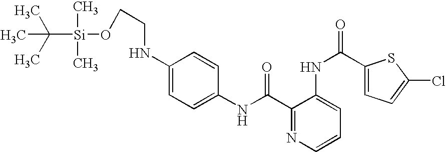

According to General Method 1, 161 mg (0.6 mmol) of the compound of Example 13A and 152 mg (0.6 mmol, 1 eq.) of the compound of Example 3A are reacted in the presence of 321 mg of TBTU (0.6 mmol, 1.05 eq.) and 104 μl of N,N-diisopropylethylamine (77 mg, 1.05 eq.). The title compound is isolated by preparative RP-HPLC of the crude product.

Yield: 156 mg (58% of theory)

HPLC (Method 8): Rₜ=5.76 min;

MS (ESIpos): nm/z=531 [M+H]⁺;

¹H-NMR (400 MHz, DMSO-d₆): δ=13.09 (s, 1H), 10.64 (s, 1H), 8.93 (dd, 1H), 8.40 (dd, 1H), 7.64 (dd, 1H), 7.60 (d, 1H), 7.50 (d, 2H), 7.30 (d, 1H), 6.56 (d, 2H), 5.48 (t, 1H), 3.66 (t, 2H), 3.11 (dt, 2H), 0.82 (s, 9H), 0.00 (s, 6H).

### Step b): N-{4-[(2-{[tert-Butyl(dimethyl)silyl]oxy)ethyl)(cyano)amino]phenyl}-3-{[(5-chloro-2-thienyl)carbonyl]amino}pyridine-2-carboxamide

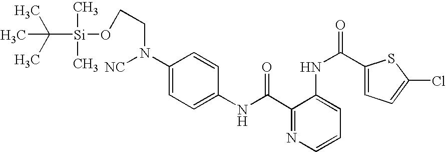

According to General Method 3, 129 mg (0.2 mmol) of N-{4-[(2-{[tert-butyl(dimethyl)-silyl]oxy}ethyl)amino]phenyl}-3-{[(5-chloro-2-thienyl)carbonyl]amino}pyridine-2-carboxamide are reacted with a total of 160 μl of cyanogen bromide solution (3 M in dichloromethane, 0.4 mmol, 2 eq.) in the presence of 61 mg (0.7 mmol, 3 eq.) of sodium bicarbonate. The crude product is reacted without any further purification.

Yield: 117 mg (81% purity, 70% of theory)

LC-MS (Method 1): Rₜ=3.38 min;

MS (ESIpos): m/z=556 [M+H]⁺.

### Step c): 3-{[(5-Chloro-2-thienyl)carbonyl]amino}-N-[4-(2-imino-1,3-oxazolidin-3-yl)-phenyl]pyridine-2-carboxamide methanesulfonate

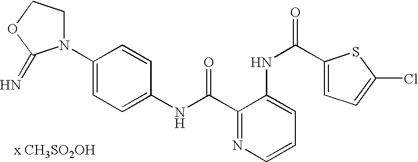

According to General Method 4, 113 mg (0.2 mmol) of N-{4-[(2-{[tert-butyl(dimethyl)-silyl]oxy}ethyl)(cyano)amino]phenyl}-3-{[(5-chloro-2-thienyl)carbonyl]amino}pyridine-2-carboxamide are reacted with 29 μl (0.4 mmol, 2.2 eq.) of methanesulfonic acid. The title compound is isolated by trituration of the crude product with diethyl ether.

Yield: 97 mg (89% of theory)

LC-MS (Method 1): Rₜ=1.67 min;

MS (ESIpos): m/z=442 [M+H]⁺ (free base);

¹H-NMR (400 MHz, DMSO-d₆): δ=12.68 (s, 1H), 11.25 (s, 1H), 9.60 (br. s, 1H), 8.97 (d, 1H), 8.88 (br. s, 1H), 8.52 (d, 1H), 8.06 (d, 2H), 7.77 (dd, 1H), 7.67 (d, 1H), 7.56 (d, 2H), 7.38 (d, 1H), 4.86 (t, 2H), 4.26 (t, 2H), 2.32 (s, 3H).

### Example 10

### 4-{[(5-Chloro-2-thienyl)carbonyl]amino}-N-[4-(2-imino-1,3-oxazolidin-3-yl)phenyl]nicotinamide methanesulfonate

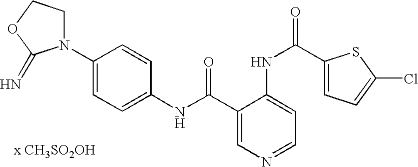

### Step a): N-{4-[(2-{[tert-Butyl(dimethyl)silyl]oxy}ethyl)amino]phenyl}-4-{[(5-chloro-2-thienyl)carbonyl]amino}nicotinamide

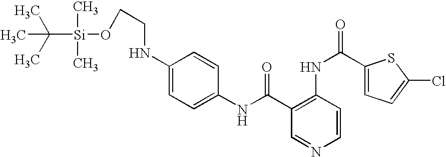

According to General Method 1, 52 mg (0.18 mmol) of the compound of Example 14A and 51 mg (0.19 mmol, 1.05 eq.) of the compound of Example 3A are reacted in the presence of 62 mg (0.19 mmol, 1.05 eq.) of TBTU and 34 μl (0.19 mmol, 1.05 eq.) of N,N-diisopropylethylamine. The title compound is isolated by preparative RP-HPLC of the crude product.

Yield: 17 mg (17% of theory)

LC-MS (Method 1): Rₜ=3.19 min;

MS (ESIpos): m/z=531 [M+H]⁺;

¹H-NMR (400 MHz, DMSO-d₆): δ=12.39 (s, 1H), 10.45 (s, 1H), 9.07 (s, 1H), 8.64 (d, 1H), 8.38 (d, 1H), 7.61 (d, 2H), 7.41 (d, 1H), 7.33 (d, 2H), 6.61 (d, 1H), 5.53 (t, 1H), 3.72 (t, 2H), 3.16 (q, 2H), 0.88 (s, 9H), 0.05 (s, 6H).

### Step b): N-{4-[(2-{[tert-Butyl(dimethyl)silyl]oxy}ethyl)(cyano)amino]phenyl}-4-{[(5-chloro-2-thienyl)carbonyl]amino}nicotinamide

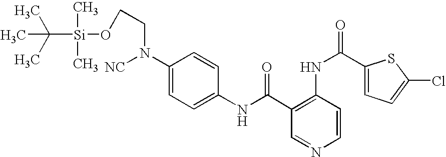

According to General Method 3, 500 mg (0.67 mmol) of N-{4-[(2-{[tert-butyl(dimethyl)-silyl]oxy}ethyl)amino]phenyl}-4-{[(5-chloro-2-thienyl)carbonyl]amino}nicotinamide are reacted with a total of 266 μl of cyanogen bromide solution (3 M in dichloromethane, 0.80 mmol, 1.2 eq.) in the presence of 168 mg (2.00 mmol, 3 eq.) of sodium bicarbonate. The title compound is isolated by preparative RP-HPLC of the crude product.

Yield: 140 mg (36% of theory)

LC-MS (Method 1): Rₜ=3.09 min;

MS (ESIpos): m/z=556 [M+H]⁺.

### Step c): 4-{[(5-Chloro-2-thienyl)carbonyl]amino}-N-[4-(2-imino-1,3-oxazolidin-3-yl)-phenyl]nicotinamide methanesulfonate

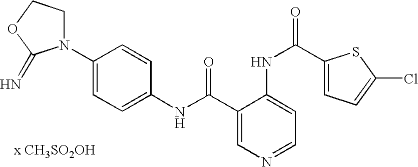

According to General Method 4, 80 mg (0.14 mmol) of N-{4-[(2-{[tert-butyl(dimethyl)silyl]-oxy}ethyl)(cyano)amino]phenyl}-4-{[(5-chloro-2-thienyl)carbonyl]amino}nicotinamide are reacted with 20 μl (0.30 mmol, 2.2 eq.) of methanesulfonic acid. The reaction mixture is concentrated under reduced pressure and the residue is triturated with diethyl ether. The solid is filtered off, washed with acetone and dried under reduced pressure.

Yield: 56 mg (75% of theory)

LC-MS (Method 3): Rₜ=1.57 min;

MS (ESIpos): m/z=442 [M+H]⁺ (free base);

¹H-NMR (400 MHz, DMSO-d₆): δ=11.86 (s, 1H), 10.98 (s, 1H), 9.60 (br. s, 1H), 9.10 (s, 1H), 8.86 (br. s, 1H), 8.74 (d, 1H), 8.34 (d, 1H), 7.88 (d, 2H), 7.69 (d, 1H), 7.54 (d, 2H), 7.36 (d, 1H), 4.86 (t, 2H), 4.25 (t, 2H), 2.31 (s, 3H).

### Example 11

### 3-{[(5-Chloro-2-thienyl)carbonyl]amino}-N-[4-(2-imino-1,3-oxazolidin-3-yl)phenyl]isonicotinamide methanesulfonate

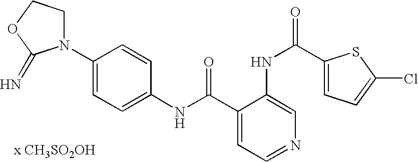

### Step a): N-{4-[(2-{[tert-Butyl(dimethyl)silyl]oxy}ethyl)amino]phenyl}-3-{[(5-chloro-2-thienyl)carbonyl]amino} isonicotinamide

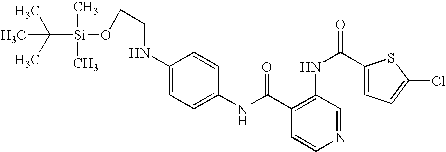

According to General Method 1, 200 mg (0.71 mmol) of the compound of Example 15A and 189 mg (0.71 mmol, 1.00 eq.) of the compound of Example 3A are reacted in the presence of 239 mg (0.74 mmol, 1.05 eq.) of TBTU and 129 μl (0.74 mmol, 1.05 eq.) of N,N-diisopropylethylamine. The title compound is isolated by preparative RP-HPLC of the crude product.

Yield: 164 mg (44% of theory)

LC-MS (Method 1): Rₜ=3.15 min;

MS (ESIpos): m/z=531 [M+H]⁺;

¹H-NMR (400 MHz, DMSO-d₆): δ=11.29 (s, 1H), 10.31 (s, 1H), 9.23 (s, 1H), 8.49 (d, 1H), 7.73 (d, 1H), 7.65 (d, 1H), 7.34 (d, 2H), 7.25 (d, 1H), 6.54 (d, 2H), 5.45 (t, 1H), 3.67 (t, 2H), 3.10 (q, 2H), 0.83 (s, 9H), 0.00 (s, 6H).

### Step b): N-{4-[(2-{[tert-Butyl(dimethyl)silyl]oxy}ethyl)(cyano)amino]phenyl}-3-{[(5-chloro-2-thienyl)carbonyl]amino}isonicotinamide

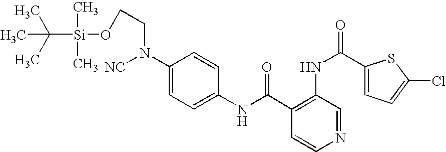

According to General Method 3, 2.70 g (5.01 mmol) of N-{4-[(2-{[tert-butyl(dimethyl)silyl]-oxy}ethyl)amino]phenyl}-3-{[(5-chloro-2-thienyl)carbonyl]amino} isonicotinamide are reacted with a total of 2.0 ml of cyanogen bromide solution (3 M in dichloromethane, 6.10 mmol, 1.2 eq.) in the presence of 1.28 g (15.25 mmol, 3 eq.) of sodium bicarbonate. The title compound is isolated by trituration of the crude product with diethyl ether.

Yield: 750 mg (91% purity, 24% of theory)

LC-MS (Method 1): Rₜ=2.97 min;

MS (ESIpos): m/z=556 [M+H]⁺;

¹H-NMR (400 MHz, DMSO-d₆): δ=12.20 (s, 1H), 10.09 (s, 1H), 7.69-7.66 (2d, 2H), 7.61 (d, 1H), 7.30 (d, 1H), 7.20 (d, 2H), 6.79 (d, 1H), 6.73 (d, 1H), 6.16 (d, 1H), 3.84 (d, 4H), 0.86 (s, 9H), 0.02 (s, 6H).

### Step c): 3-{[(5-Chloro-2-thienyl)carbonyl]amino}-N-[4-(2-imino-1,3-oxazolidin-3-yl)-phenyl]isonicotinamide methanesulfonate

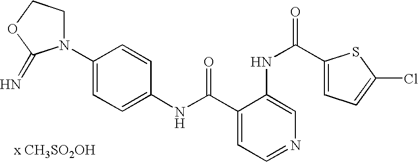

According to General Method 4, 2.02 g (3.64 mmol) of N-{4-[(2-{[tert-butyl(dimethyl)-silyl]oxy}ethyl)(cyano)amino]phenyl}-3-{[(5-chloro-2-thienyl)carbonyl]amino}isonicotinamide are reacted with 519 μl (8.00 mmol, 2.2 eq.) of methanesulfonic acid. The title compound is isolated by filtration of the precipitate formed, washing with acetonitrile/diethyl ether and drying under reduced pressure.

Yield: 2.00 g (98% of theory)

LC-MS (Method 1): Rₜ=1.23 min;

MS (ESIpos): m/z=442 [M+H]⁺ (free base);

¹H-NMR (400 MHz, DMSO-d₆): δ=10.89 (s, 1H), 10.85 (s, 1H), 9.57 (br. s, 1H), 8.97 (s, 1H), 8.82 (br. s, 1H), 8.61 (d, 1H), 7.84 (d, 2H), 7.79 (d, 1H), 7.71 (d, 1H), 7.50 (d, 2H), 7.30 (d, 1H), 4.85 (t, 2H), 4.23 (t, 2H), 2.32 (s, 3H).

### Example 12

### 3-[(4-Chlorobenzoyl)amino]-N-[4-(2-imino-1,3-oxazolidin-3-yl)phenyl]isonicotinamide methanesulfonate

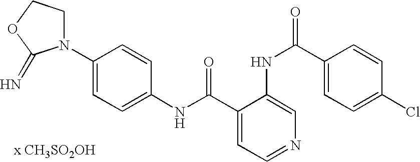

### Step a): N-{4-[(2-{[tert-Butyl(dimethyl)silyl]oxy}ethyl)amino]phenyl}-3-[(4-chloro-benzoyl)amino]isonicotinamide

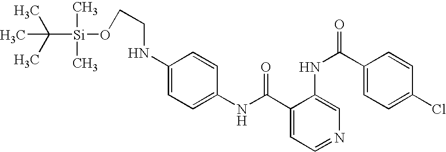

According to General Method 2, 292 mg (1.0 mmol) of the compound of Example 16A and 535 mg (2.0 mmol, 2 eq.) of the compound of Example 3A are reacted in the presence of 2.5 ml of trimethylaluminum solution (2 M in hexane, 5.0 mmol, 5 eq.). The title compound is isolated by preparative RP-HPLC of the crude product.

Yield: 421 mg (80% of theory)

LC-MS (Method 3): Rₜ=3.27 min;

MS (ESIpos): m/z=525 [M+H]⁺.

### Step b): N-{4-[(2-{[tert-Butyl(dimethyl)silyl]oxy}ethyl)(cyano)amino]phenyl}-3-[(4-chlorobenzoyl)amino]isonicotinamide

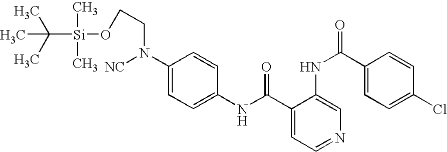

According to General Method 3, 208 mg (0.40 mmol) of N-{4-[(2-{[tert-butyl(dimethyl)silyl]-oxy}ethyl)amino]phenyl}-3-[(4-chlorobenzoyl)amino]isonicotinamide are reacted with a total of 158 μl of cyanogen bromide solution (3 M in dichloromethane, 0.48 mmol, 1.2 eq.) in the presence of 100 mg (1.19 mmol, 3 eq.) of sodium bicarbonate. The title compound is isolated by flash chromatography on silica gel (mobile phase: dichloromethane/methanol 100:1→100:2).

Yield: 39 mg (18% of theory)

LC-MS (Method 1): Rₜ=2.97 min;

MS (ESIpos): m/z=550 [M+H]⁺.

### Step c): 3-[(4-Chlorobenzoyl)amino]-N-[4-(2-imino-1,3-oxazolidin-3-yl)phenyl]isonicotin-amide methanesulfonate

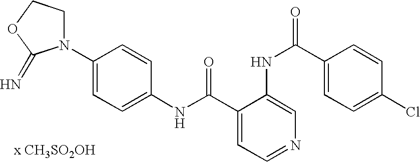

According to General Method 4, 39 mg (0.07 mmol) of N-{4-[(2-{[tert-butyl(dimethyl)-silyl]oxy}ethyl)(cyano)amino]phenyl}-3-[(4-chlorobenzoyl)amino]isonicotinamide are reacted with 10 μl (0.15 mmol, 2.1 eq.) of methanesulfonic acid. The title compound is isolated by trituration of the crude product with diethyl ether.

Yield: 16 mg (43% of theory)

LC-MS (Method 2): Rₜ=1.48 min;

MS (ESIpos): m/z=436 [M+H]⁺ (free base);

¹H-NMR (400 MHz, DMSO-d₆): δ=11.01 (s, 1H), 10.92 (s, 1H), 9.59 (br. s, 1H), 9.11 (s, 1H), 8.81 (br. s, 1H), 8.67 (d, 1H), 7.93 (d, 2H), 7.88-7.80 (2d, 3H), 7.65 (d, 2H), 7.50 (d, 2H), 4.83 (t, 2H), 4.22 (t, 2H), 2.38 (s, 3H).

### Example 13

### 2-{([(5-Chloro-2-thienyl)carbonyl]amino}-N-[4-(2-imino-1,3-oxazolidin-3-yl)phenyl]nicotinamide methanesulfonate

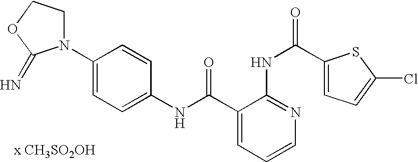

### Step a): N-{4-[(2-{[tert-Butyl(dimethyl)silyl]oxy}ethyl)amino]phenyl}-2-{[(5-chloro-2-thienyl)carbonyl]amino}nicotinamide

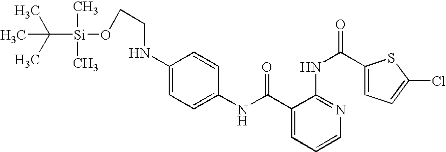

According to General Method 1, 330 mg (1.0 mmol, 90% pure) of the compound of Example 17A and 267 mg (1.0 mmol, 1.05 eq.) of the compound of Example 3A are reacted in the presence of 337 mg of TBTU (1.1 mmol, 1.1 eq.) and 183 μl of N,N-diisopropylethylamine (134 mg, 1.1 eq.). After a reaction time of 16 hours, the same amounts of TBTU and N,N-diisopropylethylamine are added again. After a total of 48 hours, the reaction is worked up as described. The title compound is isolated by preparative RP-HPLC of the crude product.

Yield: 33 mg (7% of theory)

LC-MS (Method 3): Rₜ=3.23 min;

MS (ESIpos): m/z=531 [M+H]⁺;

¹H-NMR (400 MHz, DMSO-d₆): δ=11.20 (s, 1H), 10.01 (s, 1H), 8.40 (dd, 1H), 8.04 (dd, 1H), 7.86 (d, 1H), 7.33 (dd, 1H), 7.27 (d, 2H), 7.22 (d, 1H), 6.50 (d, 2H), 5.32 (br. s, 1H), 3.66 (t, 2H), 3.09 (t, 2H), 0.83 (s, 9H), 0.00 (s, 6H).

### Step b): N-{4-[(2-{[tert-Butyl(dimethyl)silyl]oxy}ethyl)(cyano)amino]phenyl}-2-{[(5-chloro-2-thienyl)carbonyl]amino}nicotinamide

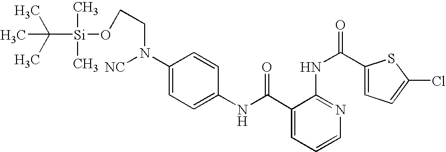

According to General Method 3, 30 mg (0.06 mmol) of N-{4-[(2-{[tert-butyl(dimethyl)-silyl]oxy}ethyl)amino]phenyl}-2-{[(5-chloro-2-thienyl)carbonyl]amino}nicotinamide are reacted with a total of 28 μl of cyanogen bromide solution (3 M in dichloromethane, 0.09 mmol, 1.5 eq.) in the presence of 14 mg (0.17 mmol, 3 eq.) of sodium bicarbonate. The crude product is reacted without any further purification.

Yield: 27 mg (85% purity, 73% of theory)

LC-MS (Method 1): Rₜ=2.95 min;

MS (ESIpos): m/z=556 [M+H]⁺.

### Step c): 2-{[(5-Chloro-2-thienyl)carbonyl]amino}-N-[4-(2-imino-1,3-oxazolidin-3-yl)-phenyl]nicotinamide methanesulfonate

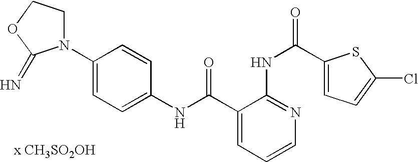

According to General Method 4, 25 mg (0.04 mmol, 85% pure) of N-{4-[(2-{[tert-butyl-(dimethyl)silyl]oxy}ethyl)(cyano)amino]phenyl}-2-{[(5-chloro-2-thienyl)carbonyl]amino}-nicotinamide are reacted with 5 μl (0.08 mmol, 2.2 eq.) of methanesulfonic acid. The title compound is isolated by trituration of the crude product with diethyl ether.

Yield: 20 mg (93% of theory)

LC-MS (Method 2): Rₜ=1.57 min;

MS (ESIpos): m/z=442 [M+H]⁺ (free base);

¹H-NMR (400 MHz, DMSO-d₆): δ=11.20 (s, 1H), 10.71 (s, 1H), 9.55 (br. s, 1H), 8.81 (br. s, 1H), 8.58 (d, 1H), 8.05 (d, 1H), 7.99 (d, 1H), 7.81 (d, 2H), 7.46 (d, 2H), 7.45-7.41 (m, 1H), 4.84 (t, 2H), 4.22 (t, 2H), 2.34 (s, 3H).

### Example 14

### 3-[(4-Chlorobenzoyl)amino]-N-[4-(2-imino-1,3-oxazolidin-3-yl)phenyl]pyrazine-2-carboxamide methanesulfonate

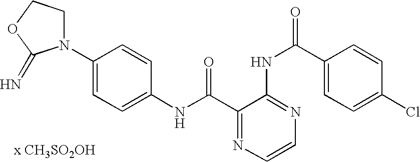

### Step a): N-{4-[(2-{[tert-Butyl(dimethyl)silyl]oxy}ethyl)amino]phenyl}-3-[(4-chlorobenzoyl)amino]pyrazine-2-carboxamide

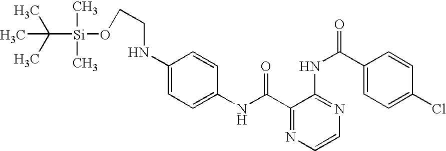

46 mg (0.17 mmol) of the compound of Example 18A are dissolved in 0.7 ml of DMF, and 15 mg (0.15 mmol, 0.9 eq.) of triethylamine and 21 mg (0.18 mmol, 11.1 eq.) of pivaloyl chloride are added. The mixture is stirred at room temperature for 1 h. 44 mg (0.17 mmol, 1.0 eq.) of the compound of Example 3A are then added, and the reaction mixture is stirred at room temperature for 16 h. The title compound is isolated by preparative RP-HPLC of the crude product.

Yield: 16 mg (18% of theory)

LC-MS (Method 1): Rₜ=3.19 min;

MS (ESIpos): m/z=526 [M+H]⁺.

### Step b): N-{4-[(2-{[tert-Butyl(dimethyl)silyl]oxy}ethyl)(cyano)amino]phenyl}-3-[(4-chlorobenzoyl)amino]pyrazine-2-carboxamide

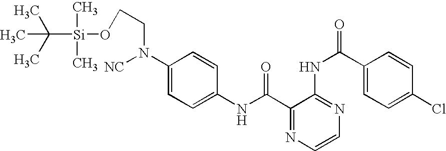

According to General Method 3, 16 mg (0.03 mmol) of N-{4-[(2-{[tert-butyl(dimethyl)silyl]oxy}-ethyl)amino]phenyl}-3-[(4-chlorobenzoyl)amino]pyrazine-2-carboxamide are reacted with a total of 12 μl of cyanogen bromide solution (3 M in dichloromethane, 0.04 mmol, 1.2 eq.) in the presence of 8 mg (0.09 mmol, 3 eq.) of sodium bicarbonate. The crude product is reacted without any further purification.

Yield: 15 mg (90% of theory)

LC-MS (Method 1): Rₜ=3.05 min;

MS (ESIpos): m/z=551 [M+H]⁺.

### Step c): 3-[(4-Chlorobenzoyl)amino]-N-[4-(2-imino-1,3-oxazolidin-3-yl)phenyl]pyrazine-2-carboxamide methanesulfonate

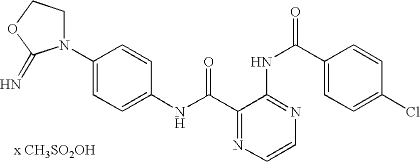

According to General Method 4, 15 mg (0.03 mmol) of N-{4-[(2-{[tert-butyl(dimethyl)silyl]-oxy}ethyl)(cyano)amino]phenyl}-3-[(4-chlorobenzoyl)amino]pyrazine-2-carboxamide are reacted with 4 μl (0.06 mmol, 2.2 eq.) of methanesulfonic acid. The title compound is isolated by trituration of the crude product with diethyl ether.

Yield: 7 mg (48% of theory)

HPLC (Method 7): Rₜ=3.89 min;

MS (ESIpos): m/z=437 [M+H]⁺ (free base);

¹H-NMR (400 MHz, DMSO-d₆): δ=10.79 (s, 1H), 9.59 (br. s, 1H), 8.83 (br. s, 1H), 8.32 (d, 1H), 8.02 (d, 2H), 7.96 (d, 1H), 7.60 (d, 2H), 7.51 (d, 2H), 7.23 (d, 2H), 4.83 (t, 2H), 4.25 (t, 2H).

### Example 15

### 4-{[(5-Chloro-2-thienyl)carbonyl]amino}-N-[4-(2-imino-1,3-oxazolidin-3-yl)phenyl]-2-methylpyrimidine-5-carboxamide methanesulfonate

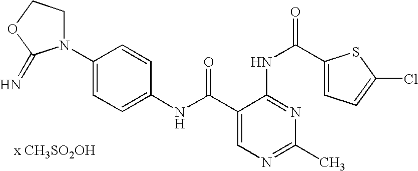

### Step a): N-{4-[(2-{[tert-Butyl(dimethyl)silyl]oxy}ethyl)amino]phenyl}-4-{[(5-chloro-2-thienyl)carbonyl]amino}-2-methylpyrimidine-5-carboxamide

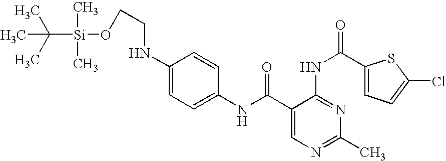

According to General Method 2, 283 mg (0.9 mmol) of the compound of Example 19A and 484 mg (1.8 mmol, 2 eq.) of the compound of Example 3A are reacted in the presence of 2.3 ml of trimethylaluminum solution (2 M in hexane, 4.5 mmol, 5 eq.). The title compound is isolated by preparative RP-HPLC of the crude product.

Yield: 178 mg (34% of theory)

LC-MS (Method 3): Rₜ=3.23 min;

MS (ESIpos): m/z=546 [M+H]⁺;

¹H-NMR (400 MHz, DMSO-d₆): δ=11.71 (s, 1H), 10.14 (s, 1H), 8.83 (s, 1H), 7.86 (d, 1H), 7.32-7.24 (m, 3H), 6.52 (d, 2H), 5.36 (t, 1H), 3.66 (t, 2H), 3.09 (q, 2H), 2.59 (s, 3H), 0.83 (s, 9H), 0.00 (s, 6H).

### Step b): N-{4-[(2-{[tert-Butyl(dimethyl)silyl]oxy}ethyl)(cyano)amino]phenyl}-4-{[(5-chloro-2-thienyl)carbonyl]amino}-2-methylpyrimidine-5-carboxamide

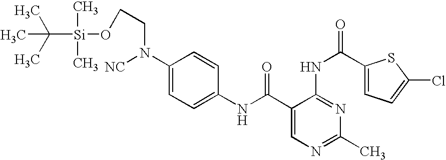

According to General Method 3, 170 mg (0.3 mmol) of N-{4-[(2-{[tert-butyl(dimethyl)silyl]-oxy}ethyl)amino]phenyl}-4-{[(5-chloro-2-thienyl)carbonyl]amino}-2-methylpyrimidine-5-carboxamide are reacted with a total of 125 μl of cyanogen bromide solution (3 M in dichloromethane, 0.4 mmol, 1.2 eq.) in the presence of 78 mg (0.9 mmol, 3 eq.) of sodium bicarbonate. The crude product is reacted without any further purification.

Yield: 156 mg (79% purity, 69% of theory)

LC-MS (Method 2): Rₜ=3.20 min;

MS (ESIpos): m/z=571 [M+H]⁺.

### Step c): 4-{[(5-Chloro-2-thienyl)carbonyl]amino}-N-[4-(2-imino-1,3-oxazolidin-3-yl)-phenyl]-2-methylpyrimidine-5-carboxamide methanesulfonate

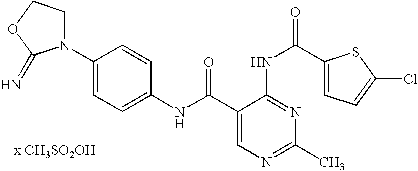

According to General Method 4, 165 mg (0.3 mmol) of N-{4-[(2-{[tert-butyl(dimethyl)silyl]-oxy}ethyl)(cyano)amino]phenyl}-4-{[(5-chloro-2-thienyl)carbonyl]amino}-2-methylpyrimidine-5-carboxamide are reacted with 41 μl (0.6 mmol, 2.2 eq.) of methanesulfonic acid. The title compound is isolated by trituration of the crude product with diethyl ether. It is then purified further by column chromatography on silica gel (mobile phase: dichloromethane/methanol/triethyl-amine 90:10:0.1), and the solid obtained is triturated with 1.0 eq. of methanesulfonic acid in 0.5 ml of acetonitrile. After one hour, the solid is filtered off and washed with diethyl ether.

Yield: 109 mg (65% of theory)

LC-MS (Method 3): Rₜ=1.42 min;

MS (ESIpos): m/z=457 [M+H]⁺ (free base);

¹H-NMR (400 MHz, DMSO-d₆): δ=11.65 (s, 1H), 10.81 (s, 1H), 9.56 (br. s, 1H), 8.85 (s, 1H), 8.84 (br. s, 1H), 8.02 (d, 1H), 7.79 (d, 2H), 7.48 (d, 2H), 7.30 (d, 1H), 4.84 (t, 2H), 4.22 (t, 2H), 2.66 (s, 3H), 2.36 (s, 3H).

### Example 16

### 5-Chloro-N-[4-({[4-(2-imino-1,3-oxazolidin-3-yl)phenyl]amino}carbonyl)-3-thienyl]thiophene-2-carboxamide methanesulfonate

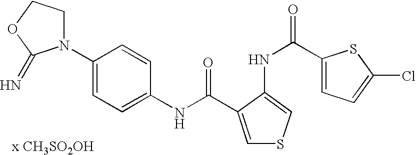

### Step a): N-{4-[({4-[(2-{[tert-Butyl(dimethyl)silyl]oxy}ethyl)amino]phenyl}amino)-carbonyl]-3-thienyl}-5-chlorothiophene-2-carboxamide

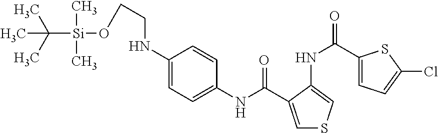

According to General Method 1, 1300 mg (4.5 mmol) of the compound of Example 20A and 1204 mg (4.5 mmol, 1.05 eq.) of the compound of Example 3A are reacted in the presence of 1523 mg of TBTU (4.7 mmol, 1.05 eq.) and 826 μl of N,N-diisopropylethylamine (134 mg, 1.1 eq.). The crude product is purified by column chromatography on silica gel (mobile phase: cyclohexane/ethyl acetate 7:3).

Yield: 1220 mg (50% of theory)

LC-MS (Method 3): Rₜ=3.49 min;

MS (ESIpos): m/z=536 [M+H]⁺;

¹H-NMR (400 MHz, DMSO-d₆): δ=11.90 (s, 1H), 10.07 (s, 1H), 8.50 (d, 1H), 7.91 (d, 1H), 7.47 (d, 1H), 7.32 (d, 2H), 7.22 (d, 1H), 6.56 (d, 2H), 5.43 (t, 1H), 3.67 (t, 2H), 3.11 (q, 2H), 0.83 (s, 9H), 0.00 (s, 6H).

### Step b): N-{4-[({4-[(2-{[tert-Butyl(dimethyl)silyl]oxy}ethyl)(cyano)amino]phenyl}amino)-carbonyl]-3-thienyl}-5-chlorothiophene-2-carboxamide

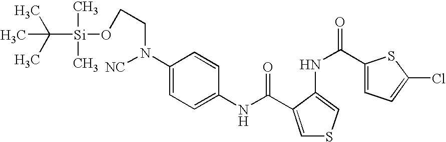

According to General Method 3, 1200 mg (2.2 mmol) of N-{4-[({4-[(2-{[tert-butyl(dimethyl)-silyl]oxy}ethyl)amino]phenyl}amino)carbonyl]-3-thienyl}-5-chlorothiophene-2-carboxamide are reacted with a total of 1492 μl of cyanogen bromide solution (3 M in dichloromethane, 4.5 mmol, 2.0 eq.) in the presence of 564 mg (6.7 mmol, 3 eq.) of sodium bicarbonate. The crude product is reacted without any further purification.

Yield: 230 mg (18% of theory)

LC-MS (Method 3): Rₜ=3.43 min;

MS (ESIpos): m/z=561 [M+H]⁺.

### Step c): 5-Chloro-N-[4-({[4-(2-imino-1,3-oxazolidin-3-yl)phenyl]amino}carbonyl)-3-thienyl]thiophene-2-carboxamide methanesulfonate

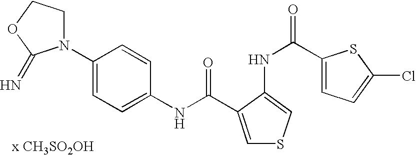

According to General Method 4, 43 mg (0.08 mmol) of N-{4-[({4-[(2-{[tert-butyl(dimethyl)-silyl]oxy}ethyl)(cyano)amino]phenyl}amino)carbonyl]-3-thienyl}-5-chlorothiophene-2-carboxamide are reacted with 11 μl (0.17 mmol, 2.2 eq.) of methanesulfonic acid. The title compound is isolated by trituration of the crude product with diethyl ether.

Yield: 32 mg (77% of theory)

HPLC (Method 9): Rₜ=4.31 min;

MS (ESIpos): m/z=447 [M+H]⁺ (free base);

¹H-NMR (400 MHz, DMSO-d₆): δ=11.53 (s, 1H), 10.63 (s, 1H), 9.59 (br. s, 1H), 8.85 (br. s, 1H), 8.64 (d, 1H), 7.97 (d, 1H), 7.91 (d, 2H), 7.56-7.53 (m, 3H), 7.31 (d, 1H), 4.86 (t, 2H), 4.25 (t, 2H), 2.30 (s, 3H).

### Example 17

### 4-{[(5-Chloro-2-thienyl)carbonyl]amino}-N-[4-(2-imino-1,3-oxazolidin-3-yl)phenyl]isothiazole-3-carboxamide methanesulfonate

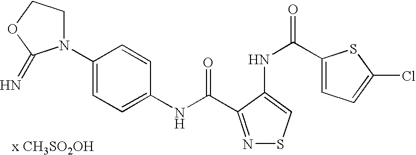

### Step a): N-{4-[(2-{[tert-Butyl(dimethyl)silyl]oxy}ethyl)amino]phenyl}-4-{[(5-chloro-2-thienyl)carbonyl]amino}isothiazole-3-carboxamide

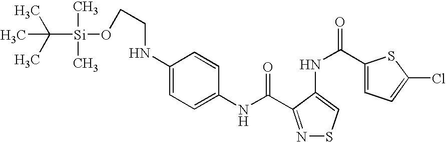

According to General Method 1, 289 mg (1.0 mmol) of the compound of Example 21A and 266 mg (1.0 mmol, 1.0 eq.) of the compound of Example 3A are reacted in the presence of 321 mg of TBTU (1.1 mmol, 1.05 eq.) and 183 μl of N,N-diisopropylethylamine (135 mg, 1.05 eq.). The title compound is isolated by preparative RP-HPLC of the crude product.

Yield: 120 mg (22% of theory)

LC-MS (Method 3): Rₜ=3.60 min;

MS (ESIpos): m/z=537 [M+H]⁺;

¹H-NMR (400 MHz, DMSO-d₆): δ=11.57 (s, 1H), 10.51 (s, 1H), 9.31 (s, 1H), 7.55 (d, 1H), 7.48 (d, 2H), 7.26 (d, 1H), 6.55 (d, 2H), 5.47 (t, 1H), 3.67 (t, 2H), 3.11 (q, 2H), 0.83 (s, 9H), 0.00 (s, 6H).

### Step b): N-{4-[(2-{[tert-Butyl(dimethyl)silyl]oxy}ethyl)(cyano)amino]phenyl}-4-{[(5-chloro-2-thienyl)carbonyl]amino}isothiazole-3-carboxamide

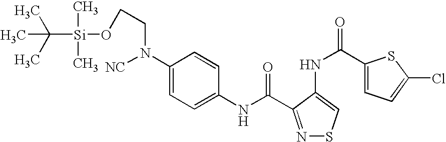

According to General Method 3, 139 mg (0.26 mmol) of N-{4-[(2-{[tert-butyl(dimethyl)-silyl]oxy}ethyl)amino]phenyl}-4-{[(5-chloro-2-thienyl)carbonyl]amino}isothiazole-3-carboxamide are reacted with a total of 104 μl of cyanogen bromide solution (3 M in dichloromethane, 0.31 mmol, 1.2 eq.) in the presence of 65 mg (0.78 mmol, 3 eq.) of sodium bicarbonate. The crude product is reacted further without purification.

Yield: 19 mg (13% of theory).

### Step c): 4-{[(5-Chloro-2-thienyl)carbonyl]amino}-N-[4-(2-imino-1,3-oxazolidin-3-yl)-phenyl]isothiazole-3-carboxamide methanesulfonate

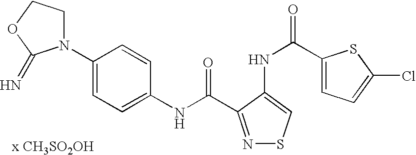

According to General Method 4, 17 mg (0.03 mmol) of N-{4-[(2-{[tert-butyl(dimethyl)-silyl]oxy}ethyl)(cyano)amino]phenyl}-4-{[(5-chloro-2-thienyl)carbonyl]amino}isothiazole-3-carboxamide are reacted with 4 μl (0.06 mmol, 2.2 eq.) of methanesulfonic acid. The title compound is isolated by trituration of the crude product with diethyl ether.

Yield: 4 mg (29% of theory)

LC-MS (Method 1): Rₜ=1.65 min;

MS (ESIpos): m/z=448 [M+H]⁺ (free base);

¹H-NMR (400 MHz, DMSO-d₆): δ=11.30 (s, 1H), 11.17 (s, 1H), 9.60 (br. s, 1H), 9.39 (s, 1H), 8.86 (br. s, 1H), 8.04 (d, 2H), 7.64 (d, 1H), 7.54 (d, 2H), 7.34 (d, 1H), 4.86 (t, 2H), 4.24 (t, 2H), 2.34 (s, 3H).

### Example 18

### 3-{[(5-Chloro-2-thienyl)carbonyl]amino}-N-[4-(2-imino-1,3-oxazolidin-3-yl)phenyl]-1-methyl-1H-pyrazole-4-carboxamide methanesulfonate

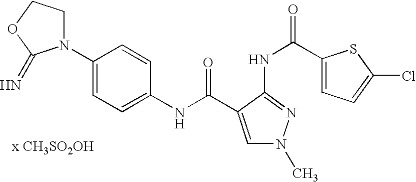

### Step a): N-{4-[(2-{[tert-Butyl(dimethyl)silyl]oxy}ethyl)amino]phenyl}-3-{[(5-chloro-2-thienyl)carbonyl]amino}-1-methyl-1H-pyrazole-4-carboxamide

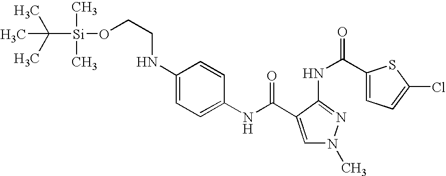

According to General Method 2, 200 mg (0.64 mmol) of the compound of Example 22A and 340 mg (1.28 mmol, 2 eq.) of the compound of Example 3A are reacted in the presence of 1.6 ml of trimethylaluminum solution (2 M in hexane, 3.2 mmol, 5 eq.). The title compound is isolated by preparative RP-HPLC of the crude product.

Yield: 226 mg (66% of theory)

LC-MS (Method 1): Rₜ=2.91 min;

MS (ESIpos): m/z=534 [M+H]⁺.

### Step b): N-{4-[(2-{[tert-Butyl(dimethyl)silyl]oxy}ethyl)(cyano)amino]phenyl}-3-{[(5-chloro-2-thienyl)carbonyl]amino}-1-methyl-1H-pyrazole-4-carboxamide

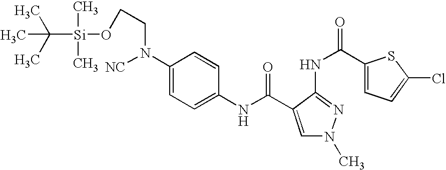

According to General Method 3, 226 mg (0.42 mmol) of N-{4-[(2-{[tert-butyl(dimethyl)-silyl]oxy}ethyl)amino]phenyl}-3-{[(5-chloro-2-thienyl)carbonyl]amino}-1-methyl-1H-pyrazole-4-carboxamide are reacted with a total of 169 μl of cyanogen bromide solution (3 M in dichloromethane, 0.51 mmol, 1.2 eq.) in the presence of 107 mg (1.27 mmol, 3 eq.) of sodium bicarbonate. The title compound is isolated by trituration of the crude product with diethyl ether.

Yield: 201 mg (85% of theory)

LC-MS (Method 3): Rₜ=3.04 min;

MS (ESIpos): m/z=559 [M+H]⁺.

### Step c): 3-{[(5-Chloro-2-thienyl)carbonyl]amino}-N-[4-(2-imino-1,3-oxazolidin-3-yl)-phenyl]-1-methyl-1H-pyrazole-4-carboxamide methanesulfonate

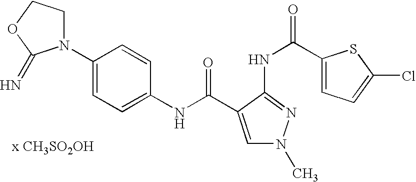

According to General Method 4, 200 mg (0.36 mmol) of N-{4-[(2-{[tert-butyl(dimethyl)-silyl]oxy}ethyl)(cyano)amino]phenyl}-3-{[(5-chloro-2-thienyl)carbonyl]amino}-1-methyl-1H-pyrazole-4-carboxamide are reacted with 49 μl (0.75 mmol, 2.1 eq.) of methanesulfonic acid. The title compound is isolated by filtration of the precipitate formed, washing with acetonitrile and drying under reduced pressure.

Yield: 90 mg (47% of theory)

LC-MS (Method 2): Rₜ=1.41 min;

MS (ESIpos): m/z=445 [M+H]⁺ (free base);

¹H-NMR (400 MHz, DMSO-d₆): δ=11.63 (s, 1H), 10.12 (s, 1H), 9.55 (br. s, 1H), 8.76 (br. s, 1H), 8.33 (s, 1H), 7.87-7.76 (2d, 3H), 7.47 (d, 2H), 7.29 (d, 1H), 4.83 (t, 2H), 4.21 (t, 2H), 3.89 (s, 3H), 2.30 (s, 3H).

### Example 19

### 3-{[(5-Chloro-2-thienyl)carbonyl]amino}-N-[4-(2-imino-1,3-oxazolidin-3-yl)phenyl]thiophene-2-carboxamide methanesulfonate

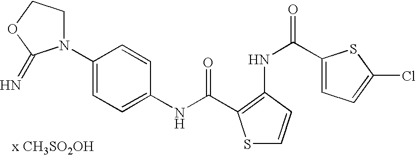

### Step a): N-{4-[(2-{[tert-Butyl(dimethyl)silyl]oxy}ethyl)amino]phenyl}-3-{[(5-chloro-2-thienyl)carbonyl]amino}-thiophene-2-carboxamide

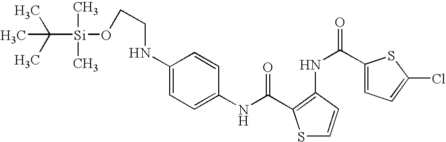

According to General Method 1, 368 mg (1.28 mmol) of the compound of Example 23A and 341 mg (1.28 mmol, 1 eq.) of the compound of Example 3A are reacted in the presence of 431 mg (1.34 mmol, 1.05 eq.) of TBTU and 234 μl (1.34 mmol, 1.05 eq.) of N,N-diisopropylethylamine. The title compound is isolated by preparative RP-HPLC of the crude product.

Yield: 137 mg (18% of theory)

LC-MS (Method 2): Rₜ=3.58 min;

MS (ESIpos): m/z=536 [M+H]⁺;

¹H-NMR (400 MHz, DMSO-d₆): δ=12.09 (s, 1H), 9.83 (s, 1H), 7.96 (d, 1H), 7.85 (d, 1H), 7.59 (d, 1H), 7.46-7.27 (2d, 3H), 6.59 (d, 2H), 5.49 (t, 1H), 3.71 (t, 2H), 3.15 (qd, 2H), 0.88 (s, 9H), 0.05 (s, 6H).

### Step b): N-{4-[(2-{[tert-Butyl(dimethyl)silyl]oxy}ethyl)(cyano)amino]phenyl}-3-{[(5-chloro-2-thienyl)carbonyl]amino}-thiophene-2-carboxamide

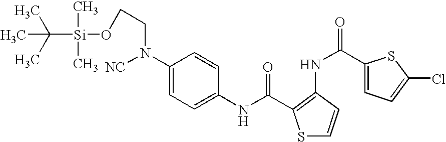

According to General Method 3, 137 mg (0.26 mmol) of N-{4-[(2-{[tert-butyl(dimethyl)-silyl]oxy}ethyl)amino]phenyl}-3-{[(5-chloro-2-thienyl)carbonyl]amino}thiophene-2-carboxamide are reacted with a total of 145 μl of cyanogen bromide solution (3 M in dichloromethane, 0.44 mmol, 1.7 eq.) in the presence of 64 mg (0.77 mmol, 3 eq.) of sodium bicarbonate. The title compound is isolated by trituration of the crude product with diethyl ether.

Yield: 94 mg (66% of theory)

LC-MS (Method 1): Rₜ=3.27 min;

MS (ESIpos): m/z=561 [M+H]⁺.

### Step 2c): 3-{[(5-Chloro-2-thienyl)carbonyl]amino}-N-[4-(2-imino-1,3-oxazolidin-3-yl)-phenyl]thiophene-2-carboxamide methanesulfonate

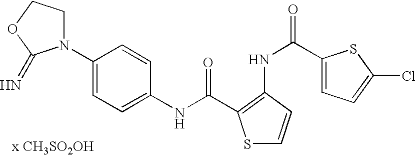

According to General Method 4, 94 mg (0.17 mmol) of N-{4-[(2-{[tert-butyl(dimethyl)silyl]-oxy}ethyl)(cyano)amino]phenyl}-3-{[(5-chloro-2-thienyl)carbonyl]amino}-thiophene-2-carboxamide are reacted with 28 μl (0.44 mmol, 2.6 eq.) of methanesulfonic acid. The title compound is isolated by trituration of the crude product with diethyl ether.

Yield: 70 mg (77% of theory)

LC-MS (Method 1): Rₜ=1.50 min;

MS (ESIpos): m/z=446 [M+H]⁺ (free base);

¹H-NMR (400 MHz, DMSO-d₆): δ=11.77 (s, 1H), 10.39 (s, 1H), 9.59 (br. s, 1H), 8.84 (br. s, 1H), 7.94 (m, 2H), 7.88 (d, 2H), 7.62 (d, 1H), 7.52 (d, 2H), 7.32 (d, 1H), 4.86 (t, 2H), 4.25 (t, 2H), 2.33 (s, 3H).

### Example 20

### 5-Chloro-N-[4-(dimethylamino)-2-({[4-(2-imino-1,3-oxazolidin-3-yl)phenyl]amino}carbonyl)-5-methylphenyl]thiophene-2-carboxamide methanesulfonate

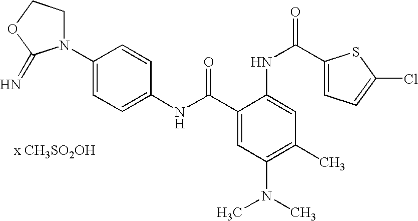

### Step a): N-[2-[({4-[(2-{[tert-Butyl(dimethyl)silyl]oxy}ethyl)amino]phenyl}amino)-carbonyl]-4-(dimethylamino)-5-methylphenyl]-5-chlorothiophene-2-carboxamide

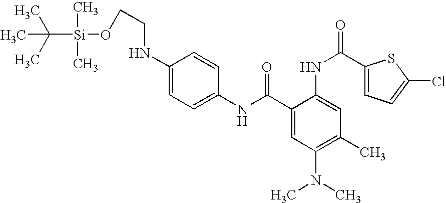

According to General Method 2, 315 mg (0.89 mmol) of the compound of Example 24A and 476 mg (1.79 mmol, 2 eq.) of the compound of Example 3A are reacted in the presence of 2.2 ml of tri-methylaluminum solution (2 M in hexane, 4.46 mmol, 5 eq.). The title compound is isolated by preparative RP-HPLC.

Yield: 340 mg (65% of theory)

LC-MS (Method 3): Rₜ=3.55 min;

MS (ESIpos): m/z=587 [M+H]⁺;

¹H-NMR (400 MHz, DMSO-d₆): δ=11.09 (s, 1H), 10.10 (s, 1H), 8.10 (s, 1H), 7.48 (d, 1H), 7.44 (s, 1H), 7.28 (d, 2H), 7.21 (d, 1H), 6.56 (d, 2H), 5.44 (t, 1H), 3.67 (t, 2H), 3.11 (qd, 2H), 2.65 (s, 6H), 2.28 (s, 3H), 0.83 (s, 9H), 0.00 (s, 6H).

### Step b): N-[2-[({4-[(2-{[tert-Butyl(dimethyl)silyl]oxy}ethyl)(cyano)amino]phenyl}-amino)carbonyl]-4-(dimethylamino)-5-methylphenyl]-5-chlorothiophene-2-carboxamide

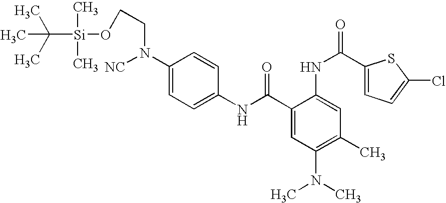

According to General Method 3, 333 mg (0.57 mmol) of N-[2-[({4-[(2-{[tert-butyl-(dimethyl)-silyl]oxy}ethyl)amino]phenyl}amino)carbonyl]-4-(dimethylamino)-5-methylphenyl]-5-chlorothiophene-2-carboxamide are reacted with a total of 227 μl of cyanogen bromide solution (3 M in dichloromethane, 0.68 mmol, 1.2 eq.) in the presence of 143 mg (1.70 mmol, 3 eq.) of sodium bicarbonate. The crude product is used without further purification for the next step.

Yield: 312 mg (90% of theory)

LC-MS (Method 3): Rₜ=3.47 min;

MS (ESIpos): m/z=612 [M+H]⁺;

¹H-NMR (400 MHz, DMSO-d₆): δ=11.45 (s, 1H), 10.43 (s, 1H), 8.01 (s, 1H), 7.71 (d, 2H), 7.60 (d, 1H), 7.47 (s, 1H), 7.27 (d, 1H), 7.22 (d, 2H), 3.89-3.80 (m, 4H), 2.72 (s, 6H), 2.34 (s, 3H), 0.86 (s, 9H), 0.00 (s, 6H).

### Step c): 5-Chloro-N-[4-(dimethylamino)-2-({[4-(2-imino-1,3-oxazolidin-3-yl)phenyl]-amino}carbonyl)-5-methylphenyl]thiophene-2-carboxamide methanesulfonate

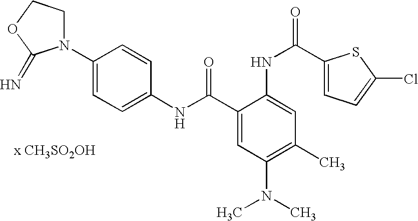

According to General Method 4, 305 mg (0.42 mmol) of N-[2-[({4-[(2-{[tert-butyl(dimethyl)-silyl]oxy}ethyl)(cyano)amino]phenyl}amino)carbonyl]-4-(dimethylamino)-5-methylphenyl]-5-chlorothiophene-2-carboxamide are reacted with 60 μl (0.93 mmol, 2.2 eq.) of methanesulfonic acid. The title compound is isolated by filtration of the precipitate formed, washing with acetonitrile/diethyl ether and drying under reduced pressure.

Yield: 207 mg (82% of theory)

LC-MS (Method 1): Rₜ=1.50 min;

MS (ESIpos): m/z=498 [M+H]⁺ (free base);

¹H-NMR (400 MHz, DMSO-d₆): δ=11.30 (s, 1H), 10.74 (s, 1H), 9.61 (br. s, 1H), 8.86 (br. s, 1H), 7.99 (s, 1H), 7.86 (d, 2H), 7.68 (d, 1H), 7.53 (d, 2H), 7.30 (d, 1H), 4.86 (t, 2H), 4.25 (t, 2H), 3.14 (s, 6H), 2.36 (s, 6H).

### Example 21

### 5-Chloro-N-[4-fluoro-2-({[4-(2-imino-1,3-oxazolidin-3-yl)phenyl]amino}carbonyl)-5-methyl-phenyl]thiophene-2-carboxamide methanesulfonate

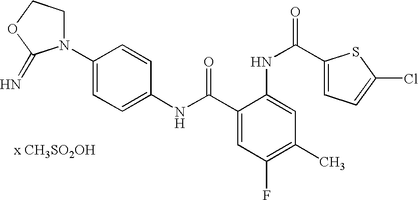

### Step a): N-{2-[({4-[(2-{[tert-Butyl(dimethyl)silyl]oxy}ethyl)amino]phenyl}amino)-carbonyl]-4-fluoro-5-methylphenyl}-5-chlorothiophene-2-carboxamide

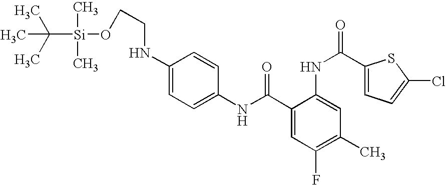

According to General Method 2, 250 mg (0.76 mmol) of the compound of Example 25A and 406 mg (1.53 mmol, 2 eq.) of the compound of Example 3A are reacted in the presence of 1.9 ml of tri-methylaluminum solution (2 M in hexane, 3.81 mmol, 5 eq.). The title compound is isolated by trituration of the crude product with diethyl ether.

Yield: 141 mg (94% purity, 31% of theory)

HPLC (Method 7): Rₜ=5.02 min;

MS (ESIpos): m/z=563 [M+H]⁺;

¹H-NMR (400 MHz, DMSO-d₆): δ=11.98 (s, 1H), 10.14 (s, 1H), 8.22 (d, 1H), 7.72 (d, 1H), 7.53 (d, 1H), 7.32 (d, 2H), 7.23 (d, 1H), 6.55 (d, 2H), 5.44 (t, 1H), 3.67 (t, 2H), 3.10 (qd, 2H), 2.27 (s, 3H), 0.83 (s, 9H), 0.00 (s, 6H).

### Step b): N-{2-[({4-[(2-{[tert-Butyl(dimethyl)silyl]oxy}ethyl)(cyano)amino]phenyl}-amino)carbonyl]-4-fluoro-5-methylphenyl}-5-chlorothiophene-2-carboxamide

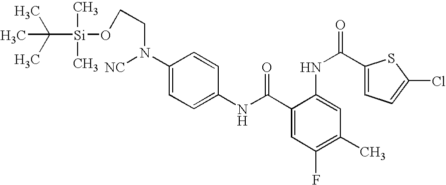

According to General Method 3, 100 mg (0.18 mmol) of N-{2-[({4-[(2-{[tert-butyl(dimethyl)-silyl]oxy}ethyl)amino]phenyl}amino)carbonyl]-4-fluor-5-methylphenyl}-5-chlorothiophene-2-carboxamide are reacted with a total of 71 μl of cyanogen bromide solution (3 M in dichloromethane, 0.21 mmol, 1.2 eq.) in the presence of 45 mg (0.53 mmol, 3 eq.) of sodium bicarbonate. The crude product is used without further purification for the next step.

Yield: 101 mg (90% purity, 87% of theory)

LC-MS (Method 3): Rₜ=3.53 min;

MS (ESIpos): m/z=588 [M+H]⁺;

¹H-NMR (400 MHz, DMSO-d₆): δ=11.53 (s, 1H), 10.47 (s, 1H), 8.13 (d, 1H), 7.78-7.70 (2d, 3H), 7.62 (d, 1H), 7.28 (d, 1H), 7.21 (d, 2H), 3.89-3.80 (m, 4H), 2.32 (s, 3H), 0.84 (s, 9H), 0.00 (s, 6H).

### Step c): 5-Chloro-N-[4-fluoro-2-({[4-(2-imino-1,3-oxazolidin-3-yl)phenyl]amino}carbonyl)-5-methylphenyl]thiophene-2-carboxamide methanesulfonate

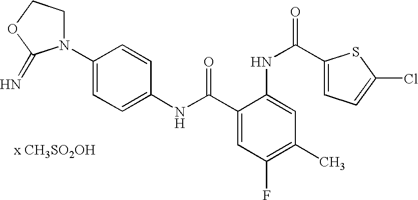

According to General Method 4, 97 mg (0.17 mmol) of N-{2-[({4-[(2-{[tert-butyl(dimethyl)-silyl]oxy}ethyl)(cyano)amino]phenyl}amino)carbonyl]-4-fluoro-5-methylphenyl}-5-chlorothiophene-2-carboxamide are reacted with 24 μl (0.36 mmol, 2.2 eq.) of methanesulfonic acid. The title compound is isolated by filtration of the precipitate formed, washing with acetonitrile/diethyl ether and drying under reduced pressure.

Yield: 71 mg (96% purity, 73% of theory)

LC-MS (Method 3): Rₜ=1.87 min;

MS (ESIpos): m/z=473 [M+H]⁺ (free base);

¹H-NMR (400 MHz, DMSO-d₆): δ=11.27 (s, 1H), 10.66 (s, 1H), 9.58 (br. s, 1H), 8.84 (br. s, 1H), 7.99 (d, 1H), 7.86 (d, 2H), 7.70 (d, 1H), 7.66 (d, 1H), 7.52 (d, 2H), 7.29 (d, 1H), 4.85 (t, 2H), 4.23 (t, 2H), 2.33 (s, 3H), 2.30 (s, 3H).

### Example 22

### 5-Chloro-N-[4-({[4-(2-imino-1,3-oxazolidin-3-yl)phenyl]amino}carbonyl)-1-methyl-1H-pyrazol-3-yl]pyridine-2-carboxamide methanesulfonate

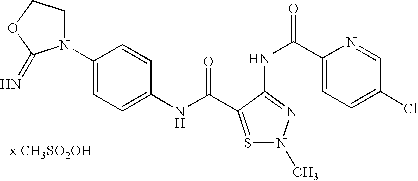

### Step a): N-{4-[({4-[(2-{[tert-Butyl(dimethyl)silyl]oxy}ethyl)amino]phenyl}amino)-carbonyl]-1-methyl-1H-pyrazol-3-yl}-5-chloropyridine-2-carboxamide

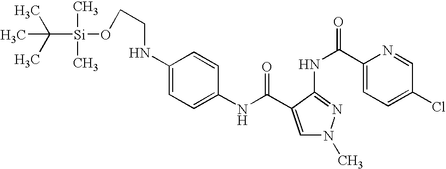

According to General Method 2, 95 mg (0.31 mmol) of the compound of Example 26A and 164 mg (0.62 mmol, 2 eq.) of the compound of Example 3A are reacted in the presence of 769 μl of tri-methylaluminum solution (2 M in hexane, 1.54 mmol, 5 eq.). The title compound is isolated by flash chromatography of the crude product on silica gel (mobile phase: dichloromethane/methanol 50:1→20:1).

Yield: 71 mg (43% of theory)

LC-MS (Method 2): Rₜ=3.01 min;

MS (ESIpos): m/z=529 [M+H]⁺;

¹H-NMR (400 MHz, DMSO-d₆): δ=11.80 (s, 1H), 9.59 (s, 1H), 8.77 (d, 1H), 8.31 (s, 1H), 8.14 (dd, 1H), 8.10 (d, 1H), 7.28 (d, 2H), 6.54 (d, 2H), 5.34 (t, 1H), 3.83 (s, 3H), 3.67 (t, 2H), 3.10 (qd, 2H), 0.83 (s, 9H), 0.00 (s, 6H).

### Step b): N-{4-[({4-[(2-{[tert-Butyl(dimethyl)silyl]oxy}ethyl)(cyano)amino]phenyl}-amino)carbonyl]-1-methyl-1H-pyrazol-3-yl}-5-chloropyridine-2-carboxamide

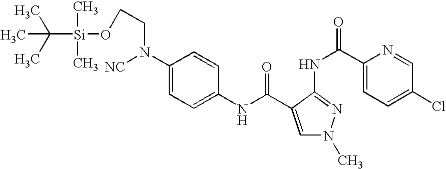

According to General Method 3, 70 mg (0.13 mmol) of N-{4-[({4-[(2-{[tert-butyl(dimethyl)-silyl]oxy}ethyl)amino]phenyl}amino)carbonyl]-1-methyl-1H-pyrazol-3-yl}-5-chloropyridine-2-carboxamide are reacted with a total of 71 μl of cyanogen bromide solution (3 M in dichloromethane, 0.21 mmol, 1.6 eq.) in the presence of 33 mg (0.40 mmol, 3 eq.) of sodium bicarbonate. The title compound is isolated by trituration of the crude product with diisopropyl ether.

Yield: 50 mg (68% of theory)

LC-MS (Method 1): Rₜ=2.70 min;

MS (ESIpos): m/z=554 [M+H]⁺;

¹H-NMR (400 MHz, DMSO-d₆): δ=11.75 (s, 1H), 9.99 (s, 1H), 8.85 (s, 1H), 8.43 (s, 1H), 8.24-8.15 (m, 2H), 7.72 (d, 2H), 7.22 (d, 2H), 3.91 (s, 3H), 3.88-3.80 (m, 4H), 0.86 (s, 9H), 0.00 (s, 6H).

### Step c): 5-Chloro-N-[4-({[4-(2-imino-1,3-oxazolidin-3-yl)phenyl]amino}carbonyl)-1-methyl-1H-pyrazol-3-yl]pyridine-2-carboxamide methanesulfonate

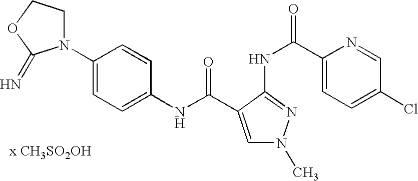

According to General Method 4, 46 mg (0.08 mmol) of N-{4-[({4-[(2-{[tert-butyl(dimethyl)-silyl]oxy}ethyl)(cyano)amino]phenyl}amino)carbonyl]-1-methyl-1H-pyrazol-3-yl}-5-chlor-pyridine-2-carboxamide are reacted with 11 μl (0.17 mmol, 2.1 eq.) of methanesulfonic acid. The title compound is isolated by flash chromatography on silica gel (mobile phase: dichloromethane/methanol 9:1→4:1).

Yield: 13.2 mg (27% of theory)

LC-MS (Method 1): Rₜ=1.13 min;

MS (ESIpos): m/z=440 [M+H]⁺ (free base);

¹H-NMR (400 MHz, DMSO-d₆): δ=11.69 (s, 1H), 10.18 (s, 1H), 9.56 (br. s, 1H), 8.82 (s, 1H), 8.79 (br. s, 1H), 8.47 (s, 1H), 8.21 (dd, 1H), 8.17 (d, 1H), 7.87 (d, 2H), 7.50 (d, 2H), 4.86 (t, 2H), 4.24 (t, 2H), 3.91 (s, 3H), 2.30 (s, 3H).

### Example 23

### 5-Chloro-N-[4-cyano-2-({[4-(2-imino-1,3-oxazolidin-3-yl)phenyl]amino}carbonyl)-5-methyl-phenyl]thiophene-2-carboxamide methanesulfonate

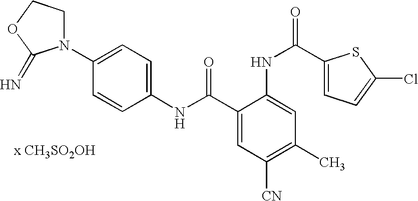

### Step a): N-{2-[({4-[(2-{[tert-Butyl(dimethyl)silyl]oxy}ethyl)amino]phenyl}amino)-carbonyl]-4-cyano-5-methylphenyl}-5-chlorothiophene-2-carboxamide

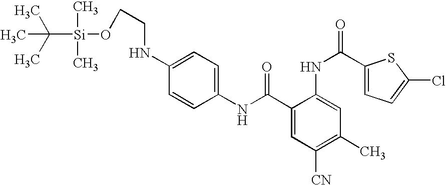

According to General Method 2, 124 mg (0.4 mmol) of the compound of Example 27A and 197 mg (0.7 mmol, 2.0 eq.) of the compound of Example 5A are reacted in the presence of 0.9 ml of trimethylaluminum solution (2 M in hexane, 1.9 mmol, 5.0 eq.). The title compound is obtained by purification by RP-HPLC.

Yield: 77 mg (34% of theory)

LC-MS (Method 3): Rₜ=3.61 min;

MS (ESIpos): m/z=569 [M+H]⁺;

¹H-NMR (400 MHz, DMSO-d₆): δ=12.53 (s, 1H), 10.31 (s, 1H), 8.48 (s, 1H), 8.35 (s, 1H), 7.53 (d, 1H), 7.34 (d, 2H), 7.26 (d, 1H), 6.56 (d, 2H), 5.49 (br. s, 1H), 3.67 (t, 2H), 3.14-3.08 (m, 2H), 2.52 (s, 3H), 0.83 (s, 9H), 0.02 (s, 6H).

### Step b): N-{2-[({4-[(2-{[tert-Butyl(dimethyl)silyl]oxy}ethyl)(cyano)amino]phenyl}amino)-carbonyl]-4-cyano-5-methylphenyl}-5-chlorothiophene-2-carboxamide

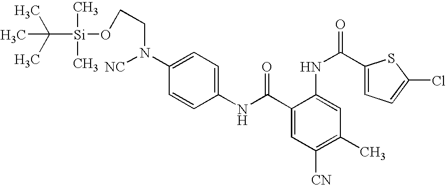

According to General Method 3, 22 mg (0.04 mmol) of N-{2-[({4-[(2-{[tert-butyl(dimethyl)-silyl]oxy}ethyl)amino]phenyl}amino)carbonyl]-4-cyano-5-methylphenyl}-5-chlorothiophene-2-carboxamide are reacted with 15 μl of cyanogen bromide solution (3 M in dichloromethane, 0.05 mmol, 1.2 eq.) in the presence of 10 mg (0.12 mmol, 3.0 eq.) of sodium bicarbonate. After work-up as described in General Method 3, the title compound is directly reacted further.

Yield: 16 mg (68% of theory)

LC-MS (Method 1): Rₜ=3.35 min;

MS (ESIpos): m/z=594 [M+H]⁺;

¹H-NMR (400 MHz, DMSO-d₆): δ=12.11 (s, 1H), 10.63 (s, 1H), 8.44 (s, 1H), 8.40 (s, 1H), 7.74 (d, 2H), 7.63 (d, 1H), 7.31 (d, 1H), 7.23 (d, 2H), 3.90-3.78 (m, 4H), 2.55 (s, 3H), 0.84 (s, 9H), 0.02 (s, 6H).

### Step c): 5-Chloro-N-[4-cyano-2-({[4-(2-imino-1,3-oxazolidin-3-yl)phenyl]amino}-carbonyl)-5-methylphenyl]thiophene-2-carboxamide methanesulfonate

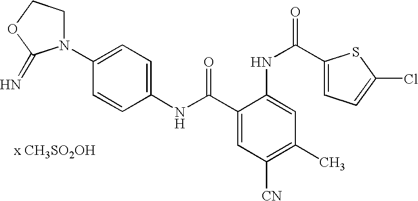

According to General Method 4, 14 mg (0.02 mmol) of N-{2-[({4-[(2-{[tert-butyl(dimethyl)-silyl]oxy}ethyl)(cyano)amino]phenyl}amino)carbonyl]-4-cyano-5-methylphenyl}-5-chlorothiophene-2-carboxamide are reacted with 3 μl (0.04 mmol, 2.2 eq.) of methanesulfonic acid. The title compound is isolated by trituration with diethyl ether and subsequent filtration.

Yield: 11 mg (95% of theory)

HPLC (Method 7): Rₜ=4.70 min;

MS (ESIpos): m/z=480 [M+H]⁺ (free base);

¹H-NMR (400 MHz, DMSO-d₆): δ=11.86 (s, 1H), 10.80 (s, 1H), 9.59 (br. s, 1H), 8.86 (br. s, 1H), 8.36 (s, 1H), 8.34 (s, 1H), 7.86 (d, 2H), 7.65 (d, 1H), 7.54 (d, 2H), 7.33 (d, 1H), 4.86 (t, 2H), 4.25 (t, 2H), 2.52 (s, 3H), 2.33 (s, 3H).

### Example 24

### 4-{[(5-Chloro-2-thienyl)carbonyl]amino}-N-[4-(2-imino-1,3-oxazolidin-3-yl)phenyl]-2-(methyl-thio)-1,3-thiazole-5-carboxamide methanesulfonate

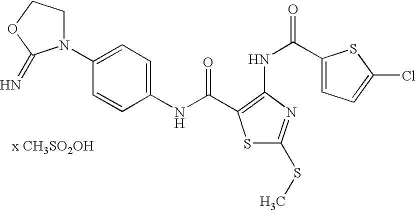

### Step a): N-{4-[(2-{[tert-Butyl(dimethyl)silyl]oxy}ethyl)amino]phenyl}4-{[(5-chloro-2-thienyl)carbonyl]amino}-2-(methylthio)-1,3-thiazole-5-carboxamide

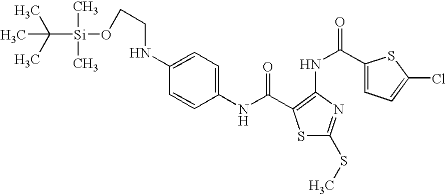

According to General Method 2, 3.0 g (6.2 mmol) of the compound of Example 28A and 3.3 g (12.4 mmol, 2.0 eq.) of the compound of Example 5A are reacted in the presence of 15.5 ml of tri-methylaluminum solution (2 M in hexane, 31.0 mmol, 5.0 eq.). The title compound is obtained by purification by RP-HPLC.

Yield: 1.7 g (46% of theory)

LC-MS (Method 3): Rₜ=3.38 min;

MS (ESIpos): m/z=583 [M+H]⁺;

¹H-NMR (400 MHz, DMSO-d₆): δ=11.22 (s, 1H), 9.68 (s, 1H), 7.81 (d, 1H), 7.25-7.21 (m, 3H), 6.51 (d, 2H), 5.40 (br. s, 1H), 3.66 (t, 2H), 3.11-3.08 (m, 2H), 2.51 (s, 3H), 0.83 (s, 9H), 0.02 (s, 6H).

### Step b): N-{4-[(2-{[tert-Butyl(dimethyl)silyl]oxy}ethyl)(cyano)amino]phenyl}-4-{[(5-chloro-2-thienyl)carbonyl]amino}-2-(methylthio)-1,3-thiazole-5-carboxamide

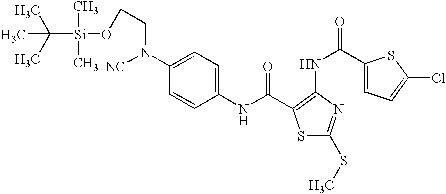

According to General Method 3, 200 mg (0.34 mmol) of N-{4-[(2-{[tert-butyl(dimethyl)-silyl]oxy}ethyl)amino]phenyl}-4-{[(5-chloro-2-thienyl)carbonyl]amino}-2-(methylthio)-1,3-thiazole-5-carboxamide are reacted with 137 μl of cyanogen bromide solution (3 M in dichloromethane, 0.41 mmol, 1.2 eq.) in the presence of 86 mg (1.03 mmol, 3.0 eq.) of sodium bicarbonate. After work-up as described in General Method 3, the title compound is directly reacted further.

Yield: 206 mg (94% of theory)

LC-MS (Method 1): Rₜ=3.14 min;

MS (ESIpos): m/z=608 [M+H]⁺;

¹H-NMR (400 MHz, DMSO-d₆): δ=11.27 (s, 1H), 10.16 (br. s, 1H), 7.90 (s, 1H), 7.62 (d, 2H), 7.30 (d, 1H), 7.17 (d, 2H), 3.85-3.81 (m, 4H), 2.57 (s, 3H), 0.86 (s, 9H), 0.03 (s, 6H).

### Step c): 4-{[(5-Chloro-2-thienyl)carbonyl]amino}-N-[4-(2-imino-1,3-oxazolidin-3-yl)-phenyl]-2-(methylthio)-1,3-thiazole-5-carboxamide methanesulfonate

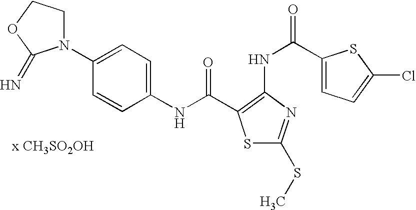

According to General Method 4, 195 mg (0.31 mmol) of N-{4-[(2-{[tert-butyl(dimethyl)-silyl]oxy}ethyl)(cyano)amino]phenyl}-4-{[(5-chloro-2-thienyl)carbonyl]amino}-2-(methylthio)-1,3-thiazole-5-carboxamide are reacted with 43 μl (0.67 mmol, 2.2 eq.) of methanesulfonic acid.

The title compound is obtained by trituration with diethyl ether and subsequent filtration.

Yield: 146 mg (81% of theory)

LC-MS (Method 1): Rₜ=1.43 min;

MS (ESIpos): m/z=494 [M+H]⁺ (free base);

¹H-NMR (400 MHz, DMSO-d₆): δ=11.30 (s, 1H), 10.35 (s, 1H), 9.55 (br. s, 1H), 8.80 (br. s, 1H), 7.92 (s, 1H), 7.74 (d, 2H), 7.46 (d, 2H), 7.29 (d, 1H), 4.84 (t, 2H), 4.21 (t, 2H), 2.75 (s, 3H), 2.37 (s, 3H).

### Example 25

### 4-{[(5-Chloro-2-thienyl)carbonyl]amino}-N-[4-(2-imino-1,3-oxazolidin-3-yl)phenyl]-2-(methyl-sulfonyl)-1,3-thiazole-5-carboxamide methanesulfonate

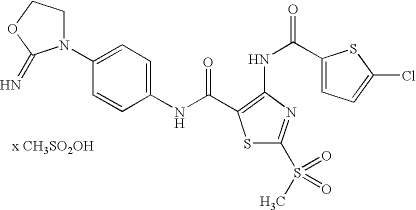

34 mg (0.06 mmol) of the compound of Example 24 are dissolved in 1 ml of concentrated acetic acid, and 1.0 ml of 30% strength aqueous hydrogen peroxide solution are added. The mixture is stirred at room temperature for 22 hours, 15 ml of THF are then added and the mixture is dried over sodium sulfate, filtered and concentrated under reduced pressure.

Yield: 27 mg (71% of theory)

LC-MS (Method 1): Rₜ=1.30 min;

MS (ESIpos): m/z=526 [M+H]⁺ (free base);

¹H-NMR (400 MHz, DMSO-d₆): δ=11.77 (s, 1H), 10.82 (s, 1H), 9.57 (br. s, 1H), 8.81 (br. s, 1H), 8.00 (s, 1H), 7.75 (d, 2H), 7.49 (d, 2H), 7.30 (d, 1H), 4.82 (t, 2H), 4.23 (t, 2H), 3.56 (s, 3H), 2.37 (s, 3H).

### Example 26

### 4-{[(5-Chloro-2-thienyl)carbonyl]amino}-N-[4-(2-imino-1,3-oxazolidin-3-yl)phenyl]-2-(methyl-sulfonyl)-1,3-thiazole-5-carboxamide

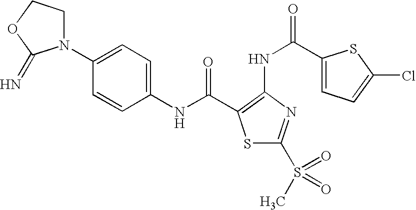

100 mg (0.2 mmol) of the compound of Example 24 are dissolved in 3 ml of concentrated acetic acid, and 3.0 ml of 30% strength hydrogen peroxide solution are added. The mixture is stirred at room temperature for 22 hours, 15 ml of THF are then added and the mixture is dried over sodium sulfate, filtered and concentrated under reduced pressure. The residue is purified by RP-HPLC.

Yield: 28 mg (27% of theory)

LC-MS (Method 3): Rₜ=1.53 min;

MS (ESIpos): m/z=526 [M+H]⁺;

¹H-NMR (400 MHz, DMSO-d₆): δ=11.30 (s, 1H), 9.30 (s, 1H), 7.82 (s, 1H), 7.74 (d, 2H), 7.52 (d, 2H), 7.24 (d, 1H), 4.82-4.76 (m, 2H), 4.23-4.19 (m, 2H), 3.52 (s, 3H).

### Example 27

### 5-Chloro-N-(2-{[4-(2-imino-1,3-oxazolidin-3-yl)phenyl]carbamoyl}-5-methylphenyl)-pyridine-2-carboxamide methanesulfonate

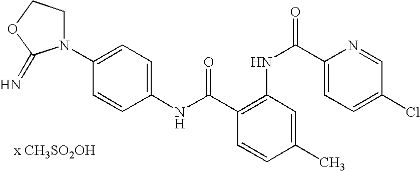

### Step a): N-[2-({4-[(2-{[tert-Butyl(dimethyl)silyl]oxy}ethyl)amino]phenyl}carbamoyl)-5-methylphenyl]-5-chloropyridine-2-carboxamide

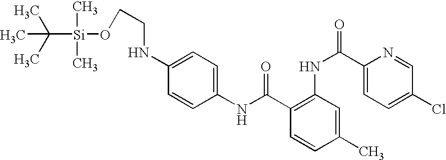

According to General Method 2, 62 mg (0.2 mmol) of the compound of Example 29A and 108 mg (0.4 mmol, 2.0 eq.) of the compound of Example 3A are reacted in the presence of 0.4 ml of trimethylaluminum solution (2 M in toluene, 0.8 mmol, 4.0 eq.). The title compound is obtained by purification by RP-HPLC.

Yield: 20 mg (18% of theory)

LC-MS (Method 1): Rₜ=3.25 min;

MS (ESIpos): m/z=539 [M+H]⁺;

¹H-NMR (400 MHz, DMSO-d₆): δ=12.61 (s, 1H), 10.10 (s, 1H), 8.80 (s, 1H), 8.54 (s, 1H), 8.20-8.15 (m, 2H), 7.78 (d, 1H), 7.37 (d, 2H), 7.09 (d, 1H), 6.60 (d, 2H), 5.44 (t, 1H), 3.72 (t, 2H), 3.17-3.13 (m, 2H), 2.41 (s, 3H), 0.88 (s, 9H), 0.05 (s, 6H).

### Step b): N-[2-({4-[(2-{[tert-Butyl(dimethyl)silyl]oxy}ethyl)(cyano)amino]phenyl}1-carbamoyl)-5-methylphenyl]-5-chloropyridine-2-carboxamide

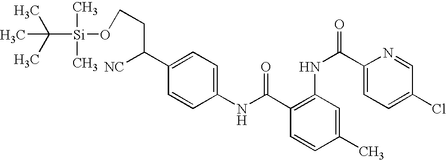

According to General Method 3, 20 mg (0.04 mmol) of N-[2-({4-[(2-{[tert-butyl(dimethyl)-silyl]oxy}ethyl)amino]phenyl}carbamoyl)-5-methylphenyl]-5-chloropyridine-2-carboxamide are reacted with 15 μl of cyanogen bromide solution (3 M in dichloromethane, 0.05 mmol, 1.2 eq.) in the presence of 9 mg (0.11 mmol, 3.0 eq.) of sodium bicarbonate. After work-up as described in General Method 3, the title compound is obtained by purification by RP-HPLC.

Yield: 10 mg (48% of theory)

LC-MS (Method 1): Rₜ=3.17 min;

MS (ESIpos): m/z=564 [M+H]⁺.

### Step c): 5-Chloro-N-(2-{[4-(2-imino-1,3-oxazolidin-3-yl)phenyl]carbamoyl}-5-methyl-phenyl)pyridine-2-carboxamide methanesulfonate

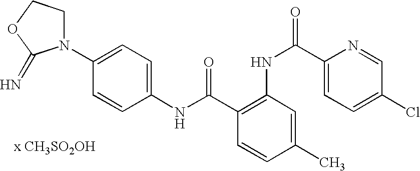

According to General Method 4, 10 mg (0.02 mmol) of N-[2-({4-[(2-{[tert-butyl(dimethyl)-silyl]oxy}ethyl)(cyano)amino]phenyl}carbamoyl)-5-methylphenyl]-5-chloropyridine-2-carboxamide are reacted with 3 μl (0.04 mmol, 2.2 eq.) of methanesulfonic acid. The title compound is obtained by trituration with diethyl ether and subsequent filtration.

Yield: 3 mg (31% of theory)

LC-MS (Method 2): Rₜ=1.87 min;

MS (ESIpos): m/z=450 [M+H]⁺ (free base);

¹H-NMR (400 MHz, DMSO-d₆): δ=12.37 (s, 1H), 10.70 (s, 1H), 9.57 (br. s, 1H), 8.84 (br. s, 1H), 8.79 (s, 1H), 8.53 (s, 1H), 8.23-8.17 (m, 2H), 7.89 (d, 2H), 7.82 (d, 1H), 7.54 (d, 2H), 7.16 (d, 1H), 4.86 (t, 2H), 4.24 (t, 2H), 2.43 (s, 3H), 2.29 (s, 3H).

### B. EVALUATION OF THE PHARMACOLOGICAL ACTIVITY

The compounds according to the invention act in particular as selective inhibitors of blood coagulation factor Xa and do not, or only at significantly higher concentrations, inhibit other serine proteases, such as plasmin or trypsin.

Inhibitors of blood coagulation factor Xa are referred to as being “selective” if the IC₅₀ values for factor Xa inhibition are smaller by a factor of at least 100 compared with the IC₅₀ values for the inhibition of other serine proteases, in particular plasmin and trypsin, where, with a view to the test methods for selectivity, reference is made to the test methods described below of Examples B.a.1) and B.a.2).

The advantageous pharmacological properties of the compounds according to the invention can be determined by the following methods:

### a) Test Descriptions (In Vitro)

### a.1) Determination of the Factor Xa Inhibition:

The enzymatic activity of human factor Xa (FXa) is measured using the conversion of a chromogenic substrate specific for FXa. Factor Xa cleaves p-nitroaniline from the chromogenic substrate. The determinations are carried out in microtiter plates as follows:

The test substances, in various concentrations, are dissolved in DMSO and incubated for 10 minutes at 25° C. with human FXa (0.5 nmol/l dissolved in 50 mmol/l of Tris buffer [C,C,C-tris(hydroxymethyl)aminomethane], 150 mmol/l of NaCl, 0.1% BSA [bovine serum albumin], pH=8.3). Pure DMSO is used as control. The chromogenic substrate (150 μmol/l Pefachrome® FXa from Pentapharm) is then added. After an incubation time of 20 minutes at 25° C., the extinction at 405 nm is determined. The extinctions of the test mixtures containing the test substance are compared with the control mixtures without test substance, and the IC₅₀ values are calculated from these data.

Representative activity data from this test are listed in Table 1 below:

TABLE 1

| Example No. | IC₅₀ [nM] |
|---|---|
| 3 | 1.1 |
| 11 | 6.3 |
| 16 | 2.2 |
| 20 | 0.66 |
| 24 | 0.91 |

### a.2) Determination of the Selectivity:

To assess selective FXa inhibition, the test substances are examined for their inhibition of other human serine proteases such as trypsin and plasmin. To determine the enzymatic activity of trypsin (500 mU/ml) and plasmin (3.2 mol/l), these enzymes are dissolved in Tris buffer (100 mmol/l, 20 mmol/l CaCl₂, pH=8.0) and incubated with test substance or solvent for 10 minutes. The enzymatic reaction is then started by adding the corresponding specific chromogenic substrates (Chromozym Trypsin® and Chromozym Plasmin®; from Roche Diagnostics) and the extinction at 405 nm is determined after 20 minutes. All determinations are carried out at 37° C. The extinctions of the test mixtures containing test substance are compared with the control samples without test substance, and the IC₅₀ values are calculated from these data.

### a.3) Determination of the Anticoagulant Action:

The anticoagulant action of the test substances is determined in vitro in human and rabbit plasma. To this end, blood is drawn off in a mixing ratio of sodium citrate/blood of 1:9 using a 0.11 molar sodium citrate solution as receiver. Immediately after the blood has been drawn off, it is mixed thoroughly and centrifuged at about 2500 g for 10 minutes. The supernatant is pipetted off. The prothrombin time (PT, synonyms: thromboplastin time, quick test) is determined in the presence of varying concentrations of test substance or the corresponding solvent using a commercial test kit (Hemoliance® RecombiPlastin, from Instrumentation Laboratory). The test compounds are incubated with the plasma at 37° C. for 3 minutes. Coagulation is then started by addition of thromboplastin, and the time when coagulation occurs is determined. Concentration of test substance which effects a doubling of the prothrombin time is determined.

### b) Determination of the Antithrombotic Activity (In Vivo)

### b. 1) Arteriovenous Shunt Model (Rabbit):

Fasting rabbits (strain: Esd: NZW) are anaesthetized by intramuscular administration of Rompun/Ketavet solution (5 mg/kg and 40 mg/kg, respectively). Thrombus formation is initiated in an arteriovenous shunt in accordance with the method described by C. N. Berry et al. [Semin. Thromb. Hemost. 1996, 22, 233-241]. To this end, the left jugular vein and the right carotid artery are exposed. The two vessels are connected by an extracorporeal shunt using a vein catheter of a length of 10 cm. In the middle, this catheter is attached to a further polyethylene tube (PE 160, Becton Dickenson) of a length of 4 cm which contains a roughened nylon thread which has been arranged to form a loop, to form a thrombogenic surface. The extracorporeal circulation is maintained for 15 minutes. The shunt is then removed and the nylon thread with the thrombus is weighed immediately. The weight of the nylon thread on its own was determined before the experiment was started. Before extracorporeal circulation is set up, the test substances are administered either intravenously via an ear vein or orally using a pharyngeal tube.

### c) Determination of the Solubility

### Reagents Required:

PBS buffer pH 7.4: 90.00 g of NaCl p.a. (for example from Merck, Art. No. 1.06404.1000), 13.61 g of KH₂PO₄ p.a. (for example from Merck, Art. No. 1.04873.1000) and 83.35 g of 1 N NaOH (for example from Bernd Kraft GmbH, Art. No. 01030.4000) are weighed out into a 1 liter measuring flask, the flask is filled with water and the mixture is stirred for about 1 hour;
acetate buffer pH 4.6: 5.4 g of sodium acetate×3H₂O p.a. (for example from Merck, Art. No. 1.06267.0500) are weighed out into a 100 ml measuring flask and dissolved in 50 ml of water, 2.4 g of glacial acetic acid are added, the flask is filled to 100 ml with water, the pH is checked and, if required, adjusted to pH 4.6;
dimethyl sulfoxide (for example from Baker, Art. No. 7157.2500);
distilled water.

### Preparation of the Calibration Solutions:

Preparation of the starting solution for calibration solutions (stock solution): about 0.5 mg of the test substance are weighed out accurately into a 2 ml Eppendorf Safe-Lock tube (from Eppendorf, Art. No. 0030 120.094), DMSO is added to a concentration of 600 μg/ml (for example 0.5 mg of substance+833 μl of DMSO) and the mixture is vortexed until complete solution is achieved.

Calibration solution 1 (20 μg/ml): 1000 μl of DMSO are added to 34.4 μl of the stock solution, and the mixture is homogenised.

Calibration solution 2 (2.5 μg/ml): 700 μl of DMSO are added to 100 μl of calibration solution 1, and the mixture is homogenised.

### Preparation of the Sample Solutions:

Sample solution for solubilities of up to 10 μl in PBS buffer pH 7.4: about 5 mg of the test substance are weighed out accurately into a 2 ml Eppendorf Safe-Lock tube (from Eppendorf, Art.-Nr. 0030 120.094), and PBS buffer pH 7.4 is added to a concentration of 5 g/l (for example 5 mg of substance+500 μl of PBS buffer pH 7.4).

Sample solution for solubilities of up to 10 μl in acetate buffer pH 4.6: about 5 mg of the test substance are weighed out accurately into a 2 ml Eppendorf Safe-Lock tube (from Eppendorf, Art.-Nr. 0030 120.094), and acetate buffer pH 4.6 is added to a concentration of 5 g/l (for example 5 mg of substance+500 μl of acetate buffer pH 4.6).

Sample solution for solubilities of up to 10 μl in water: about 5 mg of the test substance are weighed out accurately into a 2 ml Eppendorf Safe-Lock tube (from Eppendorf, Art.-Nr. 0030 120.094), and water is added to a concentration of 5 g/l (for example 5 mg of substance+500 μl of water).

### Practice:

For 24 hours, the sample solutions prepared in this manner are shaken at 1400 rpm using a temperature-controlled shaker (for example the Eppendorf Thermomixer comfort Art. No. 5355 000.011 with interchangeable block Art. No. 5362.000.019) at 20° C. In each case 180 μl are removed from these solutions and transferred into Beckman Polyallomer centrifuge tubes (Art. No. 343621). These solutions are centrifuged at about 223.000×g (for example Beckman Optima L-90K Ultracentrifuge with type 42.2 Ti rotor at 42 000 rpm) for 1 hour. From each sample solution, 100 μl of the supernatant are removed and diluted 1:5, 1:100 and 1:1000 with the respective solvent used (water, PBS buffer 7.4 or acetate buffer pH 4.6). From each dilution, a sample is filled into a suitable vessel for HPLC analysis.

### Analysis:

The samples are analyzed by RP-HPLC. Quantification is carried out using a two-point calibration curve of the test compound in DMSO. The solubility is expressed in mg/l. Analysis sequence: 1) calibration solution 2.5 mg/ml; 2) calibration solution 20 μg/ml; 3) sample solution 1:5; 4) sample solution 1:100; 5) sample solution 1:1000.

### HPLC Method for Acids:

Agilent 1100 with DAD (G13 15A), quat. pump (G1311A), autosampler CTC HTS PAL, degaser (G1322A) and column thermostat (G1316A); column: Phenomenex Gemini C18, 50 mm×2 mm, 5μ; temperature: 40° C.; mobile phase A: water/phosphoric acid pH 2; mobile phase B: acetonitrile; flow rate: 0.7 ml/min; gradient: 0-0.5 min 85% A, 15% B; ramp: 0.5-3 min 10% A, 90% B; 3-3.5 min 10% A, 90% B; ramp: 3.54 min 85% A, 15% B; 4-5 min 85% A, 15% B.

### HPLC Method for Bases:

Agilent 1100 with DAD (G1315A), quat. pump (G1311A), autosampler CTC HTS PAL, degaser (G1322A) and column thermostat (G1316A); column: VDSoptilab Kromasil 100 C18, 60 mm×2.1 mm, 3.5μ; temperature: 30° C.; mobile phase A: water+5 ml perchloric acid/l; mobile phase B: acetonitrile; flow rate: 0.75 ml/min; gradient: 0-0.5 min 98% A, 2% B; ramp: 0.5-4.5 min 10% A, 90% B; 4.5-6 min 10% A, 90% B; ramp: 6.5-6.7 min 98% A, 2% B; 6.7-7.5 min 98% A, 2% B. C. WORKING EXAMPLES OF PHARMACEUTICAL COMPOSITIONS

The compounds according to the invention can be converted into pharmaceutical preparations in the following ways:

### Tablet:

### Composition:

100 mg of the compound according to the invention, 50 mg of lactose (monohydrate), 50 mg of maize starch (native), 10 mg of polyvinylpyrrolidone (PVP 25) (from BASF, Ludwigshafen, Germany) and 2 mg of magnesium stearate.

Tablet weight 212 mg. Diameter 8 mm, radius of curvature 12 mm.

### Production:

The mixture of the compound according to the invention, lactose and starch is granulated with a 5% strength solution (m/m) of the PVP in water. The granules are dried and then mixed with the magnesium stearate for 5 minutes. This mixture is compressed using a conventional tablet press (see above for the dimensions of the tablet). A compressive force of 15 kN is used as a guideline for the compression.

Suspension which can be Administered Orally:

### Composition:

1000 mg of the compound according to the invention, 1000 mg of ethanol (96%), 400 mg of Rhodigel® (xanthan gum from FMC, Pennsylvania, USA) and 99 g of water. 10 ml of oral suspension correspond to a single dose of 100 mg of the compound according to the invention.

### Production:

The Rhodigel is suspended in ethanol, and the compound according to the invention is added to the suspension. The water is added while stirring. The mixture is stirred for about 6 h until the swelling of the Rhodigel is complete.

Solution which can be Administered Orally:

### Composition:

500 mg of the compound according to the invention, 2.5 g of polysorbate and 97 g of polyethylene glycol 400.20 g of oral solution correspond to a single dose of 100 mg of the compound according to the invention.

### Production:

The compound according to the invention is suspended in the mixture of polyethylene glycol and polysorbate with stirring. Stirring is continued until the compound according to the invention has dissolved completely.

i.v. solution:

The compound according to the invention is, at a concentration below saturation solubility, dissolved in a physiologically acceptable solvent (for example isotonic saline, glucose solution 5% and/or PEG 400 solution 30%). The solution is subjected to sterile filtration and filled into sterile and pyrogen-free injection containers.

1. A compound of the formula (I) 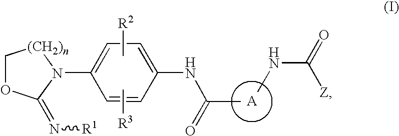
in which
n represents the number 1, 2 or 3,
R¹ represents hydrogen, hydroxyl, (C₁-C₄)-alkyl, (C₁-C₄)-alkanoyl or cyano,
R² and R³ are identical or different and independently of one another represent hydrogen, fluorine, chlorine, cyano, (C₁-C₄)-alkyl, cyclopropyl, trifluoromethyl, hydroxyl, (C₁-C₄)-alkoxy, trifluoromethoxy, amino, mono- or di-(C₁-C₄)-alkylamino,
A represents a phenylene ring or a 5- or 6-membered heteroarylene ring where the two groupings —CO—NH-phenyl and —NH—CO-Z are located at adjacent ring atoms of the phenylene or heteroarylene ring and where phenylene and heteroarylene may additionally be substituted by substituents selected from the group consisting of fluorine, chlorine, cyano, (C₁-C₆)-alkyl, (C₃-C₇)-cycloalkyl, trifluoromethyl, hydroxyl, (C₁-C₆)-alkoxy, trifluoromethoxy, amino, mono- and di-(C₁-C₆)-alkylamino, (C₃-C₇)-cycloalkylamino, (C₁-C₆)-alkanoylamino, (C₁-C₆)-alkoxycarbonylamino, (C₁-C₆)-alkylthio, (C₁-C₆)-alkylsulfonyl, hydroxycarbonyl, (C₁-C₆)-alkoxycarbonyl, aminocarbonyl, mono- and di-(C₁-C₆)-alkylaminocarbonyl,
where (C₁-C₆)-alkyl, (C₁-C₆)-alkoxy, mono- and di-(C₁-C₆)-alkylamino for their part may each be substituted by hydroxyl, (C₁-C₄)-alkoxy, amino, mono- or di-(C₁-C₄)-alkylamino or (C₃-C₅)-cycloalkylamino,
and
Z represents phenyl, pyridyl, pyrimidinyl, pyrazinyl or thienyl which may in each case be mono- or disubstituted by identical or different substituents selected from the group consisting of fluorine, chlorine, cyano, methoxy, (C₁-C₄)-alkyl (which for its part may be substituted by amino), ethynyl and amino,
or a salt, solvate, or solvate of a salt thereof.

2. The compound of the formula (I) as claimed in claim 1 in which
A represents a group of the formula 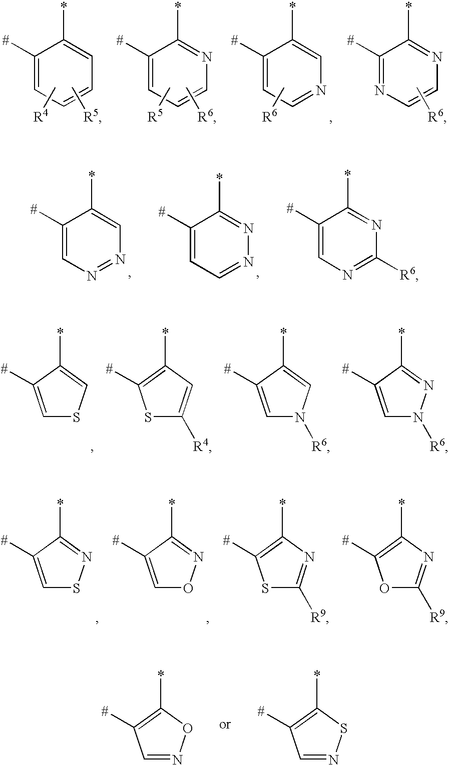
in which
R⁴ represents hydrogen, fluorine, chlorine, cyano, (C₁-C₆)-alkyl, trifluoromethyl, (C₃-C₇)-cycloalkyl, aminocarbonyl, mono- or di-(C₁-C₆)-alkylaminocarbonyl,
R⁵ represents hydrogen, fluorine, chlorine, cyano, (C₁-C₆)-alkyl, (C₃-C₇)-cycloalkyl, (C₁-C₆)-alkoxy, trifluoromethoxy, hydroxyl, amino, mono- or di-(C₁-C₆)-alkylamino, (C₃-C₇)-cycloalkylamino, (C₁-C₆)-alkanoylamino, (C₁-C₆)-alkoxycarbonylamino, hydroxycarbonyl or aminocarbonyl,
where (C₁-C₆)-alkyl, (C₁-C₆)-alkoxy, mono- and di-(C₁-C₆)-alkylamino for their part may each be substituted by hydroxyl, (C₁-C₄)-alkoxy, amino, mono- or di-(C₁-C₄)-alkylamino or (C₃-C₅)-cycloalkylamino,
R⁶ represents hydrogen, (C₁-C₆)-alkyl or (C₃-C₇)-cycloalkyl,
R⁹ represents hydrogen, (C₁-C₆)-alkyl, (C₃-C₇)-cycloalkyl, (C₁-C₆)-alkylthio or (C₁-C₆)-alkylsulfonyl
and
# and * represent the points of attachment to the —CO—NH-phenyl- and the —NH—CO-Z grouping.

3. The compound of the formula (I) as claimed in claim 1 in which
Z represents a group of the formula 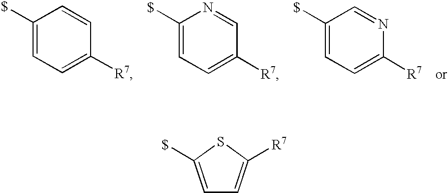
in which
R⁷ represents fluorine, chlorine, methyl or ethynyl
and
$ represents the point of attachment to the carbonyl group.

4. The compound of the formula (I) as claimed in claim 1 in which
n represents the number 1 or 2,
R¹ represents hydrogen,
R² represents hydrogen,
represents hydrogen, fluorine or methyl,
A represents a group of the formula 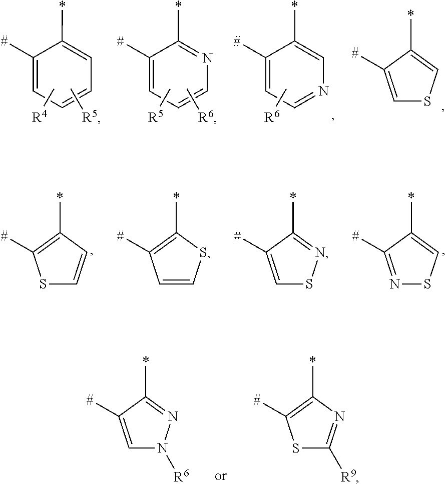
in which
R⁴ represents hydrogen, fluorine, chlorine, cyano, (C₁-C₄)-alkyl, trifluoromethyl, aminocarbonyl or di-(C₁-C₄)-alkylaminocarbonyl,
R⁵ represents hydrogen, fluorine, cyano, (C₁-C₄)-alkyl, (C₁-C₄)-alkoxy or mono- or di-(C₁-C₄)-alkylamino,
R⁶ represents hydrogen, (C₁-C₄)-alkyl or (C₃-C₅)-cycloalkyl,
R⁹ represents hydrogen, (C₁-C₄)-alkyl, (C₃-C₅)-cycloalkyl, (C₁-C₄)-alkylthio or (C₁-C₄)-alkylsulfonyl,
# represents the point of attachment to the —CO—NH-phenyl grouping
and
* represents the point of attachment to the —NH—CO-Z grouping,
and
Z represents a group of the formula 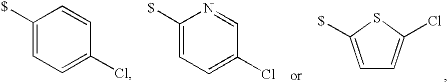
in which $ represents the point of attachment to the carbonyl group, or a salt, solvate, or solvate of a salt thereof.

5. A process for preparing compounds of the formula (I), as defined in claim 1, in which R¹ represents hydrogen, characterized in that a compound of the formula (II) 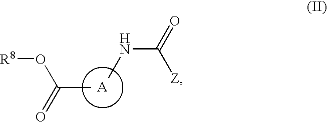
in which A and Z have the meanings given in claim 1 and
R⁸ represents hydrogen, methyl or ethyl,
is initially converted, with activation of the ester or the carboxylic acid function with a compound of the formula (III) 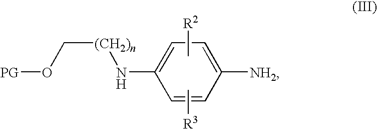
in which n, R² and R³ have the meanings given in claim 1
and
PG represents a hydroxyl protective group,
into a compound of the formula (IV) 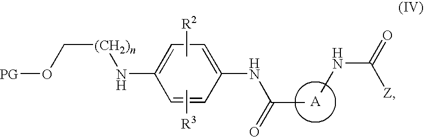
in which n, A, PG, Z, R² and R³ have the meanings given in claim 1, then either
[A] by removal of the protective group PG converted into a compound of the formula (V) 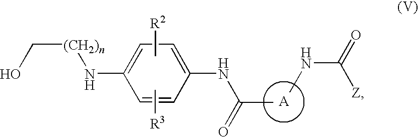
in which n, A, Z, R² and R³ have the meanings given in claim 1,
and the compound of the formula (V) is then in the presence of an acid converted with cyanogen bromide into a compound of the formula (I-A) 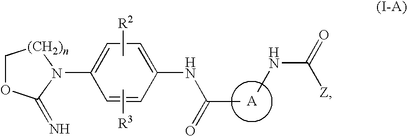
in which n, A, Z, R² and R³ have the meanings given in claim 1, or
[B] initially converted with cyanogen bromide into a compound of the formula (VI) 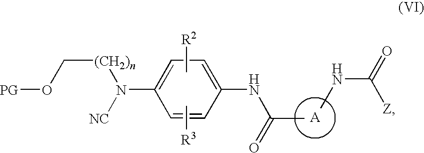
in which n, A, PG, Z, R² and R³ have the meanings given in claim 1,
then by removal of the protective group PG converted into a compound of the formula (VII) 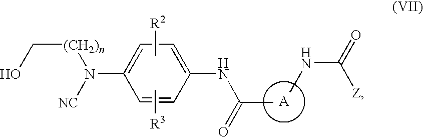
in which n, A, Z, R² and R³ have the meanings given in claim 1,
and the compound of the formula (VU) is then cyclized in the presence of an acid to a compound of the formula (I-A),
and the compound of the formula (I-A) is, if appropriate, converted with the appropriate (i) solvents and/or (ii) bases or acids into its solvate, salt, or solvate of a salt.

6. (canceled)

7. (canceled)

8. (canceled)

9. A pharmaceutical composition, comprising a compound of the formula (I) as defined in claim 1 in combination with an inert nontoxic, pharmaceutically suitable auxiliary.

10. A pharmaceutical composition, comprising a compound of the formula (I) as defined in claim 1 in combination with a further active compound.

11. (canceled)

12. A method for the treatment and/or prophylaxis of thromboembolic disorders in humans and animals, which comprises administering an anticoagulatory effective amount of at least one compound of the formula (I) as defined in claim 1 or a pharmaceutical composition as defined in claim 9 or 10.

13. A method for preventing blood coagulation in vitro, characterized in that an anticoagulatory effective amount of a compound of the formula (I) as defined in claim 1 is added.
